(19) United States
(12) Patent Application Publication
(10) Pub. No.: US 2009/0029855 A1
(43) Pub. Date: Jan. 29, 2009

(54) Quinoline Derivatives and Their Use as Pesticides

(76) Inventors: Michael Puhl, Lampertheim (DE); Wassilios Grammenos, Ludwigshafen (DE); Joachim Rheinheimer, Ludwigshafen (DE); Jan Klaas Lohmann, Ludwigshafen (DE); Michael Rack, Eppelheim (DE); Liliana Parra Rapado, Offenburg (DE); Christopher Koradin, Ludwigshafen (DE); Jurgen Langewald, Mannheim (DE); Deborah L. Culbertson, Fuquay Varina, NC (US); Douglas D. Anspaugh, Apex, NC (US); Hassan Oloumi-Sadeghi, Raleigh, NC (US); Henry Van Tuyl Cotter, Raleigh, NC (US); David G. Kuhn, Apex, NC (US)

Correspondence Address: BRINKS, HOFER, GILSON & LIONE, P.O. BOX 1340, MORRISVILLE, NC, 27560, US

(21) Appl. No.: 12/282,608

(22) PCT Filed: Mar. 12, 2007
(86) PCT No.: PCT/EP2007/052262

§ 371 Date: Sep. 11, 2008

Related U.S. Application Data
(60) Provisional application No. 60/782,429, filed on Mar. 15, 2006.

(51) Int. Cl. A01N 25/26; C07D 215/14; A01N 43/42; A01N 59/26; A01P 5/00; A01P 7/04

(52) U.S. Cl. 504/100; 546/172; 514/311; 424/601

(57) ABSTRACT

This invention relates to quinoline derivatives of formula I

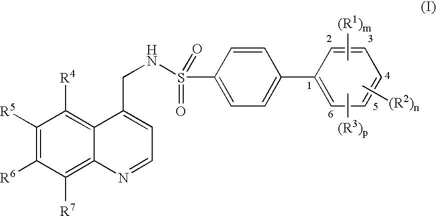

wherein R¹, R², R³ are each independently halogen, hydroxy, cyano, amino, nitro, C₁-C₆-alkyl, C₂-C₆-alkenyl, C₂-C₆-alkynyl, C₃-C₇-cycloalkyl, C₃-C₇-cycloalkyl-C₁-C₄-alkyl, C₁-C₆-alkoxy, C₂-C₆-alkenyloxy, C₂-C₆-alkynyloxy, C₁-C₄-alkoxy-C₁-C₄-alkoxy, C₃-C₇-cycloalkyl-C₁-C₄-alkoxy, C(OH)(CF₃)₂, C₁-C₆-haloalkyl, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C(R^{a})═O, C(R^{a})═NOR^{b}, C(═O)OR^{x}, or C(═O)NR^{x}R^{y};
R^{a} is hydrogen or C₁-C₄-alkyl;
R^{b} is hydrogen, C₁-C₄-alkyl, C₂-C₄-alkenyl, C₂-C₄-alkynyl, C₁-C₄-haloalkyl, or C₂-C₄-haloalkenyl;
R^{x}, R^{y} are each independently hydrogen, C₁-C₄-alkyl, C₁-C₄-haloalkyl, C₁-C₄-alkoxy-C₁-C₄-alkyl, C₁-C₄-thioalkyl-C₁-C₄-alkyl, C₁-C₄-alkyl-S(═O)C₁-C₄-alkyl, C₁-C₄-alkyl-S(═O)₂C₁-C₄-alkyl, C₃-C₆-cyloalkyl, C₁-C₄-alkyl-C₃-C₆-cycloalkyl, C₃-C₆-alkenyl, C₃-C₆-alkinyl;
R⁴, R⁵, R⁶, R⁷ are each independently hydrogen, halogen, cyano, amino, nitro, hydroxy, C₁-C₆-alkyl, C₁-C₆-alkoxy, C₁-C₆-haloalkyl, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, or C(═O)OR^{c};
R^{c} is hydrogen, C₁-C₆-alkyl, C₂-C₆-alkenyl, or C₂-C₆-alkinyl;
m and n are each independently 1, 2, 3, 4, or 5;
p is 0, 1, 2, 3, 4, or 5;
   and the N-oxides, enantiomers, diastereomers and salts thereof, processes and intermediates for the preparation of compounds I, use of compounds I for combating insects, acarids, or nematodes and a method for treating, controlling, preventing or protecting animals against infestation or infection by parasites using compounds I.

The present invention relates to quinoline derivatives of formula I

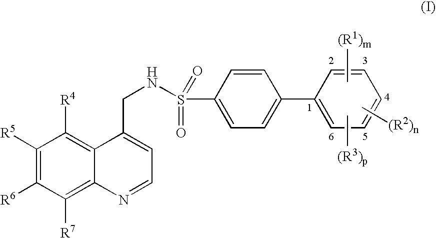

R¹, R², R³ are each independently halogen, hydroxy, cyano, amino, nitro, C₁-C₆-alkyl, C₂-C₆-alkenyl, C₂-C₆-alkynyl, C₃-C₇-cycloalkyl, C₃-C₇-cycloalkyl-C₁-C₄-alkyl, C₁-C₆-alkoxy, C₂-C₆-alkenyloxy, C₂-C₆-alkynyloxy, C₁-C₄-alkoxy-C₁-C₄-alkoxy, C₃-C₇-cycloalkyl-C₁-C₄-alkoxy, C(OH)(CF₃)₂, C₁-C₆-haloalkyl, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C(R^{a})═O, C(R^{a})═NOR^{b}, C(═O)OR^{x}, or C(═O)NR^{x}R^{y};
   R^{a} is hydrogen or C₁-C₄-alkyl;
   R^{b} is hydrogen, C₁-C₄-alkyl, C₂-C₄-alkenyl, C₂-C₄-alkynyl, C₁-C₄-haloalkyl, or C₂-C₄-haloalkenyl;
   R^{x}, R^{y} are each independently hydrogen, C₁-C₄-alkyl, C₁-C₄-haloalkyl, C₁-C₄-alkoxy-C₁-C₄-alkyl, C₁-C₄-thioalkyl-C₁-C₄-alkyl, C₁-C₄-alkyl-S(═O)C₁-C₄-alkyl, C₁-C₄-alkyl-S(═O)₂C₁-C₄-alkyl, C₃-C₆-cyloalkyl, C₁-C₄-alkyl-C₃-C₆-cycloalkyl, C₃-C₆-alkenyl, C₃-C₆-alkinyl;
R⁴, R⁵, R⁶, R⁷ are each independently hydrogen, halogen, cyano, amino, nitro, hydroxy, C₁-C₆-alkyl, C₁-C₆-alkoxy, C₁-C₆-haloalkyl, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, or C(═O)OR^{c};
   R^{c} is hydrogen, C₁-C₆-alkyl, C₂-C₆-alkenyl, or C₂-C₆-alkinyl;
m and n are each independently 1, 2, 3, 4, or 5;
p is 0, 1, 2, 3, 4, or 5;
   and the N-oxides, enantiomers, diastereomers and salts thereof.

In addition, the present invention relates to processes for preparing the compounds I, pesticidal compositions comprising compounds I and methods for the control of insects, acarids, or nematodes by contacting the insect, acarid, or nematode or their food supply, habitat or breeding grounds with a pesticidally effective amount of compounds or compositions of formula I.

Moreover, the present invention also relates to a method of protecting growing plants from attack or infestation by insects, acarids, or nematodes by applying to the plants, or to the soil or water in which they are growing, a pesticidally effective amount of compositions or compounds of formula I.

This invention also provides a method for treating, controlling, preventing or protecting animals against infestation or infection by parasites which comprises orally, topically or parenterally administering or applying to the animals a parasiticidally effective amount of compositions or compounds of formula I.

In spite of the commercial insecticides and acaricides available today, damage to crops, both growing and harvested, caused by pests still occurs. Therefore, there is continuing need to develop new and more effective pesticidal agents.

It was therefore an object of the present invention to provide new pesticidal compositions, new compounds and new methods for the control of insects, acarids, or nematodes and of protecting growing plants from attack or infestation by insects, acarids, and nematodes.

We have found that these objects are achieved by the compositions and the compounds of formula I. Furthermore, we have found processes for preparing the compounds of formula I.

Fungicidal quinoline methyleneamino sulfonamides have been described in WO 05/33081. No specific mention is made of quinoline methyleneamino sulfonamides carrying a biphenyl moiety at the sulfonamide group. Co-pending application U.S. 60/662,411 inter alia describes specific pesticidal quinoline methyleneamino sulfonamides carrying a biphenyl moiety at the sulfonamide group wherein the biphenyl moiety may be substituted in position 4 (as numbered above). Compounds wherein the biphenyl group carries more than 1 substituent are not specifically disclosed.

Generally, quinoline derivatives of formula (I) can be obtained by reaction of intermediates (II) with boronic acids (III) by a Suzuki coupling, wherein the variables in these compounds have the meaning as defined above for quinoline derivatives of formula (I) and L is a leaving group:

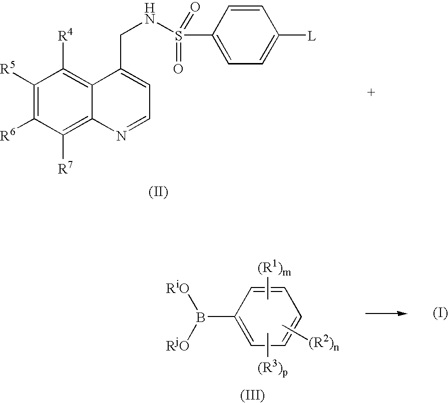

The reaction is usually carried out at temperatures of from 20° C. to 180° C., preferably from 40° C. to 120° C., in an inert organic solvent in the presence of a base and a catalyst, in particular a palladium catalyst, such as for example described in the following literature: Synth. Commun. Vol. 11, p. 513 (1981); Acc. Chem. Res. Vol. 15, pp. 178-184 (1982); Chem. Rev. Vol. 95, pp. 2457-2483 (1995); Organic Letters Vol. 6 (16), p. 2808 (2004); “Metal catalyzed cross coupling reactions”, 2^{nd} Edition, Wiley, VCH 2005 (Eds. De Meijere, Diederich); “Handbook of organopalladium chemistry for organic synthesis” (Eds Negishi), Wiley, Interscience, New York, 2002; “Handbook of functionalized organometallics”, (Ed. P. Knochel), Wiley, VCH, 2005.

Suitable leaving groups L in compounds (II) are halogen, preferably chloro, bromo or iodo, alkylcarbonylate, benzoate, alkylsulfonate, haloalkylsulfonate or arylsulfonate, most preferably chloro.

Suitable boronic acids are those wherein the variables Rⁱ and R^{j} have the meaning hydrogen or C₁-C₄-alkyl, or Rⁱ and R^{j} together form ethylene or propylene bridge the carbon atoms of which may all or in part be substituted by methyl groups.

Suitable catalysts are in tetrakis(triphenylphosphine)palladium(0); bis(triphenylphosphine)palladium(II) chloride; bis(acetonitrile)palladium(II) chloride; [1,1′-bis(diphenylphosphino)ferrocene]-palladium(II) chloride complex; bis[bis-(1,2-diphenylphosphino)ethane]palladium(0); bis(bis-(1,2-diphenylphosphino)butane]-palladium(II) chloride; palladium(II) acetate; palladium(II) chloride; and palladium(II) acetate/tri-o-tolylphosphine complex or mixtures of phosphines and Pd salts or phosphines and Pd-complexes e.g. dibenzylideneacetone-palladium and tritertbutyl-phosphine (or its tetrafluoroborate), tris cyclohexylphosphine; or a polymer-bound Pd-triphenylphosphine catalyst system.

Suitable solvents are aliphatic hydrocarbons, such as pentane, hexane, cyclohexane and petroleum ether, aromatic hydrocarbons, such as toluene, o-, m- and p-xylene, ethers, such as diisopropyl ether, tert.-butyl methyl ether, dioxane, anisole and tetrahydrofuran and dimethoxyethane, ketones, such as acetone, methyl ethyl ketone, diethyl ketone and tert.-butyl methyl ketone, and also acetonitrile, dimethyl sulfoxide, dimethyl-formamide and dimethylacetamide, particularly preferably ethers, such as tetrahydrofuran, dioxane and dimethoxyethane. It is also possible to use mixtures of the solvents mentioned, or mixtures with water.

Suitable bases are, in general, inorganic compounds, such as alkali metal and alkaline earth metal oxides, such as lithium oxide, sodium oxide, calcium oxide and magnesium oxide, alkali metal and alkaline earth metal carbonates, such as lithium carbonate, sodium carbonate, potassium carbonate, caesium carbonate and calcium carbonate, and also alkali metal bicarbonates, such as sodium bicarbonate, alkali metal and alkaline earth metal alkoxides, such as sodium methoxide, sodium ethoxide, potassium ethoxide and potassium tert.-butoxide, moreover organic bases, for example tertiary amines, such as trimethylamine, triethylamine, triisopropylethylamine and N-methylpiperidine, pyridine, substituted pyridines, such as collidine, lutidine and 4-dimethylaminopyridine, and also bicyclic amines. Particular preference is given to bases such as sodium carbonate, potassium carbonate, caesium carbonate, triethylamine and sodium-bicarbonate.

The base is used in a 1:1 to 1:10, preferably a 1:1.5 to 5 molar ratio relative to 1 mole of compounds (II), the boronic acid is used in a 1:1 to 1:5 ratio, preferably a 1:1 to 1:2.5 molar ratio relative to 1 mole of compounds (II). In some cases it may be beneficial for easy purification to use the boronic acid in a substoechiometric amount of from 0.7:1 to 0.99:1, relative to 1 mole of compounds (II).

After completion of the reaction, the compounds of formula (I) can be isolated by employing conventional methods such as adding the reaction mixture to water, extracting with an organic solvent, concentrating the extract and the like. The isolated compounds (I) can be purified by a technique such as chromatography, recrystallization and the like, if necessary.

It is also possible to add a scavenger to the reaction mixtures to remove byproducts or unreacted starting materials by binding to those and simple filtration. For details see “Synthesis and purification catalog”, Argonaut, 2003 and literature cited therein.

Boronic acids or esters (III) are commercially available or can be prepared according to “Science of Synthesis” Vol. 6, Thieme, 2005; WO 02/042275; Synlett 2003, (8) p. 1204; J. Org. Chem., 2003, 68, p. 3729, Synthesis, 2000, p. 442, J. Org. Chem., 1995, 60, p. 750; or “Handbook of functionalized organometallics”, (Ed. P. Knochel), Wiley, VCH, 2005.

Intermediates (II) can be obtained by reaction of sulfonylchlorides (IV) with quinolines (V), wherein the variables in these compounds have the meaning as defined above for quinoline derivatives of formula (I), and L and L¹ are leaving groups, in the presence of a base:

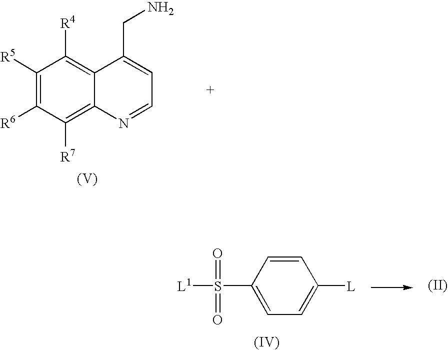

This reaction is usually carried out at temperatures of from −30° C. to 120° C., preferably from −10° C. to 100° C., in an inert organic solvent in the presence of a base (lit. eg: Lieb. Ann. Chem. P. 641, 1990.

Suitable solvents are aliphatic hydrocarbons, such as pentane, hexane, cyclohexane and petroleum ether, aromatic hydrocarbons, such as toluene, o-, m- and p-xylene, halogenated hydrocarbons, such as methylene chloride, chloroform and chlorobenzene, ethers, such as diethyl ether, diisopropyl ether, tert.-butyl methyl ether, dioxane, anisole and tetrahydrofuran, nitriles, such as acetonitrile and propionitrile, ketones, such as acetone, methyl ethyl ketone, diethyl ketone and tert.-butyl methyl ketone, and also dimethyl sulfoxide, dimethylformamide and dimethylacetamide, preferably tetrahydrofuran, methyltertbutylether, methylenechloride, chloroform, acetonitrile, toluene or dimethylformamide. It is also possible to use mixtures of the solvents mentioned.

Suitable bases are, in general, inorganic compounds, such as alkali metal and alkaline earth metal hydroxides, such as lithium hydroxide, sodium hydroxide, potassium hydroxide and calcium hydroxide, alkali metal and alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal and alkaline earth metal carbonates, such as lithium carbonate, potassium carbonate and calcium carbonate, and also alkali metal bicarbonates, such as sodium bicarbonate, moreover organic bases, for example tertiary amines, such as trimethylamine, triethylamine, triisopropylethylamine and N-methylpiperidine, pyridine, substituted pyridines, such as collidine, lutidine and 4-dimethylaminopyridine, and also bicyclic amines. Particular preference is given to pyridine, triethylamine and potassium carbonate. The bases are generally employed in equimolar amounts, in excess or, if appropriate, as solvent. The excess of base is typically 0.5 to 5 molar equivalents relative to 1 mole of compounds (V).

The starting materials are generally reacted with one another in equimolar amounts.

If sulfonylchlorides (IV) are not commercially available, they can be obtained according to procedures known in the art.

Quinolines (V) are known from the literature or are commercially available (e.g.: 4-methylenaminoquinoline: CAS-Nr 5632-13-3; 6-chloro-4-methylenaminoquinoline: CAS-Nr 859814-05-5; 6-methoxy-4-methylenaminoquinoline: CAS-Nr 708261-71-6; 8-hydroxy-4-methylenaminoquinoline: CAS-Nr 33976-91-9; 6-methoxy-8-chloro-4-methylenaminoquinoline: CAS-Nr 857207-07-9), or they can be prepared from quinoline precursors, (VI) wherein X is as defined in the following scheme by reduction:

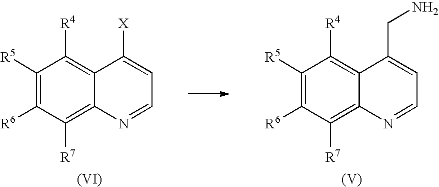

VIa: X═CH₂NHOH VId: X═CHO
VIb: X═CN VIe: X═CH₃
VIc: X═C(═O)NH₂ VIf: X═Cl, Br

Methods of this reduction can be found in the literature e.g. in Houben-Weyl, Band 10/4, Thieme, Stuttgart, 1968; Band 11/2, 1957; Band E5, 1985; J. Heterocycl. Chem., 1997, 34 (6), pp. 1661-1667; J. Chem. Soc. 1954, p. 1165; Heterocycles, 41(4), pp. 675-688, 1995; J. Org. Chem., 1982, 47, p. 3153; Heterocycles, 1996, 43 (9), pp. 1893-1900; J. Prakt. Chem-Chem. Ztg. 336(8), pp. 695-697, 1994; or are known to those skilled in the art.

The oxims (VIa) can be prepared from either the respective aldehyd (X═CHO; compounds (VId)) or the methylderivative (X═CH₃; compounds (VIe)), as described in Houben-Weyl, Band 10/4, Thieme, Stuttgart, 1968; Band 11/2, 1957; Band E5, 1985; J. Prakt. Chem-Chem. Ztg. 336(8), pp. 695-697, 1994; Tetrahedron Lett. 42(39), pp. 6815-6818, 2001; or Heterocycles, 29(9), pp. 1741-1760, 1989.

The aldehyds (VId) are commercially available (e.g 6-chloroquinoline-4 aldehyd, 7-methoxy quinolin-4-aldehyde, quinoline-4-aldehyd) or can be synthesized from a 4-methyl quinoline as outlined in J. Org. Chem. 51(4), pp. 536-537, 1986, or from a haloderivative (X=halogen, compounds (VIf)) as shown in Eur. J. Org. Chem., 2003, (8), pp. 1576-1588; Tetrahedron Lett. 1999, 40 (19), pp. 3719-3722; Tetrahedron, 1999, 55 (41), pp. 12149-12156.

The methyl derivatives (VIe) are commercially available (e.g. 6-chloro 4-methylquinoline; 6,8-dimethoxy-quinoline) or can be synthesized according to “Science of Synthesis”, Vol 15, Thieme, Stuttgart, 2005.

The nitrils (VIb) can be prepared either from the respective halogen derivative (VIf) (X=halogen, preferably chlorine, bromine or iodine,) by reaction with a cyanide source with or without additional catalysts, as described e.g. in Tetrahedron Lett. 42(38), pp. 6707-6710, 2001; Chem. Eur. J., 2003, 9 (8), pp. 1828-1836; Chem. Commun. (Cambridge), 2004, (12), pp. 1388-1389; J. Organomet. Chem. 2004, 689 (24), pp. 4576-4583; or J. Chem. Soc. Perk. T., 1 (16), pp. 2323-2326, 1999. Alternatively, the amide or oxime may be dehydrated to the corresponding nitrile (VIb) as outlined in “Synthesis”, Stuttgart, (10), pp. 943-944, 1992; or literature cited therein; or Heterocycl. Chem. 1997, 34 (6), pp. 1661-1667.

The 4-halogene quinolines (VIf) are either commercially available or can be synthesized according to “Science of Synthesis”, Vol 15, Thieme, Stuttgart, 2005 or e.g. according to the following literature or citations therein: 4-chloro-6,7-dimethoxy quinoline: Journal Med. Chem. 48(5), p. 1359, 2005; 4-chloro-5,7-dichloro quinoline: Indian, 187817, 29 Jun. 2002; 4-chloro-7-chloro quinoline: Tetrahedron, 60 (13), p. 3017, 2004; 4-chloro-7-trifluoromethyl quinoline: Tetrahedron lett., 31(8), p. 1093, 1990; 4-chloro-7,8-dimethoxy quinoline: Tetrahedron, 41 (15), p. 3033, 1985; 4-chloro-8-methoxy quinoline: Chem. Berichte 118(4), p. 1556, 1985; 4-chloro-(6 or 7 or 8)-iodo quinoline, 4-bromo-(6 or 7 or 8)-iodo quinoline, 4-iodo-(6 or 7 or 8)-iodo quinoline: J. Med. Chem., 21(3), p. 268, 1978.

Further methods to build up appropriate precursors or modify substitution pattern can be found in “Synthesis”, Stuttgart (1), pp. 31-32, 1993; Tetrahedron, 1993, 49 (24), pp. 5315-5326; “Methods in Science of Synthesis”, Band 15, and literature cited therein; Bioorg. Med. Chem. Lett. 1997, 7 (23), pp. 2935-2940; J. Am. Chem. Soc., 1946, 68, p. 1264; or Org. Synth. 1955, III, p. 272.

In same cases it can be beneficial in terms of ease of work up or purification to perform the reduction of compounds (VI) to compounds (V) and the reaction of the amine (V) with the compound (IV) in one pot without isolating compounds (V).

Some of the intermediates of formula II are novel and exhibit pesticidal activity. These are also subject of this invention.

Especially, the intermediates of formula II.1 are subject of the present invention:

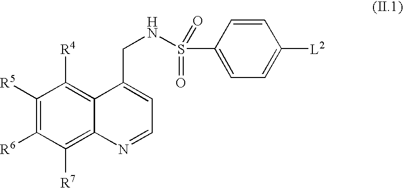

wherein L² is chlorine, bromine or iodine and the other variables are as defined above for formula I, with the exemption of compounds wherein R⁴ and R⁷ are hydrogen and R⁵ and R⁶ are the same and selected from the group consisting of hydrogen, methyl, fluorine, chlorine, methoxy and trifluoromethoxy.

If individual compounds I are not obtainable by the routes described above, they can be prepared by derivatization of other compounds I or by customary modifications of the synthesis routes described.

The preparation of the compounds of formula I may lead to them being obtained as isomer mixtures (stereoisomers, enantiomers). If desired, these can be resolved by the methods customary for this purpose, such as crystallization or chromatography, also on optically active adsorbate, to give the pure isomers.

The inventive compounds I may be present in different crystalline modifications which may differ in their biological activity. These are also subject of the present invention.

Agronomically acceptable salts of the compounds I can be formed in a customary manner, e.g. by reaction with an acid of the anion in question.

In this specification and in the claims, reference will be made to a number of terms that shall be defined to have the following meanings:

“Salt” as used herein includes adducts of compounds I with maleic acid, dimaleic acid, fumaric acid, difumaric acid, methane sulfenic acid, methane sulfonic acid, and succinic acid. Moreover, included as “salts” are those that can form with, for example, amines, metals, alkaline earth metal bases or quaternary ammonium bases, including zwitterions. Suitable metal and alkaline earth metal hydroxides as salt formers include the salts of barium, aluminum, nickel, copper, manganese, cobalt zinc, iron, silver, lithium, sodium, potassium, magnesium or calcium. Additional salt formers include chloride, sulfate, acetate, carbonate, hydride, and hydroxide. Desirable salts include adducts of compounds I with maleic acid, dimaleic acid, fumaric acid, difumaric acid, and methane sulfonic acid.

“Halogen” will be taken to mean fluoro, chloro, bromo and iodo.

The term “alkyl” as used herein refers to a branched or unbranched saturated hydro-carbon group having 1 to 6 carbon atoms, for example methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl.

The term “haloalkyl” as used herein refers to a straight-chain or branched alkyl group having 1 to 6 carbon atoms (as mentioned above), where some or all of the hydrogen atoms in these groups may be replaced by halogen atoms as mentioned above, for example C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl and pentafluoroethyl;

Similarly, “alkoxy” and “alkylthio” refer to straight-chain or branched alkyl groups having 1 to 6 carbon atoms (as mentioned above) bonded through oxygen or sulfur linkages, respectively, at any bond in the alkyl group. Examples include methoxy, ethoxy, propoxy, isopropoxy, methylthio, ethylthio, propylthio, isopropylthio, and n-butylthio.

Similarly, “alkylsulfinyl” and “alkylsulfonyl” refer to straight-chain or branched alkyl groups having 1 to 6 carbon atoms (as mentioned above) bonded through —S(═O)— or —S(═O)₂-linkages, respectively, at any bond in the alkyl group. Examples include methyl-sulfinyl and methylsulfonyl.

The term “alkenyl” as used herein intends a branched or unbranched unsaturated hydrocarbon group having 2 to 6 carbon atoms and a double bond in any position, such as ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl; 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;

The term “alkynyl” as used herein refers to a branched or unbranched unsaturated hydrocarbon group having 2 to 6 carbon atoms and containing at least one triple bond, such as ethynyl, propynyl, 1-butynyl, 2-butynyl, and the like.

Similarly, “alkenyloxy” and “alkynyloxy” refer to straight-chain or branched alkyl groups having 2 to 6 carbon atoms (as mentioned above) bonded through an oxygen linkage, at any bond in the alkenyl group, or at any carbon atom of the alkynyl group which is not vicinal to the carbon atom of the triple bond, e.g. allyloxy or propargyloxy.

The term “cycloalkyl” as used herein refers to monocyclic 3- to 7-membered saturated carbon atom rings, such as cyclopropyl, cyclobutyl, cyclopentyl, cyclohexyl, or cycloheptyl.

With respect to the intended use of the compounds of formula I, particular preference is given to the following meanings of the substituents, in each case on their own or in combination:

Quinoline derivatives of formula I according to claim 1, wherein

R¹, R², R³ are each independently halogen, cyano, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-haloalkyl, C₁-C₆-haloalkoxy, C₁-C₆-alkylthio, C₁-C₆-haloalkylthio, C₁-C₆-alkylsulfonyl, C₁-C₆-haloalkylsulfonyl, or C(R^{a})═NOR^{b}, more preferably fluorine, chlorine, bromine, cyano, C₁-C₆-alkyl, C₁-C₆-alkoxy, C₁-C₆-haloalkyl, or C₁-C₆-haloalkoxy;
m and n are each independently 1, 2, 3, 4, or 5; and
p is 0, 1, 2, 3, 4, or 5.

Quinoline derivatives of formula I according to claim 1, wherein R¹, R², R³ are each independently fluorine, chlorine, bromine, cyano, C₁-C₄-alkyl, C₁-C₆-cycloalkyl, C₁-C₄-alkoxy, C₂-C₃-alkenyloxy, C₂-C₃-alkynyloxy, C₁-C₄-haloalkyl, C₁-C₄-haloalkoxy, C₁-C₄-alkylthio, C₁-C₄-haloalkylthio, C₁-C₄-alkylsulfonyl, C₁-C₂-haloalkylsulfonyl; and the sum of m, n and p equals 2 or 3.

Quinoline derivatives of formula I according to claim 1, wherein R⁴, R⁵, R⁶, R⁷ are each independently hydrogen, halogen, C₁-C₆-alkyl, C₁-C₆-alkoxy, C₁-C₆-haloalkyl, C₁-C₆-haloalkoxy, or C₁-C₆-alkylthio, preferably fluorine, chlorine, bromine, iodine, C₁-C₄-alkyl, C₁-C₄-alkoxy, C₁-C₄-haloalkyl, C₁-C₄-haloalkoxy, or C₁-C₄-alkylthio.

A compound of formula I wherein the sum of m, n and p equals 2 or 3.

A compound of formula I wherein the position marked “2” in the biphenylmoiety is substituted with fluoro, chloro, bromo, methyl, methoxy, trifluoromethyl, methylthio, trifluoromethylthio, difluormethoxy or trifluormethoxy.

A compound of formula I wherein the position marked “2” in the biphenylmoiety is substituted with fluoro, chloro, bromo, methyl, methoxy, trifluoromethyl, methylthio, trifluoromethylthio, difluormethoxy or trifluormethoxy, and the sum of m, n and p equals 2 or 3.

A compound of formula I wherein the position marked “3” in the biphenylmoiety is substituted with fluoro, chloro, bromo, methyl, methoxy, trifluoromethyl, methylthio, trifluoromethylthio, difluormethoxy or trifluormethoxy.

A compound of formula I wherein the position marked “3” in the biphenylmoiety is substituted with fluoro, chloro, bromo, methyl, methoxy, trifluoromethyl, methylthio, trifluoromethylthio, difluormethoxy or trifluormethoxy, and the sum of m, n and p equals 2 or 3.

A compound of formula I.1 wherein the biphenylmoiety has the substitution pattern as in the following scheme, and R¹ and R² are as defined hereinabove for compounds of formula I:

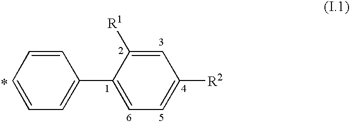

A compound of formula I.1 wherein the biphenylmoiety has the substitution pattern as depicted above, R¹ is selected from the group fluoro, chloro, bromo, methyl, methoxy, trifluoromethyl, methylthio, trifluoromethylthio, difluormethoxy and trifluormethoxy, and R² is as defined hereinabove for compounds of formula I.

A compound of formula I.1 as defined in the two preceding paragraphs which carries one further substituent selected from R³.

A compound of formula I.2 wherein the biphenylmoiety has the substitution pattern:

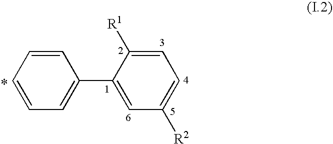

A compound of formula I.2 which carries one further substituent selected from R³.

A compound of formula I.3 wherein the biphenylmoiety has the substitution pattern:

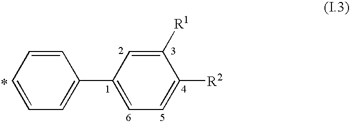

A compound of formula I.3 which carries one further substituent selected from R³.

A compound of formula I wherein the quinoline moiety is selected from table A:

TABLE A

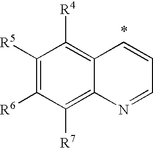

| Quinoline moiety | | | | |
|---|---|---|---|---|
| no. | R⁴ | R⁵ | R⁶ | R⁷ |
| Q-1 | H | H | H | H |
| Q-2 | F | H | H | H |
| Q-3 | H | F | H | H |
| Q-4 | H | H | F | H |
| Q-5 | H | H | H | F |
| Q-6 | Cl | H | H | H |
| Q-7 | H | Cl | H | H |
| Q-8 | H | H | Cl | H |
| Q-9 | H | H | H | Cl |
| Q-10 | CH₃ | H | H | H |
| Q-11 | H | CH₃ | H | H |
| Q-12 | H | H | CH₃ | H |
| Q-13 | H | H | H | CH₃ |
| Q-14 | OCH₃ | H | H | H |
| Q-15 | H | OCH₃ | H | H |
| Q-16 | H | H | OCH₃ | H |
| Q-17 | H | H | H | OCH₃ |
| Q-18 | CF₃ | H | H | H |
| Q-19 | H | CF₃ | H | H |
| Q-20 | H | H | CF₃ | H |
| Q-21 | H | H | H | CF₃ |
| Q-22 | OCF₃ | H | H | H |
| Q-23 | H | OCF₃ | H | H |
| Q-24 | H | H | OCF₃ | H |
| Q-25 | H | H | H | OCF₃ |
| Q-26 | Cl | Cl | H | H |
| Q-27 | Cl | H | Cl | H |
| Q-28 | Cl | H | H | Cl |
| Q-29 | H | Cl | Cl | H |
| Q-30 | H | Cl | H | Cl |
| Q-31 | H | H | Cl | Cl |
| Q-32 | F | F | H | H |
| Q-33 | F | H | F | H |
| Q-34 | F | H | H | F |
| Q-35 | H | F | F | H |
| Q-36 | H | F | H | F |
| Q-37 | H | H | F | F |
| Q-38 | CH₃ | CH₃ | H | H |
| Q-39 | CH₃ | H | CH₃ | H |
| Q-40 | CH₃ | H | H | CH₃ |
| Q-41 | H | CH₃ | CH₃ | H |
| Q-42 | H | CH₃ | H | CH3 |
| Q-43 | H | H | CH₃ | CH₃ |
| Q-44 | OCH₃ | OCH₃ | H | H |
| Q-45 | OCH₃ | H | OCH₃ | H |
| Q-46 | OCH₃ | H | H | OCH₃ |
| Q-47 | H | OCH₃ | OCH₃ | H |
| Q-48 | H | OCH₃ | H | OCH₃ |
| Q-49 | H | H | OCH₃ | OCH₃ |
| Q-50 | CF₃ | CF₃ | H | H |
| Q-51 | CF₃ | H | CF₃ | H |
| Q-52 | CF₃ | H | H | CF₃ |
| Q-53 | H | CF₃ | CF₃ | H |
| Q-54 | H | CF₃ | H | CF₃ |
| Q-55 | H | H | CF₃ | CF₃ |

*denotes the bonding site to the backbone

With respect to their use, particular preference is given to the compounds I compiled in the tables below. Moreover, the groups mentioned for a substituent in the tables are on their own, independently of the combination in which they are mentioned, a particularly preferred embodiment of the substituent in question.

### Table 1

Compounds of the formula IA wherein Q denotes Q-1, p is zero, and the combination of (R¹)ₘ and (R²)ₙ in each case corresponds to a combination no. of Table B.

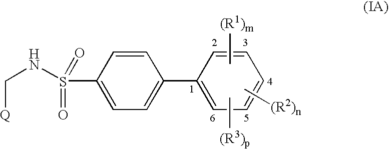

### Table 2

Compounds of the formula IA wherein Q denotes Q-2, p is zero, and the combination of (R¹)ₘ and (R²)ₙ in each case corresponds to a combination no. of Table B.

### Table 3

Compounds of the formula IA wherein Q denotes Q-3, p is zero, and the combination of (R¹)ₘ and (R²)ₙ in each case corresponds to a combination no. of Table B.

### Table 4

Compounds of the formula IA wherein Q denotes Q-4, p is zero, and the combination of (R¹)ₘ and (R²)ₙ in each case corresponds to a combination no. of Table B.

### Table 5

Compounds of the formula IA wherein Q denotes Q-5, p is zero, and the combination of (R¹)ₘ and (R²)ₙ in each case corresponds to a combination no. of Table B.

### Table 6

Compounds of the formula IA wherein Q denotes Q-6, p is zero, and the combination of (R¹)ₘ and (R²)ₙ in each case corresponds to a combination no. of Table B.

### Table 7

Compounds of the formula IA wherein Q denotes Q-8, p is zero, and the combination of (R¹)ₘ and (R²)ₙ in each case corresponds to a combination no. of Table B.

### Table 8

Compounds of the formula IA wherein Q denotes Q-10, p is zero, and the combination of (R¹)ₘ and (R²)ₙ in each case corresponds to a combination no. of Table B.

### Table 9

Compounds of the formula IA wherein Q denotes Q-12, p is zero, and the combination of (R¹)ₘ and (R²)ₙ in each case corresponds to a combination no. of Table B.

### Table 10

Compounds of the formula IA wherein Q denotes Q-14, p is zero, and the combination of (R¹)ₘ and (R²)ₙ in each case corresponds to a combination no. of Table B.

### Table 11

Compounds of the formula IA wherein Q denotes Q-16, p is zero, and the combination of (R¹)ₘ and (R²)ₙ in each case corresponds to a combination no. of Table B.

### Table 12

Compounds of the formula IA wherein Q denotes Q-18, p is zero, and the combination of (R¹)ₘ and (R²)ₙ in each case corresponds to a combination no. of Table B.

### Table 13

Compounds of the formula IA wherein Q denotes Q-20, p is zero, and the combination of (R¹)ₘ and (R²)ₙ in each case corresponds to a combination no. of Table B.

### Table 14

Compounds of the formula IA wherein Q denotes Q-22, p is zero, and the combination of (R¹)ₘ and (R²)ₙ in each case corresponds to a combination no. of Table B.

### Table 15

Compounds of the formula IA wherein Q denotes Q-24, p is zero, and the combination of (R¹)ₘ and (R²)ₙ in each case corresponds to a combination no. of Table B.

### Table 16

Compounds of the formula IA wherein Q denotes Q-27, p is zero, and the combination of (R¹)ₘ and (R²)ₙ in each case corresponds to a combination no. of Table B.

### Table 17

Compounds of the formula IA wherein Q denotes Q-33, p is zero, and the combination of (R¹)ₘ and (R²)ₙ in each case corresponds to a combination no. of Table B.

### Table 18

Compounds of the formula IA wherein Q denotes Q-39, p is zero, and the combination of (R¹)ₘ and (R²)ₙ in each case corresponds to a combination no. of Table B.

### Table 19

Compounds of the formula IA wherein Q denotes Q-45, p is zero, and the combination of (R¹)ₘ and (R²)ₙ in each case corresponds to a combination no. of Table B.

### Table 20

Compounds of the formula IA wherein Q denotes Q-51, p is zero, and the combination of (R¹)ₘ and (R²)ₙ in each case corresponds to a combination no. of Table B.

### Table 21

Compounds of the formula IA wherein Q denotes Q-1, (R³)ₚ is 4-F, and the combination of (R¹)ₘ and (R²)ₙ in each case corresponds to a combination no. of Table B selected from IA-469 to IA-1459.

Table 22

Compounds of the formula IA wherein Q denotes Q-1, (R³)ₚ is 4-Cl, and the combination of (R¹)ₘ and (R²)ₙ in each case corresponds to a combination no. of Table B selected from IA-469 to IA-1459.

### Table 23

Compounds of the formula IA wherein Q denotes Q-1, (R³)ₚ is 4-Br, and the combination of (R¹)ₘ and (R²)ₙ in each case corresponds to a combination no. of Table B selected from IA-469 to IA-1459.

### Table 24

Compounds of the formula IA wherein Q denotes Q-1, (R³)ₚ is 4-CH₃, and the combination of (R¹)ₘ and (R²)ₙ in each case corresponds to a combination no. of Table B selected from IA-469 to IA-1459.

### Table 25

Compounds of the formula IA wherein Q denotes Q-1, (R³)ₚ is 4-CH₂CH₃, and the combination of (R¹)ₘ and (R²)ₙ in each case corresponds to a combination no. of Table B selected from IA-469 to IA-1459.

### Table 26

Compounds of the formula IA wherein Q denotes Q-1, (R³)ₚ is 4-C(CH₃)₃, and the combination of (R¹)ₘ and (R²)ₙ in each case corresponds to a combination no. of Table B selected from IA-469 to IA-1459.

### Table 27

Compounds of the formula IA wherein Q denotes Q-1, (R³)ₚ is 4-OCH₃, and the combination of (R¹)ₘ and (R²)ₙ in each case corresponds to a combination no. of Table B selected from IA-469 to IA-14459.

### Table 28

Compounds of the formula IA wherein Q denotes Q-1, (R³)ₚ is 4-OCH₂CH₃, and the combination of (R¹)ₘ and (R²)ₙ in each case corresponds to a combination no. of Table B selected from IA-469 to IA-1459.

### Table 29

Compounds of the formula IA wherein Q denotes Q-1, (R³)ₚ is 4-CF₃, and the combination of (R¹)ₘ and (R²)ₙ in each case corresponds to a combination no. of Table B selected from IA-469 to IA-1459.

### Table 30

Compounds of the formula IA wherein Q denotes Q-1, (R³)ₚ is 4-CH(CF₃)₂, and the combination of (R¹)ₘ and (R²)ₙ in each case corresponds to a combination no. of Table B selected from IA-469 to IA-1459.

### Table 31

Compounds of the formula IA wherein Q denotes Q-1, (R³)ₚ is 4-CF(CF₃)₂, and the combination of (R¹)ₘ and (R²)ₙ in each case corresponds to a combination no. of Table B selected from IA-469 to IA-1459.

### Table 32

Compounds of the formula IA wherein Q denotes Q-1, (R³)ₚ is 4-C(OH)(CF₃)₂, and the combination of (R¹)ₘ and (R²)ₙ in each case corresponds to a combination no. of Table B selected from IA-469 to IA-1459.

### Table 33

Compounds of the formula IA wherein Q denotes Q-1, (R³)ₚ is 4-OCF₃, and the combination of (R¹)ₘ and (R²)ₙ in each case corresponds to a combination no. of Table B selected from IA-469 to IA-1459.

### Table 34

Compounds of the formula IA wherein Q denotes Q-1, (R³)ₚ is 4-OCHF₂, and the combination of (R¹)ₘ and (R²)ₙ in each case corresponds to a combination no. of Table B selected from IA-469 to IA-1459.

### Table 35

Compounds of the formula IA wherein Q denotes Q-1, (R³)ₚ is 4-SCF₃, and the combination of (R¹)ₘ and (R²)ₙ in each case corresponds to a combination no. of Table B selected from IA-469 to IA-1459.

### Table 36

Compounds of the formula IA wherein Q denotes Q-1, (R³)ₚ is 4-SO₂CF₃, and the combination of (R¹)ₘ and (R)ₙ in each case corresponds to a combination no. of Table B selected from IA-469 to IA-1459.

### Table 37

Compounds of the formula IA wherein Q denotes Q-1, (R³)ₚ is 4-CN, and the combination of (R¹)ₘ and (R²)ₙ in each case corresponds to a combination no. of Table B selected from IA-469 to IA-1459.

### Table 38

Compounds of the formula IA wherein Q denotes Q-1, (R³)ₚ is 4-SCH₃, and the combination of (R¹)ₘ and (R²)ₙ in each case corresponds to a combination no. of Table B selected from IA-469 to IA-1459.

### Table 39

Compounds of the formula IA wherein Q denotes Q-1, (R³)ₚ is 4-SO₂CH₃, and the combination of (R¹)ₘ and (R²)ₙ in each case corresponds to a combination no. of Table B selected from IA-469 to IA-1459.

### Table 40

Compounds of the formula IA wherein Q denotes Q-1, (R³)ₚ is 4-OCF₂CF₃, and the combination of (R¹)ₘ and (R²)ₙ in each case corresponds to a combination no. of Table B selected from IA-469 to IA-1459.

### Table 41

Compounds of the formula IA wherein Q denotes Q-1, (R³)ₚ is 4-OCH₂CHCH₂, and the combination of (R¹)ₘ and (R²)ₙ in each case corresponds to a combination no. of Table B selected from IA-469 to IA-1459.

### Table 42

Compounds of the formula IA wherein Q denotes Q-1, (R³)ₚ is 4-CH₂CCH, and the combination of (R¹)ₘ and (R²)ₙ in each case corresponds to a combination no. of Table B selected from IA-469 to IA-1459.

### Table 43

Compounds of the formula IA wherein Q denotes Q-1, (R³)ₚ is 4-CH₂CF₃, and the combination of (R¹)ₘ and (R²)ₙ in each case corresponds to a combination no. of Table B selected from IA-469 to IA-1459.

### Table 44

Compounds of the formula IA wherein Q denotes Q-1, (R³)ₚ is 4-CH₂CH₂OCH₃, and the combination of (R¹)ₘ and (R²)ₙ in each case corresponds to a combination no. of Table B selected from IA-469 to IA-1459.

### Table 45

Compounds of the formula IA wherein Q denotes Q-1, (R³)ₚ is 4-OC(CH₃)₃, and the combination of (R¹)ₘ and (R²)ₙ in each case corresponds to a combination no. of Table B selected from IA-469 to IA-1459.

### Table 46

Compounds of the formula IA wherein Q denotes Q-1, (R³)ₚ is 4-OCH₂-cyclo-C₃H₅, and the combination of (R¹)ₘ and (R²)ₙ in each case corresponds to a combination no. of Table B selected from IA-469 to IA-1459.

### Table 47

Compounds of the formula IA wherein Q denotes Q-1, (R³)ₚ is 5-F, and the combination of (R¹)ₘ and (R²)ₙ in each case corresponds to a combination no. of Table B selected from IA-1 to IA-702 and IA-1379 to IA-1459.

### Table 48

Compounds of the formula IA wherein Q denotes Q-1, (R³)ₚ is 5-Cl, and the combination of (R¹)ₘ and (R²)ₙ in each case corresponds to a combination no. of Table B selected from IA-1 to IA-702 and IA-1379 to IA-1459.

### Table 49

Compounds of the formula IA wherein Q denotes Q-1, (R³)ₚ is 5-Br, and the combination of (R¹)ₘ and (R²)ₙ in each case corresponds to a combination no. of Table B selected from IA-1 to IA-702 and IA-1379 to IA-1459.

### Table 50

Compounds of the formula IA wherein Q denotes Q-1, (R³)ₚ is 5-CH₃, and the combination of (R¹)ₘ and (R²)ₙ in each case corresponds to a combination no. of Table B selected from IA-1 to IA-702 and IA-1379 to IA-1459.

### Table 51

Compounds of the formula IA wherein Q denotes Q-1, (R³)ₚ is 5-OCH₃, and the combination of (R¹)ₘ and (R²)ₙ in each case corresponds to a combination no. of Table B selected from IA-1 to IA-702 and IA-1379 to IA-1459.

### Table 52

Compounds of the formula IA wherein Q denotes Q-1, (R³)ₚ is 5-CF₃, and the combination of (R¹)ₘ and (R²)ₙ in each case corresponds to a combination no. of Table B selected from IA-1 to IA-702 and IA-1379 to IA-1459.

### Table 53

Compounds of the formula IA wherein Q denotes Q-1, (R³)ₚ is 5-OCF₃, and the combination of (R¹)ₘ and (R²)ₙ in each case corresponds to a combination no. of Table B selected from IA-1 to IA-702 and IA-1379 to IA-1459.

### Table 54

Compounds of the formula IA wherein Q denotes Q-1, (R³)ₚ is 5-SCH₃, and the combination of (R¹)ₘ and (R²)ₙ in each case corresponds to a combination no. of Table B selected from IA-1 to IA-702 and IA-1379 to IA-1459.

### Table 55

Compounds of the formula IA wherein Q denotes Q-1, (R³)ₚ is 5-OCHF₂, and the combination of (R¹)ₘ and (R²)ₙ in each case corresponds to a combination no. of Table B selected from IA-1 to IA-702 and IA-1379 to IA-1459.

### Table 56

Compounds of the formula IA wherein Q denotes Q-5, (R³)ₚ is 4-F, and the combination of (R¹)ₘ and (R²)ₙ in each case corresponds to a combination no. of Table B selected from IA-469 to IA-1459.

### Table 57

Compounds of the formula IA wherein Q denotes Q-5, (R³)ₚ is 4-Cl, and the combination of (R¹)ₘ and (R²)ₙ in each case corresponds to a combination no. of Table B selected from IA-469 to IA-1459.

### Table 58

Compounds of the formula IA wherein Q denotes Q-5, (R³)ₚ is 4-Br, and the combination of (R¹)ₘ and (R²)ₙ in each case corresponds to a combination no. of Table B selected from IA-469 to IA-1459.

### Table 59

Compounds of the formula IA wherein Q denotes Q-5, (R³)ₚ is 4-CH₃, and the combination of (R¹)ₘ and (R²)ₙ in each case corresponds to a combination no. of Table B selected from IA-469 to IA-1459.

### Table 60

Compounds of the formula IA wherein Q denotes Q-5, (R³)ₚ is 4-CH₂CH₃, and the combination of (R¹)ₘ and (R²)ₙ in each case corresponds to a combination no. of Table B selected from IA-469 to IA-1459.

### Table 61

Compounds of the formula IA wherein Q denotes Q-5, (R³)ₚ is 4-C(CH₃)₃, and the combination of (R¹)ₘ and (R²)ₙ in each case corresponds to a combination no. of Table B selected from IA-469 to IA-1459.

### Table 62

Compounds of the formula IA wherein Q denotes Q-5, (R³)ₚ is 4-OCH₃, and the combination of (R¹)ₘ and (R²)ₙ in each case corresponds to a combination no. of Table B selected from IA-469 to IA-1459.

### Table 63

Compounds of the formula IA wherein Q denotes Q-5, (R³)ₚ is 4-OCH₂CH₃, and the combination of (R¹)ₘ and (R²)ₙ in each case corresponds to a combination no. of Table B selected from IA-469 to IA-1459.

### Table 64

Compounds of the formula IA wherein Q denotes Q-5, (R³)ₚ is 4-CF₃, and the combination of (R¹)ₘ and (R²)ₙ in each case corresponds to a combination no. of Table B selected from IA-469 to IA-1459.

### Table 65

Compounds of the formula IA wherein Q denotes Q-5, (R³)ₚ is 4-CH(CF₃)₂, and the combination of (R¹)ₘ and (R²)ₙ in each case corresponds to a combination no. of Table B selected from IA-469 to IA-1459.

### Table 66

Compounds of the formula IA wherein Q denotes Q-5, (R³)ₚ is 4-CF(CF₃)₂, and the combination of (R¹)ₘ and (R²)ₙ in each case corresponds to a combination no. of Table B selected from IA-469 to IA-1459.

### Table 67

Compounds of the formula IA wherein Q denotes Q-5, (R³)ₚ is 4-C(OH)(CF₃)₂, and the combination of (R¹)ₘ and (R²)ₙ in each case corresponds to a combination no. of Table B selected from IA-469 to IA-1459.

### Table 68

Compounds of the formula IA wherein Q denotes Q-5, (R³)ₚ is 4-OCF₃, and the combination of (R¹)ₘ and (R²)ₙ in each case corresponds to a combination no. of Table B selected from IA-469 to IA-1459.

### Table 69

Compounds of the formula IA wherein Q denotes Q-5, (R³)ₚ is 4-OCHF₂, and the combination of (R¹)ₘ and (R²)ₙ in each case corresponds to a combination no. of Table B selected from IA-469 to IA-1459.

### Table 70

Compounds of the formula IA wherein Q denotes Q-5, (R³)ₚ is 4-SCF₃, and the combination of (R¹)ₘ and (R²)ₙ in each case corresponds to a combination no. of Table B selected from IA-469 to IA-1459.

### Table 71

Compounds of the formula IA wherein Q denotes Q-5, (R³)ₚ is 4-SO₂CF₃, and the combination of (R¹)ₘ and (R²)ₙ in each case corresponds to a combination no. of Table B selected from IA-469 to IA-1459.

### Table 72

Compounds of the formula IA wherein Q denotes Q-5, (R³)ₚ is 4-CN, and the combination of (R¹)ₘ and (R²)ₙ in each case corresponds to a combination no. of Table B selected from IA-469 to IA-1459.

### Table 73

Compounds of the formula IA wherein Q denotes Q-5, (R³)ₚ is 4-SCH₃, and the combination of (R¹)ₘ and (R²)ₙ in each case corresponds to a combination no. of Table B selected from IA-469 to IA-1459.

### Table 74

Compounds of the formula IA wherein Q denotes Q-5, (R³)ₚ is 4-SO₂CH₃, and the combination of (R¹)ₘ and (R²)ₙ in each case corresponds to a combination no. of Table B selected from IA-469 to IA-1459.

### Table 75

Compounds of the formula IA wherein Q denotes Q-5, (R³)ₚ is 4-OCF₂CF₃, and the combination of (R¹)ₘ and (R²)ₙ in each case corresponds to a combination no. of Table B selected from IA-469 to IA-1459.

### Table 76

Compounds of the formula IA wherein Q denotes Q-5, (R³)ₚ is 4-OCH₂CHCH₂, and the combination of (R¹)ₘ and (R²)ₙ in each case corresponds to a combination no. of Table B selected from IA-469 to IA-1459.

### Table 77

Compounds of the formula IA wherein Q denotes Q-5, (R³)ₚ is 4-CH₂CCH, and the combination of (R¹)ₘ and (R²)ₙ in each case corresponds to a combination no. of Table B selected from IA-469 to IA-1459.

### Table 78

Compounds of the formula IA wherein Q denotes Q-5, (R³)ₚ is 4-CH₂CF₃, and the combination of (R¹)ₘ and (R²)ₙ in each case corresponds to a combination no. of Table B selected from IA-469 to IA-1459.

### Table 79

Compounds of the formula IA wherein Q denotes Q-5, (R³)ₚ is 4-CH₂CH₂OCH₃, and the combination of (R¹)ₘ and (R²)ₙ in each case corresponds to a combination no. of Table B selected from IA-469 to IA-1459.

### Table 80

Compounds of the formula IA wherein Q denotes Q-5, (R³)ₚ is 4-OC(CH₃)₃, and the combination of (R¹)ₘ and (R²)ₙ in each case corresponds to a combination no. of Table B selected from IA-469 to IA-1459.

### Table 81

Compounds of the formula IA wherein Q denotes Q-5, (R³)ₚ is 4-OCH₂-cyclo-C₃H₅, and the combination of (R¹)ₘ and (R²)ₙ in each case corresponds to a combination no. of Table B selected from IA-469 to IA-1459.

### Table 82

Compounds of the formula IA wherein Q denotes Q-5, (R³)ₚ is 5-F, and the combination of (R¹)ₘ and (R²)ₙ in each case corresponds to a combination no. of Table B selected from IA-1 to IA-702 and IA-1379 to IA-1459.

### Table 83

Compounds of the formula IA wherein Q denotes Q-5, (R³)ₚ is 5-Cl, and the combination of (R¹)ₘ and (R²)ₙ in each case corresponds to a combination no. of Table B selected from IA-1 to IA-702 and IA-1379 to IA-1459.

### Table 84

Compounds of the formula IA wherein Q denotes Q-5, (R³)ₚ is 5-Br, and the combination of (R¹)ₘ and (R²)ₙ in each case corresponds to a combination no. of Table B selected from IA-1 to IA-702 and IA-1379 to IA-1459.

### Table 85

Compounds of the formula IA wherein Q denotes Q-5, (R³)ₚ is 5-CH₃, and the combination of (R¹)ₘ and (R²)ₙ in each case corresponds to a combination no. of Table B selected from IA-1 to IA-702 and IA-1379 to IA-1459.

### Table 86

Compounds of the formula IA wherein Q denotes Q-5, (R³)ₚ is 5-OCH₃, and the combination of (R¹)ₘ and (R²)ₙ in each case corresponds to a combination no. of Table B selected from IA-1 to IA-702 and IA-1379 to IA-1459.

### Table 87

Compounds of the formula IA wherein Q denotes Q-5, (R³)ₚ is 5-CF₃, and the combination of (R¹)ₘ and (R²)ₙ in each case corresponds to a combination no. of Table B selected from IA-1 to IA-702 and IA-1379 to IA-1459.

### Table 88

Compounds of the formula IA wherein Q denotes Q-5, (R³)ₚ is 5-OCF₃, and the combination of (R¹)ₘ and (R²)ₙ in each case corresponds to a combination no. of Table B selected from IA-1 to IA-702 and IA-1379 to IA-1459.

### Table 89

Compounds of the formula IA wherein Q denotes Q-5, (R³)ₚ is 5-SCH₃, and the combination of (R¹)ₘ and (R²)ₙ in each case corresponds to a combination no. of Table B selected from IA-1 to IA-702 and IA-1379 to IA-1459.

### Table 90

Compounds of the formula IA wherein Q denotes Q-5, (R³)ₚ is 5-OCHF₂, and the combination of (R¹)ₘ and (R²)ₙ in each case corresponds to a combination no. of Table B selected from IA-1 to IA-702 and IA-1379 to IA-1459.

TABLE B

| No.* | (R¹)ₘ | (R²)ₙ | No.* | (R¹)ₘ | (R²)ₙ |
|---|---|---|---|---|---|
| IA-1 | 2-F | 4-F | IA-2 | 2-F | 4-Cl |
| IA-3 | 2-F | 4-Br | IA-4 | 2-F | 4-CH₃ |
| IA-5 | 2-F | 4-CH₂CH₃ | IA-6 | 2-F | 4-C(CH₃)₃ |
| IA-7 | 2-F | 4-OCH₃ | IA-8 | 2-F | 4-OCH₂CH₃ |
| IA-9 | 2-F | 4-CF₃ | IA-10 | 2-F | 4-CH(CF₃)₂ |
| IA-11 | 2-F | 4-OC(CH₃)₃ | IA-12 | 2-F | 4-CF(CF₃)₂ |
| IA-13 | 2-F | 4-OCF₃ | IA-14 | 2-F | 4-OCHF₂ |
| IA-15 | 2-F | 4-SCF₃ | IA-16 | 2-F | 4-SO₂CF₃ |
| IA-17 | 2-F | 4-CN | IA-18 | 2-F | 4-SCH₃ |
| IA-19 | 2-F | 4-SO₂CH₃ | IA-20 | 2-F | 4-OCF₂CF₃ |
| IA-21 | 2-F | 4- | IA-22 | 2-F | 4- |
| | | OCH₂CHCH₂ | | | OCH₂CH₂OCH₃ |
| IA-23 | 2-F | 4-OCH₂CF₃ | IA-24 | 2-F | 4-OCH₂CCH |
| IA-25 | 2-F | 4-OCH₂- | IA-26 | 2-F | 4-C(OH)(CF₃)₂ |
| | | cyclo-C₃H₅ | | | |
| IA-27 | 2-Cl | 4-F | IA-28 | 2-Cl | 4-Cl |
| IA-29 | 2-Cl | 4-Br | IA-30 | 2-Cl | 4-CH₃ |
| IA-31 | 2-Cl | 4-CH₂CH₃ | IA-32 | 2-Cl | 4-C(CH₃)₃ |
| IA-33 | 2-Cl | 4-OCH₃ | IA-34 | 2-Cl | 4-OCH₂CH₃ |
| IA-35 | 2-Cl | 4-CF₃ | IA-36 | 2-Cl | 4-CH(CF₃)₂ |
| IA-37 | 2-Cl | 4-OC(CH₃)₃ | IA-38 | 2-Cl | 4-CF(CF₃)₂ |
| IA-39 | 2-Cl | 4-OCF₃ | IA-40 | 2-Cl | 4-OCHF₂ |
| IA-41 | 2-Cl | 4-SCF₃ | IA-42 | 2-Cl | 4-SO₂CF₃ |
| IA-43 | 2-Cl | 4-CN | IA-44 | 2-Cl | 4-SCH₃ |
| IA-45 | 2-Cl | 4-SO₂CH₃ | IA-46 | 2-Cl | 4-OCF₂CF₃ |
| IA-47 | 2-Cl | 4- | IA-48 | 2-Cl | 4- |
| | | OCH₂CHCH₂ | | | OCH₂CH₂OCH₃ |
| IA-49 | 2-Cl | 4-OCH₂CF₃ | IA-50 | 2-Cl | 4-OCH₂CCH |
| IA-51 | 2-Cl | 4-OCH₂- | IA-52 | 2-Cl | 4-C(OH)(CF₃)₂ |
| | | cyclo-C₃H₅ | | | |
| IA-53 | 2-Br | 4-F | IA-54 | 2-Br | 4-Cl |
| IA-55 | 2-Br | 4-Br | IA-56 | 2-Cl | 4-CH₃ |
| IA-57 | 2-Br | 4-CH₂CH₃ | IA-58 | 2-Br | 4-C(CH₃)₃ |
| IA-59 | 2-Br | 4-OCH₃ | IA-60 | 2-Br | 4-OCH₂CH₃ |
| IA-61 | 2-Br | 4-CF₃ | IA-62 | 2-Br | 4-CH(CF₃)₂ |
| IA-63 | 2-Br | 4-OC(CH₃)₃ | IA-64 | 2-Br | 4-CF(CF₃)₂ |
| IA-65 | 2-Br | 4-OCF₃ | IA-66 | 2-Br | 4-OCHF₂ |
| IA-67 | 2-Br | 4-SCF₃ | IA-68 | 2-Br | 4-SO₂CF₃ |
| IA-69 | 2-Br | 4-CN | IA-70 | 2-Br | 4-SCH₃ |
| IA-71 | 2-Br | 4-SO₂CH₃ | IA-72 | 2-Br | 4-OCF₂CF₃ |
| IA-73 | 2-Br | 4- | IA-74 | 2-Br | 4- |
| | | OCH₂CHCH₂ | | | OCH₂CH₂OCH₃ |
| IA-75 | 2-Br | 4-OCH₂CF₃ | IA-76 | 2-Br | 4-OCH₂CCH |
| IA-77 | 2-Br | 4-OCH₂- | IA-78 | 2-Br | 4-C(OH)(CF₃)₂ |
| | | cyclo-C₃H₅ | | | |
| IA-79 | 2-CH₃ | 4-F | IA-80 | 2-CH₃ | 4-Cl |
| IA-81 | 2-CH₃ | 4-Br | IA-82 | 2-CH₃ | 4-CH₃ |
| IA-83 | 2-CH₃ | 4-CH₂CH₃ | IA-84 | 2-CH₃ | 4-C(CH₃)₃ |
| IA-85 | 2-CH₃ | 4-OCH₃ | IA-86 | 2-CH₃ | 4-OCH₂CH₃ |
| IA-87 | 2-CH₃ | 4-CF₃ | IA-88 | 2-CH₃ | 4-CH(CF₃)₂ |
| IA-89 | 2-CH₃ | 4-OC(CH₃)₃ | IA-90 | 2-CH₃ | 4-CF(CF₃)₂ |
| IA-91 | 2-CH₃ | 4-OCF₃ | IA-92 | 2-CH₃ | 4-OCHF₂ |
| IA-93 | 2-CH₃ | 4-SCF₃ | IA-94 | 2-CH₃ | 4-SO₂CF₃ |
| IA-95 | 2-CH₃ | 4-CN | IA-96 | 2-CH₃ | 4-SCH₃ |
| IA-97 | 2-CH₃ | 4-SO₂CH₃ | IA-98 | 2-CH₃ | 4-OCF₂CF₃ |
| IA-99 | 2-CH₃ | 4- | IA-100 | 2-CH₃ | 4- |
| | | OCH₂CHCH₂ | | | OCH₂CH₂OCH₃ |
| IA-101 | 2-CH₃ | 4-OCH₂CF₃ | IA-102 | 2-CH₃ | 4-OCH₂CCH |
| IA-103 | 2-CH₃ | 4-OCH₂- | IA-104 | 2-CH₃ | 4-C(OH)(CF₃)₂ |
| | | cyclo-C₃H₅ | | | |
| IA-105 | 2-OCH₃ | 4-F | IA-106 | 2-OCH₃ | 4-Cl |
| IA-107 | 2-OCH₃ | 4-Br | IA-108 | 2-OCH₃ | 4-CH₃ |
| IA-109 | 2-OCH₃ | 4-CH₂CH₃ | IA-110 | 2-OCH₃ | 4-C(CH₃)₃ |
| IA-111 | 2-OCH₃ | 4-OCH₃ | IA-112 | 2-OCH₃ | 4-OCH₂CH₃ |
| IA-113 | 2-OCH₃ | 4-CF₃ | IA-114 | 2-OCH₃ | 4-CH(CF₃)₂ |
| IA-115 | 2-OCH₃ | 4-OC(CH₃)₃ | IA-116 | 2-OCH₃ | 4-CF(CF₃)₂ |
| IA-117 | 2-OCH₃ | 4-OCF₃ | IA-118 | 2-OCH₃ | 4-OCHF₂ |
| IA-119 | 2-OCH₃ | 4-SCF₃ | IA-120 | 2-OCH₃ | 4-SO₂CF₃ |
| IA-121 | 2-OCH₃ | 4-CN | IA-122 | 2-OCH₃ | 4-SCH₃ |
| IA-123 | 2-OCH₃ | 4-SO₂CH₃ | IA-124 | 2-OCH₃ | 4-OCF₂CF₃ |
| IA-125 | 2-OCH₃ | 4- | IA-126 | 2-OCH₃ | 4- |
| | | OCH₂CHCH₂ | | | OCH₂CH₂OCH₃ |
| IA-127 | 2-OCH₃ | 4-OCH₂CF₃ | IA-128 | 2-OCH₃ | 4-OCH₂CCH |
| IA-129 | 2-OCH₃ | 4-OCH₂- | IA-130 | 2-OCH₃ | 4-C(OH)(CF₃)₂ |
| | | cyclo-C₃H₅ | | | |
| IA-131 | 2-CF₃ | 4-F | IA-132 | 2-CF₃ | 4-Cl |
| IA-133 | 2-CF₃ | 4-Br | IA-134 | 2-CF₃ | 4-CH₃ |
| IA-135 | 2-CF₃ | 4-CH₂CH₃ | IA-136 | 2-CF₃ | 4-C(CH₃)₃ |
| IA-137 | 2-CF₃ | 4-OCH₃ | IA-138 | 2-CF₃ | 4-OCH₂CH₃ |
| IA-139 | 2-CF₃ | 4-CF₃ | IA-140 | 2-CF₃ | 4-CH(CF₃)₂ |
| IA-141 | 2-CF₃ | 4-OC(CH₃)₃ | IA-142 | 2-CF₃ | 4-CF(CF₃)₂ |
| IA-143 | 2-CF₃ | 4-OCF₃ | IA-144 | 2-CF₃ | 4-OCHF₂ |
| IA-145 | 2-CF₃ | 4-SCF₃ | IA-146 | 2-CF₃ | 4-SO₂CF₃ |
| IA-147 | 2-CF₃ | 4-CN | IA-148 | 2-CF₃ | 4-SCH₃ |
| IA-149 | 2-CF₃ | 4-SO₂CH₃ | IA-150 | 2-CF₃ | 4-OCF₂CF₃ |
| IA-151 | 2-CF₃ | 4- | IA-152 | 2-CF₃ | 4- |
| | | OCH₂CHCH₂ | | | OCH₂CH₂OCH₃ |
| IA-153 | 2-CF₃ | 4-OCH₂CF₃ | IA-154 | 2-CF₃ | 4-OCH₂CCH |
| IA-155 | 2-CF₃ | 4-OCH₂- | IA-156 | 2-CF₃ | 4-C(OH)(CF₃)₂ |
| | | cyclo-C₃H₅ | | | |
| IA-157 | 2-OCF₃ | 4-F | IA-158 | 2-OCF₃ | 4-Cl |
| IA-159 | 2-OCF₃ | 4-Br | IA-160 | 2-OCF₃ | 4-CH₃ |
| IA-161 | 2-OCF₃ | 4-CH₂CH₃ | IA-162 | 2-OCF₃ | 4-C(CH₃)₃ |
| IA-163 | 2-OCF₃ | 4-OCH₃ | IA-164 | 2-OCF₃ | 4-OCH₂CH₃ |
| IA-165 | 2-OCF₃ | 4-CF₃ | IA-166 | 2-OCF₃ | 4-CH(CF₃)₂ |
| IA-167 | 2-OCF₃ | 4-OC(CH₃)₃ | IA-168 | 2-OCF₃ | 4-CF(CF₃)₂ |
| IA-169 | 2-OCF₃ | 4-OCF₃ | IA-170 | 2-OCF₃ | 4-OCHF₂ |
| IA-171 | 2-OCF₃ | 4-SCF₃ | IA-172 | 2-OCF₃ | 4-SO₂CF₃ |
| IA-173 | 2-OCF₃ | 4-CN | IA-174 | 2-OCF₃ | 4-SCH₃ |
| IA-175 | 2-OCF₃ | 4-SO₂CH₃ | IA-176 | 2-OCF₃ | 4-OCF₂CF₃ |
| IA-177 | 2-OCF₃ | 4- | IA-178 | 2-OCF₃ | 4- |
| | | OCH₂CHCH₂ | | | OCH₂CH₂OCH₃ |
| IA-179 | 2-OCF₃ | 4-OCH₂CF₃ | IA-180 | 2-OCF₃ | 4-OCH₂CCH |
| IA-181 | 2-OCF₃ | 4-OCH₂- | IA-182 | 2-OCF₃ | 4-C(OH)(CF₃)₂ |
| | | cyclo-C₃H₅ | | | |
| IA-183 | 2-SCH₃ | 4-F | IA-184 | 2-SCH₃ | 4-Cl |
| IA-185 | 2-SCH₃ | 4-Br | IA-186 | 2-SCH₃ | 4-CH₃ |
| IA-187 | 2-SCH₃ | 4-CH₂CH₃ | IA-188 | 2-SCH₃ | 4-C(CH₃)₃ |
| IA-189 | 2-SCH₃ | 4-OCH₃ | IA-190 | 2-SCH₃ | 4-OCH₂CH₃ |
| IA-191 | 2-SCH₃ | 4-CF₃ | IA-192 | 2-SCH₃ | 4-CH(CF₃)₂ |
| IA-193 | 2-SCH₃ | 4-OC(CH₃)₃ | IA-194 | 2-SCH₃ | 4-CF(CF₃)₂ |
| IA-195 | 2-SCH₃ | 4-OCF₃ | IA-196 | 2-SCH₃ | 4-OCHF₂ |
| IA-197 | 2-SCH₃ | 4-SCF₃ | IA-198 | 2-SCH₃ | 4-SO₂CF₃ |
| IA-199 | 2-SCH₃ | 4-CN | IA-200 | 2-SCH₃ | 4-SCH₃ |
| IA-201 | 2-SCH₃ | 4-SO₂CH₃ | IA-202 | 2-SCH₃ | 4-OCF₂CF₃ |
| IA-203 | 2-SCH₃ | 4- | IA-204 | 2-SCH₃ | 4- |
| | | OCH₂CHCH₂ | | | OCH₂CH₂OCH₃ |
| IA-205 | 2-SCH₃ | 4-OCH₂CF₃ | IA-206 | 2-SCH₃ | 4-OCH₂CCH |
| IA-207 | 2-SCH₃ | 4-OCH₂- | IA-208 | 2-SCH₃ | 4-C(OH)(CF₃)₂ |
| | | cyclo-C₃H₅ | | | |
| IA-209 | 2-OCHF₂ | 4-F | IA-210 | 2-OCHF₂ | 4-Cl |
| IA-211 | 2-OCHF₂ | 4-Br | IA-212 | 2-OCHF₂ | 4-CH₃ |
| IA-213 | 2-OCHF₂ | 4-CH₂CH₃ | IA-214 | 2-OCHF₂ | 4-C(CH₃)₃ |
| IA-215 | 2-OCHF₂ | 4-OCH₃ | IA-216 | 2-OCHF₂ | 4-OCH₂CH₃ |
| IA-217 | 2-OCHF₂ | 4-CF₃ | IA-218 | 2-OCHF₂ | 4-CH(CF₃)₂ |
| IA-219 | 2-OCHF₂ | 4-OC(CH₃)₃ | IA-220 | 2-OCHF₂ | 4-CF(CF₃)₂ |
| IA-221 | 2-OCHF₂ | 4-OCF₃ | IA-222 | 2-OCHF₂ | 4-OCHF₂ |
| IA-223 | 2-OCHF₂ | 4-SCF₃ | IA-224 | 2-OCHF₂ | 4-SO₂CF₃ |
| IA-225 | 2-OCHF₂ | 4-CN | IA-226 | 2-OCHF₂ | 4-SCH₃ |
| IA-227 | 2-OCHF₂ | 4-SO₂CH₃ | IA-228 | 2-OCHF₂ | 4-OCF₂CF₃ |
| IA-229 | 2-OCHF₂ | 4- | IA-230 | 2-OCHF₂ | 4- |
| | | OCH₂CHCH₂ | | | OCH₂CH₂OCH₃ |
| IA-231 | 2-OCHF₂ | 4-OCH₂CF₃ | IA-232 | 2-OCHF₂ | 4-OCH₂CCH |
| IA-233 | 2-OCHF₂ | 4-OCH₂- | IA-234 | 2-OCHF₂ | 4-C(OH)(CF₃)₂ |
| | | cyclo-C₃H₅ | | | |
| IA-235 | 3-F | 4-F | IA-236 | 3-F | 4-Cl |
| IA-237 | 3-F | 4-Br | IA-238 | 3-F | 4-CH₃ |
| IA-239 | 3-F | 4-CH₂CH₃ | IA-240 | 3-F | 4-C(CH₃)₃ |
| IA-241 | 3-F | 4-OCH₃ | IA-242 | 3-F | 4-OCH₂CH₃ |
| IA-243 | 3-F | 4-CF₃ | IA-244 | 3-F | 4-CH(CF₃)₂ |
| IA-245 | 3-F | 4-OC(CH₃)₃ | IA-246 | 3-F | 4-CF(CF₃)₂ |
| IA-247 | 3-F | 4-OCF₃ | IA-248 | 3-F | 4-OCHF₂ |
| IA-249 | 3-F | 4-SCF₃ | IA-250 | 3-F | 4-SO₂CF₃ |
| IA-251 | 3-F | 4-CN | IA-252 | 3-F | 4-SCH₃ |
| IA-253 | 3-F | 4-SO₂CH₃ | IA-254 | 3-F | 4-OCF₂CF₃ |
| IA-255 | 3-F | 4- | IA-256 | 3-F | 4- |
| | | OCH₂CHCH₂ | | | OCH₂CH₂OCH₃ |
| IA-257 | 3-F | 4-OCH₂CF₃ | IA-258 | 3-F | 4-OCH₂CCH |
| IA-259 | 3-F | 4-OCH₂- | IA-260 | 3-F | 4-C(OH)(CF₃)₂ |
| | | cyclo-C₃H₅ | | | |
| IA-261 | 3-Cl | 4-F | IA-262 | 3-Cl | 4-Cl |
| IA-263 | 3-Cl | 4-Br | IA-264 | 3-Cl | 4-CH₃ |
| IA-265 | 3-Cl | 4-CH₂CH₃ | IA-266 | 3-Cl | 4-C(CH₃)₃ |
| IA-267 | 3-Cl | 4-OCH₃ | IA-268 | 3-Cl | 4-OCH₂CH₃ |
| IA-269 | 3-Cl | 4-CF₃ | IA-270 | 3-Cl | 4-CH(CF₃)₂ |
| IA-271 | 3-Cl | 4-OC(CH₃)₃ | IA-272 | 3-Cl | 4-CF(CF₃)₂ |
| IA-273 | 3-Cl | 4-OCF₃ | IA-274 | 3-Cl | 4-OCHF₂ |
| IA-275 | 3-Cl | 4-SCF₃ | IA-276 | 3-Cl | 4-SO₂CF₃ |
| IA-277 | 3-Cl | 4-CN | IA-278 | 3-Cl | 4-SCH₃ |
| IA-279 | 3-Cl | 4-SO₂CH₃ | IA-280 | 3-Cl | 4-OCF₂CF₃ |
| IA-281 | 3-Cl | 4- | IA-282 | 3-Cl | 4- |
| | | OCH₂CHCH₂ | | | OCH₂CH₂OCH₃ |
| IA-283 | 3-Cl | 4-OCH₂CF₃ | IA-284 | 3-Cl | 4-OCH₂CCH |
| IA-285 | 3-Cl | 4-OCH₂- | IA-286 | 3-Cl | 4-C(OH)(CF₃)₂ |
| | | cyclo-C₃H₅ | | | |
| IA-287 | 3-Br | 4-F | IA-288 | 3-Br | 4-Cl |
| IA-289 | 3-Br | 4-Br | IA-290 | 3-Cl | 4-CH₃ |
| IA-291 | 3-Br | 4-CH₂CH₃ | IA-292 | 3-Br | 4-C(CH₃)₃ |
| IA-293 | 3-Br | 4-OCH₃ | IA-294 | 3-Br | 4-OCH₂CH₃ |
| IA-295 | 3-Br | 4-CF₃ | IA-296 | 3-Br | 4-CH(CF₃)₂ |
| IA-297 | 3-Br | 4-OC(CH₃)₃ | IA-298 | 3-Br | 4-CF(CF₃)₂ |
| IA-299 | 3-Br | 4-OCF₃ | IA-300 | 3-Br | 4-OCHF₂ |
| IA-301 | 3-Br | 4-SCF₃ | IA-302 | 3-Br | 4-SO₂CF₃ |
| IA-303 | 3-Br | 4-CN | IA-304 | 3-Br | 4-SCH₃ |
| IA-305 | 3-Br | 4-SO₂CH₃ | IA-306 | 3-Br | 4-OCF₂CF₃ |
| IA-307 | 3-Br | 4- | IA-308 | 3-Br | 4- |
| | | OCH₂CHCH₂ | | | OCH₂CH₂OCH₃ |
| IA-309 | 3-Br | 4-OCH₂CF₃ | IA-310 | 3-Br | 4-OCH₂CCH |
| IA-311 | 3-Br | 4-OCH₂- | IA-312 | 3-Br | 4-C(OH)(CF₃)₂ |
| | | cyclo-C₃H₅ | | | |
| IA-313 | 3-CH₃ | 4-F | IA-314 | 3-CH₃ | 4-Cl |
| IA-315 | 3-CH₃ | 4-Br | IA-316 | 3-CH₃ | 4-CH₃ |
| IA-317 | 3-CH₃ | 4-CH₂CH₃ | IA-318 | 3-CH₃ | 4-C(CH₃)₃ |
| IA-319 | 3-CH₃ | 4-OCH₃ | IA-320 | 3-CH₃ | 4-OCH₂CH₃ |
| IA-321 | 3-CH₃ | 4-CF₃ | IA-322 | 3-CH₃ | 4-CH(CF₃)₂ |
| IA-323 | 3-CH₃ | 4-OC(CH₃)₃ | IA-324 | 3-CH₃ | 4-CF(CF₃)₂ |
| IA-325 | 3-CH₃ | 4-OCF₃ | IA-326 | 3-CH₃ | 4-OCHF₂ |
| IA-327 | 3-CH₃ | 4-SCF₃ | IA-328 | 3-CH₃ | 4-SO₂CF₃ |
| IA-329 | 3-CH₃ | 4-CN | IA-330 | 3-CH₃ | 4-SCH₃ |
| IA-331 | 3-CH₃ | 4-SO₂CH₃ | IA-332 | 3-CH₃ | 4-OCF₂CF₃ |
| IA-333 | 3-CH₃ | 4- | IA-334 | 3-CH₃ | 4- |
| | | OCH₂CHCH₂ | | | OCH₂CH₂OCH₃ |
| IA-335 | 3-CH₃ | 4-OCH₂CF₃ | IA-336 | 3-CH₃ | 4-OCH₂CCH |
| IA-337 | 3-CH₃ | 4-OCH₂- | IA-338 | 3-CH₃ | 4-C(OH)(CF₃)₂ |
| | | cyclo-C₃H₅ | | | |
| IA-339 | 3-OCH₃ | 4-F | IA-340 | 3-OCH₃ | 4-Cl |
| IA-341 | 3-OCH₃ | 4-Br | IA-342 | 3-OCH₃ | 4-CH₃ |
| IA-343 | 3-OCH₃ | 4-CH₂CH₃ | IA-344 | 3-OCH₃ | 4-C(CH₃)₃ |
| IA-345 | 3-OCH₃ | 4-OCH₃ | IA-346 | 3-OCH₃ | 4-OCH₂CH₃ |
| IA-347 | 3-OCH₃ | 4-CF₃ | IA-348 | 3-OCH₃ | 4-CH(CF₃)₂ |
| IA-349 | 3-OCH₃ | 4-OC(CH₃)₃ | IA-350 | 3-OCH₃ | 4-CF(CF₃)₂ |
| IA-351 | 3-OCH₃ | 4-OCF₃ | IA-352 | 3-OCH₃ | 4-OCHF₂ |
| IA-353 | 3-OCH₃ | 4-SCF₃ | IA-354 | 3-OCH₃ | 4-SO₂CF₃ |
| IA-355 | 3-OCH₃ | 4-CN | IA-356 | 3-OCH₃ | 4-SCH₃ |
| IA-357 | 3-OCH₃ | 4-SO₂CH₃ | IA-358 | 3-OCH₃ | 4-OCF₂CF₃ |
| IA-359 | 3-OCH₃ | 4- | IA-360 | 3-OCH₃ | 4- |
| | | OCH₂CHCH₂ | | | OCH₂CH₂OCH₃ |
| IA-361 | 3-OCH₃ | 4-OCH₂CF₃ | IA-362 | 3-OCH₃ | 4-OCH₂CCH |
| IA-363 | 3-OCH₃ | 4-OCH₂- | IA-364 | 3-OCH₃ | 4-C(OH)(CF₃)₂ |
| | | cyclo-C₃H₅ | | | |
| IA-365 | 3-CF₃ | 4-F | IA-366 | 3-CF₃ | 4-Cl |
| IA-367 | 3-CF₃ | 4-Br | IA-368 | 3-CF₃ | 4-CH₃ |
| IA-369 | 3-CF₃ | 4-CH₂CH₃ | IA-370 | 3-CF₃ | 4-C(CH₃)₃ |
| IA-371 | 3-CF₃ | 4-OCH₃ | IA-372 | 3-CF₃ | 4-OCH₂CH₃ |
| IA-373 | 3-CF₃ | 4-CF₃ | IA-374 | 3-CF₃ | 4-CH(CF₃)₂ |
| IA-375 | 3-CF₃ | 4-OC(CH₃)₃ | IA-376 | 3-CF₃ | 4-CF(CF₃)₂ |
| IA-377 | 3-CF₃ | 4-OCF₃ | IA-378 | 3-CF₃ | 4-OCHF₂ |
| IA-379 | 3-CF₃ | 4-SCF₃ | IA-380 | 3-CF₃ | 4-SO₂CF₃ |
| IA-381 | 3-CF₃ | 4-CN | IA-382 | 3-CF₃ | 4-SCH₃ |
| IA-383 | 3-CF₃ | 4-SO₂CH₃ | IA-384 | 3-CF₃ | 4-OCF₂CF₃ |
| IA-385 | 3-CF₃ | 4- | IA-386 | 3-CF₃ | 4- |
| | | OCH₂CHCH₂ | | | OCH₂CH₂OCH₃ |
| IA-387 | 3-CF₃ | 4-OCH₂CF₃ | IA-388 | 3-CF₃ | 4-OCH₂CCH |
| IA-389 | 3-CF₃ | 4-OCH₂- | IA-390 | 3-CF₃ | 4-C(OH)(CF₃)₂ |
| | | cyclo-C₃H₅ | | | |
| IA-391 | 3-OCF₃ | 4-F | IA-392 | 3-OCF₃ | 4-Cl |
| IA-393 | 3-OCF₃ | 4-Br | IA-394 | 3-OCF₃ | 4-CH₃ |
| IA-395 | 3-OCF₃ | 4-CH₂CH₃ | IA-396 | 3-OCF₃ | 4-C(CH₃)₃ |
| IA-397 | 3-OCF₃ | 4-OCH₃ | IA-398 | 3-OCF₃ | 4-OCH₂CH₃ |
| IA-399 | 3-OCF₃ | 4-CF₃ | IA-400 | 3-OCF₃ | 4-CH(CF₃)₂ |
| IA-401 | 3-OCF₃ | 4-OC(CH₃)₃ | IA-402 | 3-OCF₃ | 4-CF(CF₃)₂ |
| IA-403 | 3-OCF₃ | 4-OCF₃ | IA-404 | 3-OCF₃ | 4-OCHF₂ |
| IA-405 | 3-OCF₃ | 4-SCF₃ | IA-406 | 3-OCF₃ | 4-SO₂CF₃ |
| IA-407 | 3-OCF₃ | 4-CN | IA-408 | 3-OCF₃ | 4-SCH₃ |
| IA-409 | 3-OCF₃ | 4-SO₂CH₃ | IA-410 | 3-OCF₃ | 4-OCF₂CF₃ |
| IA-411 | 3-OCF₃ | 4- | IA-412 | 3-OCF₃ | 4- |
| | | OCH₂CHCH₂ | | | OCH₂CH₂OCH₃ |
| IA-413 | 3-OCF₃ | 4-OCH₂CF₃ | IA-414 | 3-OCF₃ | 4-OCH₂CCH |
| IA-415 | 3-OCF₃ | 4-OCH₂- | IA-416 | 3-OCF₃ | 4-C(OH)(CF₃)₂ |
| | | cyclo-C₃H₅ | | | |
| IA-417 | 3-SCH₃ | 4-F | IA-418 | 3-SCH₃ | 4-Cl |
| IA-419 | 3-SCH₃ | 4-Br | IA-420 | 3-SCH₃ | 4-CH₃ |
| IA-421 | 3-SCH₃ | 4-CH₂CH₃ | IA-422 | 3-SCH₃ | 4-C(CH₃)₃ |
| IA-423 | 3-SCH₃ | 4-OCH₃ | IA-424 | 3-SCH₃ | 4-OCH₂CH₃ |
| IA-425 | 3-SCH₃ | 4-CF₃ | IA-426 | 3-SCH₃ | 4-CH(CF₃)₂ |
| IA-427 | 3-SCH₃ | 4-OC(CH₃)₃ | IA-428 | 3-SCH₃ | 4-CF(CF₃)₂ |
| IA-429 | 3-SCH₃ | 4-OCF₃ | IA-430 | 3-SCH₃ | 4-OCHF₂ |
| IA-431 | 3-SCH₃ | 4-SCF₃ | IA-432 | 3-SCH₃ | 4-SO₂CF₃ |
| IA-433 | 3-SCH₃ | 4-CN | IA-434 | 3-SCH₃ | 4-SCH₃ |
| IA-435 | 3-SCH₃ | 4-SO₂CH₃ | IA-436 | 3-SCH₃ | 4-OCF₂CF₃ |
| IA-437 | 3-SCH₃ | 4- | IA-438 | 3-SCH₃ | 4- |
| | | OCH₂CHCH₂ | | | OCH₂CH₂OCH₃ |
| IA-439 | 3-SCH₃ | 4-OCH₂CF₃ | IA-440 | 3-SCH₃ | 4-OCH₂CCH |
| IA-441 | 3-SCH₃ | 4-OCH₂- | IA-442 | 3-SCH₃ | 4-C(OH)(CF₃)₂ |
| | | cyclo-C₃H₅ | | | |
| IA-443 | 3-OCHF₂ | 4-F | IA-444 | 3-OCHF₂ | 4-Cl |
| IA-445 | 3-OCHF₂ | 4-Br | IA-446 | 3-OCHF₂ | 4-CH₃ |
| IA-447 | 3-OCHF₂ | 4-CH₂CH₃ | IA-448 | 3-OCHF₂ | 4-C(CH₃)₃ |
| IA-449 | 3-OCHF₂ | 4-OCH₃ | IA-450 | 3-OCHF₂ | 4-OCH₂CH₃ |
| IA-451 | 3-OCHF₂ | 4-CF₃ | IA-452 | 3-OCHF₂ | 4-CH(CF₃)₂ |
| IA-453 | 3-OCHF₂ | 4-OC(CH₃)₃ | IA-454 | 3-OCHF₂ | 4-CF(CF₃)₂ |
| IA-455 | 3-OCHF₂ | 4-OCF₃ | IA-456 | 3-OCHF₂ | 4-OCHF₂ |
| IA-457 | 3-OCHF₂ | 4-SCF₃ | IA-458 | 3-OCHF₂ | 4-SO₂CF₃ |
| IA-459 | 3-OCHF₂ | 4-CN | IA-460 | 3-OCHF₂ | 4-SCH₃ |
| IA-461 | 3-OCHF₂ | 4-SO₂CH₃ | IA-462 | 3-OCHF₂ | 4-OCF₂CF₃ |
| IA-463 | 3-OCHF₂ | 4- | IA-464 | 3-OCHF₂ | 4- |
| | | OCH₂CHCH₂ | | | OCH₂CH₂OCH₃ |
| IA-465 | 3-OCHF₂ | 4-OCH₂CF₃ | IA-466 | 3-OCHF₂ | 4-OCH₂CCH |
| IA-467 | 3-OCHF₂ | 4-OCH₂- | IA-468 | 3-OCHF₂ | 4-C(OH)(CF₃)₂ |
| | | cyclo-C₃H₅ | | | |
| IA-469 | 2-F | 3-F | IA-470 | 2-F | 3-Cl |
| IA-471 | 2-F | 3-Br | IA-472 | 2-F | 3-CH₃ |
| IA-473 | 2-F | 3-CH₂CH₃ | IA-474 | 2-F | 3-C(CH₃)₃ |
| IA-475 | 2-F | 3-OCH₃ | IA-476 | 2-F | 3-OCH₂CH₃ |
| IA-477 | 2-F | 3-CF₃ | IA-478 | 2-F | 3-CH(CF₃)₂ |
| IA-479 | 2-F | 3-OC(CH₃)₃ | IA-480 | 2-F | 3-CF(CF₃)₂ |
| IA-481 | 2-F | 3-OCF₃ | IA-482 | 2-F | 3-OCHF₂ |
| IA-483 | 2-F | 3-SCF₃ | IA-484 | 2-F | 3-SO₂CF₃ |
| IA-485 | 2-F | 3-CN | IA-486 | 2-F | 3-SCH₃ |
| IA-487 | 2-F | 3-SO₂CH₃ | IA-488 | 2-F | 3-OCF₂CF₃ |
| IA-489 | 2-F | 3- | IA-490 | 2-F | 3- |
| | | OCH₂CHCH₂ | | | OCH₂CH₂OCH₃ |
| IA-491 | 2-F | 3-OCH₂CF₃ | IA-492 | 2-F | 3-OCH₂CCH |
| IA-493 | 2-F | 3-OCH₂- | IA-494 | 2-F | 3-C(OH)(CF₃)₂ |
| | | cyclo-C₃H₅ | | | |
| IA-495 | 2-Cl | 3-F | IA-496 | 2-Cl | 3-Cl |
| IA-497 | 2-Cl | 3-Br | IA-498 | 2-Cl | 3-CH₃ |
| IA-499 | 2-Cl | 3-CH₂CH₃ | IA-500 | 2-Cl | 3-C(CH₃)₃ |
| IA-501 | 2-Cl | 3-OCH₃ | IA-502 | 2-Cl | 3-OCH₂CH₃ |
| IA-503 | 2-Cl | 3-CF₃ | IA-504 | 2-Cl | 3-CH(CF₃)₂ |
| IA-505 | 2-Cl | 3-OC(CH₃)₃ | IA-506 | 2-Cl | 3-CF(CF₃)₂ |
| IA-507 | 2-Cl | 3-OCF₃ | IA-508 | 2-Cl | 3-OCHF₂ |
| IA-509 | 2-Cl | 3-SCF₃ | IA-510 | 2-Cl | 3-SO₂CF₃ |
| IA-511 | 2-Cl | 3-CN | IA-512 | 2-Cl | 3-SCH₃ |
| IA-513 | 2-Cl | 3-SO₂CH₃ | IA-514 | 2-Cl | 3-OCF₂CF₃ |
| IA-515 | 2-Cl | 3- | IA-516 | 2-Cl | 3- |
| | | OCH₂CHCH₂ | | | OCH₂CH₂OCH₃ |
| IA-517 | 2-Cl | 3-OCH₂CF₃ | IA-518 | 2-Cl | 3-OCH₂CCH |
| IA-519 | 2-Cl | 3-OCH₂- | IA-520 | 2-Cl | 3-C(OH)(CF₃)₂ |
| | | cyclo-C₃H₅ | | | |
| IA-521 | 2-Br | 3-F | IA-522 | 2-Br | 3-Cl |
| IA-523 | 2-Br | 3-Br | IA-524 | 2-Cl | 3-CH₃ |
| IA-525 | 2-Br | 3-CH₂CH₃ | IA-526 | 2-Br | 3-C(CH₃)₃ |
| IA-527 | 2-Br | 3-OCH₃ | IA-528 | 2-Br | 3-OCH₂CH₃ |
| IA-529 | 2-Br | 3-CF₃ | IA-530 | 2-Br | 3-CH(CF₃)₂ |
| IA-531 | 2-Br | 3-OC(CH₃)₃ | IA-532 | 2-Br | 3-CF(CF₃)₂ |
| IA-533 | 2-Br | 3-OCF₃ | IA-534 | 2-Br | 3-OCHF₂ |
| IA-535 | 2-Br | 3-SCF₃ | IA-536 | 2-Br | 3-SO₂CF₃ |
| IA-537 | 2-Br | 3-CN | IA-538 | 2-Br | 3-SCH₃ |
| IA-539 | 2-Br | 3-SO₂CH₃ | IA-540 | 2-Br | 3-OCF₂CF₃ |
| IA-541 | 2-Br | 3- | IA-542 | 2-Br | 3- |
| | | OCH₂CHCH₂ | | | OCH₂CH₂OCH₃ |
| IA-543 | 2-Br | 3-OCH₂CF₃ | IA-544 | 2-Br | 3-OCH₂CCH |
| IA-545 | 2-Br | 3-OCH₂- | IA-546 | 2-Br | 3-C(OH)(CF₃)₂ |
| | | cyclo-C₃H₅ | | | |
| IA-547 | 2-CH₃ | 3-F | IA-548 | 2-CH₃ | 3-Cl |
| IA-549 | 2-CH₃ | 3-Br | IA-550 | 2-CH₃ | 3-CH₃ |
| IA-551 | 2-CH₃ | 3-CH₂CH₃ | IA-552 | 2-CH₃ | 3-C(CH₃)₃ |
| IA-553 | 2-CH₃ | 3-OCH₃ | IA-554 | 2-CH₃ | 3-OCH₂CH₃ |
| IA-555 | 2-CH₃ | 3-CF₃ | IA-556 | 2-CH₃ | 3-CH(CF₃)₂ |
| IA-557 | 2-CH₃ | 3-OC(CH₃)₃ | IA-558 | 2-CH₃ | 3-CF(CF₃)₂ |
| IA-559 | 2-CH₃ | 3-OCF₃ | IA-560 | 2-CH₃ | 3-OCHF₂ |
| IA-561 | 2-CH₃ | 3-SCF₃ | IA-562 | 2-CH₃ | 3-SO₂CF₃ |
| IA-563 | 2-CH₃ | 3-CN | IA-564 | 2-CH₃ | 3-SCH₃ |
| IA-565 | 2-CH₃ | 3-SO₂CH₃ | IA-566 | 2-CH₃ | 3-OCF₂CF₃ |
| IA-567 | 2-CH₃ | 3- | IA-568 | 2-CH₃ | 3- |
| | | OCH₂CHCH₂ | | | OCH₂CH₂OCH₃ |
| IA-569 | 2-CH₃ | 3-OCH₂CF₃ | IA-570 | 2-CH₃ | 3-OCH₂CCH |
| IA-571 | 2-CH₃ | 3-OCH₂- | IA-572 | 2-CH₃ | 3-C(OH)(CF₃)₂ |
| | | cyclo-C₃H₅ | | | |
| IA-573 | 2-OCH₃ | 3-F | IA-574 | 2-OCH₃ | 3-Cl |
| IA-575 | 2-OCH₃ | 3-Br | IA-576 | 2-OCH₃ | 3-CH₃ |
| IA-577 | 2-OCH₃ | 3-CH₂CH₃ | IA-578 | 2-OCH₃ | 3-C(CH₃)₃ |
| IA-579 | 2-OCH₃ | 3-OCH₃ | IA-580 | 2-OCH₃ | 3-OCH₂CH₃ |
| IA-581 | 2-OCH₃ | 3-CF₃ | IA-582 | 2-OCH₃ | 3-CH(CF₃)₂ |
| IA-583 | 2-OCH₃ | 3-OC(CH₃)₃ | IA-584 | 2-OCH₃ | 3-CF(CF₃)₂ |
| IA-585 | 2-OCH₃ | 3-OCF₃ | IA-586 | 2-OCH₃ | 3-OCHF₂ |
| IA-587 | 2-OCH₃ | 3-SCF₃ | IA-588 | 2-OCH₃ | 3-SO₂CF₃ |
| IA-589 | 2-OCH₃ | 3-CN | IA-590 | 2-OCH₃ | 3-SCH₃ |
| IA-591 | 2-OCH₃ | 3-SO₂CH₃ | IA-592 | 2-OCH₃ | 3-OCF₂CF₃ |
| IA-593 | 2-OCH₃ | 3- | IA-594 | 2-OCH₃ | 3- |
| | | OCH₂CHCH₂ | | | OCH₂CH₂OCH₃ |
| IA-595 | 2-OCH₃ | 3-OCH₂CF₃ | IA-596 | 2-OCH₃ | 3-OCH₂CCH |
| IA-597 | 2-OCH₃ | 3-OCH₂- | IA-598 | 2-OCH₃ | 3-C(OH)(CF₃)₂ |
| | | cyclo-C₃H₅ | | | |
| IA-599 | 2-CF₃ | 3-F | IA-600 | 2-CF₃ | 3-Cl |
| IA-601 | 2-CF₃ | 3-Br | IA-602 | 2-CF₃ | 3-CH₃ |
| IA-603 | 2-CF₃ | 3-CH₂CH₃ | IA-604 | 2-CF₃ | 3-C(CH₃)₃ |
| IA-605 | 2-CF₃ | 3-OCH₃ | IA-606 | 2-CF₃ | 3-OCH₂CH₃ |
| IA-607 | 2-CF₃ | 3-CF₃ | IA-608 | 2-CF₃ | 3-CH(CF₃)₂ |
| IA-609 | 2-CF₃ | 3-OC(CH₃)₃ | IA-610 | 2-CF₃ | 3-CF(CF₃)₂ |
| IA-611 | 2-CF₃ | 3-OCF₃ | IA-612 | 2-CF₃ | 3-OCHF₂ |
| IA-613 | 2-CF₃ | 3-SCF₃ | IA-614 | 2-CF₃ | 3-SO₂CF₃ |
| IA-615 | 2-CF₃ | 3-CN | IA-616 | 2-CF₃ | 3-SCH₃ |
| IA-617 | 2-CF₃ | 3-SO₂CH₃ | IA-618 | 2-CF₃ | 3-OCF₂CF₃ |
| IA-619 | 2-CF₃ | 3- | IA-620 | 2-CF₃ | 3- |
| | | OCH₂CHCH₂ | | | OCH₂CH₂OCH₃ |
| IA-621 | 2-CF₃ | 3-OCH₂CF₃ | IA-622 | 2-CF₃ | 3-OCH₂CCH |
| IA-623 | 2-CF₃ | 3-OCH₂- | IA-624 | 2-CF₃ | 3-C(OH)(CF₃)₂ |
| | | cyclo-C₃H₅ | | | |
| IA-625 | 2-OCF₃ | 3-F | IA-626 | 2-OCF₃ | 3-Cl |
| IA-627 | 2-OCF₃ | 3-Br | IA-628 | 2-OCF₃ | 3-CH₃ |
| IA-629 | 2-OCF₃ | 3-CH₂CH₃ | IA-630 | 2-OCF₃ | 3-C(CH₃)₃ |
| IA-631 | 2-OCF₃ | 3-OCH₃ | IA-632 | 2-OCF₃ | 3-OCH₂CH₃ |
| IA-633 | 2-OCF₃ | 3-CF₃ | IA-634 | 2-OCF₃ | 3-CH(CF₃)₂ |
| IA-635 | 2-OCF₃ | 3-OC(CH₃)₃ | IA-636 | 2-OCF₃ | 3-CF(CF₃)₂ |
| IA-637 | 2-OCF₃ | 3-OCF₃ | IA-638 | 2-OCF₃ | 3-OCHF₂ |
| IA-639 | 2-OCF₃ | 3-SCF₃ | IA-640 | 2-OCF₃ | 3-SO₂CF₃ |
| IA-641 | 2-OCF₃ | 3-CN | IA-642 | 2-OCF₃ | 3-SCH₃ |
| IA-643 | 2-OCF₃ | 3-SO₂CH₃ | IA-644 | 2-OCF₃ | 3-OCF₂CF₃ |
| IA-645 | 2-OCF₃ | 3- | IA-646 | 2-OCF₃ | 3- |
| | | OCH₂CHCH₂ | | | OCH₂CH₂OCH₃ |
| IA-647 | 2-OCF₃ | 3-OCH₂CF₃ | IA-648 | 2-OCF₃ | 3-OCH₂CCH |
| IA-649 | 2-OCF₃ | 3-OCH₂- | IA-650 | 2-OCF₃ | 3-C(OH)(CF₃)₂ |
| | | cyclo-C₃H₅ | | | |
| IA-651 | 2-SCH₃ | 3-F | IA-652 | 2-SCH₃ | 3-Cl |
| IA-653 | 2-SCH₃ | 3-Br | IA-654 | 2-SCH₃ | 3-CH₃ |
| IA-655 | 2-SCH₃ | 3-CH₂CH₃ | IA-656 | 2-SCH₃ | 3-C(CH₃)₃ |
| IA-657 | 2-SCH₃ | 3-OCH₃ | IA-658 | 2-SCH₃ | 3-OCH₂CH₃ |
| IA-659 | 2-SCH₃ | 3-CF₃ | IA-660 | 2-SCH₃ | 3-CH(CF₃)₂ |
| IA-661 | 2-SCH₃ | 3-OC(CH₃)₃ | IA-662 | 2-SCH₃ | 3-CF(CF₃)₂ |
| IA-663 | 2-SCH₃ | 3-OCF₃ | IA-664 | 2-SCH₃ | 3-OCHF₂ |
| IA-665 | 2-SCH₃ | 3-SCF₃ | IA-666 | 2-SCH₃ | 3-SO₂CF₃ |
| IA-667 | 2-SCH₃ | 3-CN | IA-668 | 2-SCH₃ | 3-SCH₃ |
| IA-669 | 2-SCH₃ | 3-SO₂CH₃ | IA-670 | 2-SCH₃ | 3-OCF₂CF₃ |
| IA-671 | 2-SCH₃ | 3- | IA-672 | 2-SCH₃ | 3- |
| | | OCH₂CHCH₂ | | | OCH₂CH₂OCH₃ |
| IA-673 | 2-SCH₃ | 3-OCH₂CF₃ | IA-674 | 2-SCH₃ | 3-OCH₂CCH |
| IA-675 | 2-SCH₃ | 3-OCH₂- | IA-676 | 2-SCH₃ | 3-C(OH)(CF₃)₂ |
| | | cyclo-C₃H₅ | | | |
| IA-677 | 2-OCHF₂ | 3-F | IA-678 | 2-OCHF₂ | 3-Cl |
| IA-679 | 2-OCHF₂ | 3-Br | IA-680 | 2-OCHF₂ | 3-CH₃ |
| IA-681 | 2-OCHF₂ | 3-CH₂CH₃ | IA-682 | 2-OCHF₂ | 3-C(CH₃)₃ |
| IA-683 | 2-OCHF₂ | 3-OCH₃ | IA-684 | 2-OCHF₂ | 3-OCH₂CH₃ |
| IA-685 | 2-OCHF₂ | 3-CF₃ | IA-686 | 2-OCHF₂ | 3-CH(CF₃)₂ |
| IA-687 | 2-OCHF₂ | 3-OC(CH₃)₃ | IA-688 | 2-OCHF₂ | 3-CF(CF₃)₂ |
| IA-689 | 2-OCHF₂ | 3-OCF₃ | IA-690 | 2-OCHF₂ | 3-OCHF₂ |
| IA-691 | 2-OCHF₂ | 3-SCF₃ | IA-692 | 2-OCHF₂ | 3-SO₂CF₃ |
| IA-693 | 2-OCHF₂ | 3-CN | IA-694 | 2-OCHF₂ | 3-SCH₃ |
| IA-695 | 2-OCHF₂ | 3-SO₂CH₃ | IA-696 | 2-OCHF₂ | 3-OCF₂CF₃ |
| IA-697 | 2-OCHF₂ | 3- | IA-698 | 2-OCHF₂ | 3- |
| | | OCH₂CHCH₂ | | | OCH₂CH₂OCH₃ |
| IA-699 | 2-OCHF₂ | 3-OCH₂CF₃ | IA-700 | 2-OCHF₂ | 3-OCH₂CCH |
| IA-701 | 2-OCHF₂ | 3-OCH₂- | IA-702 | 2-OCHF₂ | 3-C(OH)(CF₃)₂ |
| | | cyclo-C₃H₅ | | | |
| IA-703 | 2-F | 5-F | IA-704 | 2-F | 5-Cl |
| IA-705 | 2-F | 5-Br | IA-706 | 2-F | 5-CH₃ |
| IA-707 | 2-F | 5-CH₂CH₃ | IA-708 | 2-F | 5-C(CH₃)₃ |
| IA-709 | 2-F | 5-OCH₃ | IA-710 | 2-F | 5-OCH₂CH₃ |
| IA-711 | 2-F | 5-CF₃ | IA-712 | 2-F | 5-CH(CF₃)₂ |
| IA-713 | 2-F | 5-OC(CH₃)₃ | IA-714 | 2-F | 5-CF(CF₃)₂ |
| IA-715 | 2-F | 5-OCF₃ | IA-716 | 2-F | 5-OCHF₂ |
| IA-717 | 2-F | 5-SCF₃ | IA-718 | 2-F | 5-SO₂CF₃ |
| IA-719 | 2-F | 5-CN | IA-720 | 2-F | 5-SCH₃ |
| IA-721 | 2-F | 5-SO₂CH₃ | IA-722 | 2-F | 5-OCF₂CF₃ |
| IA-723 | 2-F | 5- | IA-724 | 2-F | 5- |
| | | OCH₂CHCH₂ | | | OCH₂CH₂OCH₃ |
| IA-725 | 2-F | 5-OCH₂CF₃ | IA-726 | 2-F | 5-OCH₂CCH |
| IA-727 | 2-F | 5-OCH₂- | IA-728 | 2-F | 5-C(OH)(CF₃)₂ |
| | | cyclo-C₃H₅ | | | |
| IA-729 | 2-Cl | 5-F | IA-730 | 2-Cl | 5-Cl |
| IA-731 | 2-Cl | 5-Br | IA-732 | 2-Cl | 5-CH₃ |
| IA-733 | 2-Cl | 5-CH₂CH₃ | IA-734 | 2-Cl | 5-C(CH₃)₃ |
| IA-735 | 2-Cl | 5-OCH₃ | IA-736 | 2-Cl | 5-OCH₂CH₃ |
| IA-737 | 2-Cl | 5-CF₃ | IA-738 | 2-Cl | 5-CH(CF₃)₂ |
| IA-739 | 2-Cl | 5-OC(CH₃)₃ | IA-740 | 2-Cl | 5-CF(CF₃)₂ |
| IA-741 | 2-Cl | 5-OCF₃ | IA-742 | 2-Cl | 5-OCHF₂ |
| IA-743 | 2-Cl | 5-SCF₃ | IA-744 | 2-Cl | 5-SO₂CF₃ |
| IA-745 | 2-Cl | 5-CN | IA-746 | 2-Cl | 5-SCH₃ |
| IA-747 | 2-Cl | 5-SO₂CH₃ | IA-748 | 2-Cl | 5-OCF₂CF₃ |
| IA-749 | 2-Cl | 5- | IA-750 | 2-Cl | 5- |
| | | OCH₂CHCH₂ | | | OCH₂CH₂OCH₃ |
| IA-751 | 2-Cl | 5-OCH₂CF₃ | IA-752 | 2-Cl | 5-OCH₂CCH |
| IA-753 | 2-Cl | 5-OCH₂- | IA-754 | 2-Cl | 5-C(OH)(CF₃)₂ |
| | | cyclo-C₃H₅ | | | |
| IA-755 | 2-Br | 5-F | IA-756 | 2-Br | 5-Cl |
| IA-757 | 2-Br | 5-Br | IA-758 | 2-Cl | 5-CH₃ |
| IA-759 | 2-Br | 5-CH₂CH₃ | IA-760 | 2-Br | 5-C(CH₃)₃ |
| IA-761 | 2-Br | 5-OCH₃ | IA-762 | 2-Br | 5-OCH₂CH₃ |
| IA-763 | 2-Br | 5-CF₃ | IA-764 | 2-Br | 5-CH(CF₃)₂ |
| IA-765 | 2-Br | 5-OC(CH₃)₃ | IA-766 | 2-Br | 5-CF(CF₃)₂ |
| IA-767 | 2-Br | 5-OCF₃ | IA-768 | 2-Br | 5-OCHF₂ |
| IA-769 | 2-Br | 5-SCF₃ | IA-770 | 2-Br | 5-SO₂CF₃ |
| IA-771 | 2-Br | 5-CN | IA-772 | 2-Br | 5-SCH₃ |
| IA-773 | 2-Br | 5-SO₂CH₃ | IA-774 | 2-Br | 5-OCF₂CF₃ |
| IA-775 | 2-Br | 5- | IA-776 | 2-Br | 5- |
| | | OCH₂CHCH₂ | | | OCH₂CH₂OCH₃ |
| IA-777 | 2-Br | 5-OCH₂CF₃ | IA-778 | 2-Br | 5-OCH₂CCH |
| IA-779 | 2-Br | 5-OCH₂- | IA-780 | 2-Br | 5-C(OH)(CF₃)₂ |
| | | cyclo-C₃H₅ | | | |
| IA-781 | 2-CH₃ | 5-F | IA-782 | 2-CH₃ | 5-Cl |
| IA-783 | 2-CH₃ | 5-Br | IA-784 | 2-CH₃ | 5-CH₃ |
| IA-785 | 2-CH₃ | 5-CH₂CH₃ | IA-786 | 2-CH₃ | 5-C(CH₃)₃ |
| IA-787 | 2-CH₃ | 5-OCH₃ | IA-788 | 2-CH₃ | 5-OCH₂CH₃ |
| IA-789 | 2-CH₃ | 5-CF₃ | IA-790 | 2-CH₃ | 5-CH(CF₃)₂ |
| IA-791 | 2-CH₃ | 5-OC(CH₃)₃ | IA-792 | 2-CH₃ | 5-CF(CF₃)₂ |
| IA-793 | 2-CH₃ | 5-OCF₃ | IA-794 | 2-CH₃ | 5-OCHF₂ |
| IA-795 | 2-CH₃ | 5-SCF₃ | IA-796 | 2-CH₃ | 5-SO₂CF₃ |
| IA-797 | 2-CH₃ | 5-CN | IA-798 | 2-CH₃ | 5-SCH₃ |
| IA-799 | 2-CH₃ | 5-SO₂CH₃ | IA-800 | 2-CH₃ | 5-OCF₂CF₃ |
| IA-801 | 2-CH₃ | 5- | IA-802 | 2-CH₃ | 5- |
| | | OCH₂CHCH₂ | | | OCH₂CH₂OCH₃ |
| IA-803 | 2-CH₃ | 5-OCH₂CF₃ | IA-804 | 2-CH₃ | 5-OCH₂CCH |
| IA-805 | 2-CH₃ | 5-OCH₂- | IA-806 | 2-CH₃ | 5-C(OH)(CF₃)₂ |
| | | cyclo-C₃H₅ | | | |
| IA-807 | 2-OCH₃ | 5-F | IA-808 | 2-OCH₃ | 5-Cl |
| IA-809 | 2-OCH₃ | 5-Br | IA-810 | 2-OCH₃ | 5-CH₃ |
| IA-811 | 2-OCH₃ | 5-CH₂CH₃ | IA-812 | 2-OCH₃ | 5-C(CH₃)₃ |
| IA-813 | 2-OCH₃ | 5-OCH₃ | IA-814 | 2-OCH₃ | 5-OCH₂CH₃ |
| IA-815 | 2-OCH₃ | 5-CF₃ | IA-816 | 2-OCH₃ | 5-CH(CF₃)₂ |
| IA-817 | 2-OCH₃ | 5-OC(CH₃)₃ | IA-818 | 2-OCH₃ | 5-CF(CF₃)₂ |
| IA-819 | 2-OCH₃ | 5-OCF₃ | IA-820 | 2-OCH₃ | 5-OCHF₂ |
| IA-821 | 2-OCH₃ | 5-SCF₃ | IA-822 | 2-OCH₃ | 5-SO₂CF₃ |
| IA-823 | 2-OCH₃ | 5-CN | IA-824 | 2-OCH₃ | 5-SCH₃ |
| IA-825 | 2-OCH₃ | 5-SO₂CH₃ | IA-826 | 2-OCH₃ | 5-OCF₂CF₃ |
| IA-827 | 2-OCH₃ | 5- | IA-828 | 2-OCH₃ | 5- |
| | | OCH₂CHCH₂ | | | OCH₂CH₂OCH₃ |
| IA-829 | 2-OCH₃ | 5-OCH₂CF₃ | IA-830 | 2-OCH₃ | 5-OCH₂CCH |
| IA-831 | 2-OCH₃ | 5-OCH₂- | IA-832 | 2-OCH₃ | 5-C(OH)(CF₃)₂ |
| | | cyclo-C₃H₅ | | | |
| IA-833 | 2-CF₃ | 5-F | IA-834 | 2-CF₃ | 5-Cl |
| IA-835 | 2-CF₃ | 5-Br | IA-836 | 2-CF₃ | 5-CH₃ |
| IA-837 | 2-CF₃ | 5-CH₂CH₃ | IA-838 | 2-CF₃ | 5-C(CH₃)₃ |
| IA-839 | 2-CF₃ | 5-OCH₃ | IA-840 | 2-CF₃ | 5-OCH₂CH₃ |
| IA-841 | 2-CF₃ | 5-CF₃ | IA-842 | 2-CF₃ | 5-CH(CF₃)₂ |
| IA-843 | 2-CF₃ | 5-OC(CH₃)₃ | IA-844 | 2-CF₃ | 5-CF(CF₃)₂ |
| IA-845 | 2-CF₃ | 5-OCF₃ | IA-846 | 2-CF₃ | 5-OCHF₂ |
| IA-847 | 2-CF₃ | 5-SCF₃ | IA-848 | 2-CF₃ | 5-SO₂CF₃ |
| IA-849 | 2-CF₃ | 5-CN | IA-850 | 2-CF₃ | 5-SCH₃ |
| IA-851 | 2-CF₃ | 5-SO₂CH₃ | IA-852 | 2-CF₃ | 5-OCF₂CF₃ |
| IA-853 | 2-CF₃ | 5- | IA-854 | 2-CF₃ | 5- |
| | | OCH₂CHCH₂ | | | OCH₂CH₂OCH₃ |
| IA-855 | 2-CF₃ | 5-OCH₂CF₃ | IA-856 | 2-CF₃ | 5-OCH₂CCH |
| IA-857 | 2-CF₃ | 5-OCH₂- | IA-858 | 2-CF₃ | 5-C(OH)(CF₃)₂ |
| | | cyclo-C₃H₅ | | | |
| IA-859 | 2-OCF₃ | 5-F | IA-860 | 2-OCF₃ | 5-Cl |
| IA-861 | 2-OCF₃ | 5-Br | IA-862 | 2-OCF₃ | 5-CH₃ |
| IA-863 | 2-OCF₃ | 5-CH₂CH₃ | IA-864 | 2-OCF₃ | 5-C(CH₃)₃ |
| IA-865 | 2-OCF₃ | 5-OCH₃ | IA-866 | 2-OCF₃ | 5-OCH₂CH₃ |
| IA-867 | 2-OCF₃ | 5-CF₃ | IA-868 | 2-OCF₃ | 5-CH(CF₃)₂ |
| IA-869 | 2-OCF₃ | 5-OC(CH₃)₃ | IA-870 | 2-OCF₃ | 5-CF(CF₃)₂ |
| IA-871 | 2-OCF₃ | 5-OCF₃ | IA-872 | 2-OCF₃ | 5-OCHF₂ |
| IA-873 | 2-OCF₃ | 5-SCF₃ | IA-874 | 2-OCF₃ | 5-SO₂CF₃ |
| IA-875 | 2-OCF₃ | 5-CN | IA-876 | 2-OCF₃ | 5-SCH₃ |
| IA-877 | 2-OCF₃ | 5-SO₂CH₃ | IA-878 | 2-OCF₃ | 5-OCF₂CF₃ |
| IA-879 | 2-OCF₃ | 5- | IA-880 | 2-OCF₃ | 5- |
| | | OCH₂CHCH₂ | | | OCH₂CH₂OCH₃ |
| IA-881 | 2-OCF₃ | 5-OCH₂CF₃ | IA-882 | 2-OCF₃ | 5-OCH₂CCH |
| IA-883 | 2-OCF₃ | 5-OCH₂- | IA-884 | 2-OCF₃ | 5-C(OH)(CF₃)₂ |
| | | cyclo-C₃H₅ | | | |
| IA-885 | 2-SCH₃ | 5-F | IA-886 | 2-SCH₃ | 5-Cl |
| IA-887 | 2-SCH₃ | 5-Br | IA-888 | 2-SCH₃ | 5-CH₃ |
| IA-889 | 2-SCH₃ | 5-CH₂CH₃ | IA-890 | 2-SCH₃ | 5-C(CH₃)₃ |
| IA-891 | 2-SCH₃ | 5-OCH₃ | IA-892 | 2-SCH₃ | 5-OCH₂CH₃ |
| IA-893 | 2-SCH₃ | 5-CF₃ | IA-894 | 2-SCH₃ | 5-CH(CF₃)₂ |
| IA-895 | 2-SCH₃ | 5-OC(CH₃)₃ | IA-896 | 2-SCH₃ | 5-CF(CF₃)₂ |
| IA-897 | 2-SCH₃ | 5-OCF₃ | IA-898 | 2-SCH₃ | 5-OCHF₂ |
| IA-899 | 2-SCH₃ | 5-SCF₃ | IA-900 | 2-SCH₃ | 5-SO₂CF₃ |
| IA-901 | 2-SCH₃ | 5-CN | IA-902 | 2-SCH₃ | 5-SCH₃ |
| IA-903 | 2-SCH₃ | 5-SO₂CH₃ | IA-904 | 2-SCH₃ | 5-OCF₂CF₃ |
| IA-905 | 2-SCH₃ | 5- | IA-906 | 2-SCH₃ | 5- |
| | | OCH₂CHCH₂ | | | OCH₂CH₂OCH₃ |
| IA-907 | 2-SCH₃ | 5-OCH₂CF₃ | IA-908 | 2-SCH₃ | 5-OCH₂CCH |
| IA-909 | 2-SCH₃ | 5-OCH₂- | IA-910 | 2-SCH₃ | 5-C(OH)(CF₃)₂ |
| | | cyclo-C₃H₅ | | | |
| IA-911 | 2-OCHF₂ | 5-F | IA-912 | 2-OCHF₂ | 5-Cl |
| IA-913 | 2-OCHF₂ | 5-Br | IA-914 | 2-OCHF₂ | 5-CH₃ |
| IA-915 | 2-OCHF₂ | 5-CH₂CH₃ | IA-916 | 2-OCHF₂ | 5-C(CH₃)₃ |
| IA-917 | 2-OCHF₂ | 5-OCH₃ | IA-918 | 2-OCHF₂ | 5-OCH₂CH₃ |
| IA-919 | 2-OCHF₂ | 5-CF₃ | IA-920 | 2-OCHF₂ | 5-CH(CF₃)₂ |
| IA-921 | 2-OCHF₂ | 5-OC(CH₃)₃ | IA-922 | 2-OCHF₂ | 5-CF(CF₃)₂ |
| IA-923 | 2-OCHF₂ | 5-OCF₃ | IA-924 | 2-OCHF₂ | 5-OCHF₂ |
| IA-925 | 2-OCHF₂ | 5-SCF₃ | IA-926 | 2-OCHF₂ | 5-SO₂CF₃ |
| IA-927 | 2-OCHF₂ | 5-CN | IA-928 | 2-OCHF₂ | 5-SCH₃ |
| IA-929 | 2-OCHF₂ | 5-SO₂CH₃ | IA-930 | 2-OCHF₂ | 5-OCF₂CF₃ |
| IA-931 | 2-OCHF₂ | 5- | IA-932 | 2-OCHF₂ | 5- |
| | | OCH₂CHCH₂ | | | OCH₂CH₂OCH₃ |
| IA-933 | 2-OCHF₂ | 5-OCH₂CF₃ | IA-934 | 2-OCHF₂ | 5-OCH₂CCH |
| IA-935 | 2-OCHF₂ | 5-OCH₂- | IA-936 | 2-OCHF₂ | 5-C(OH)(CF₃)₂ |
| | | cyclo-C₃H₅ | | | |
| IA-937 | 2-CH₂CH₃ | 5-F | IA-938 | 2-CH₂CH₃ | 5-Cl |
| IA-939 | 2-CH₂CH₃ | 5-Br | IA-940 | 2-CH₂CH₃ | 5-CH₃ |
| IA-941 | 2-CH₂CH₃ | 5-CH₂CH₃ | IA-942 | 2-CH₂CH₃ | 5-C(CH₃)₃ |
| IA-943 | 2-CH₂CH₃ | 5-OCH₃ | IA-944 | 2-CH₂CH₃ | 5-OCH₂CH₃ |
| IA-945 | 2-CH₂CH₃ | 5-CF₃ | IA-946 | 2-CH₂CH₃ | 5-CH(CF₃)₂ |
| IA-947 | 2-CH₂CH₃ | 5-OC(CH₃)₃ | IA-948 | 2-CH₂CH₃ | 5-CF(CF₃)₂ |
| IA-949 | 2-CH₂CH₃ | 5-OCF₃ | IA-950 | 2-CH₂CH₃ | 5-OCHF₂ |
| IA-951 | 2-CH₂CH₃ | 5-SCF₃ | IA-952 | 2-CH₂CH₃ | 5-SO₂CF₃ |
| IA-953 | 2-CH₂CH₃ | 5-CN | IA-954 | 2-CH₂CH₃ | 5-SCH₃ |
| IA-955 | 2-CH₂CH₃ | 5-SO₂CH₃ | IA-956 | 2-CH₂CH₃ | 5-OCF₂CF₃ |
| IA-957 | 2-CH₂CH₃ | 5- | IA-958 | 2-CH₂CH₃ | 5- |
| | | OCH₂CHCH₂ | | | OCH₂CH₂OCH₃ |
| IA-959 | 2-CH₂CH₃ | 5-OCH₂CF₃ | IA-960 | 2-CH₂CH₃ | 5-OCH₂CCH |
| IA-961 | 2-CH₂CH₃ | 5-OCH₂- | IA-962 | 2-CH₂CH₃ | 5-C(OH)(CF₃)₂ |
| | | cyclo-C₃H₅ | | | |
| IA-963 | 2-C(CH₃)₃ | 5-F | IA-964 | 2-C(CH₃)₃ | 5-Cl |
| IA-965 | 2-C(CH₃)₃ | 5-Br | IA-966 | 2-C(CH₃)₃ | 5-CH₃ |
| IA-967 | 2-C(CH₃)₃ | 5-CH₂CH₃ | IA-968 | 2-C(CH₃)₃ | 5-C(CH₃)₃ |
| IA-969 | 2-C(CH₃)₃ | 5-OCH₃ | IA-970 | 2-C(CH₃)₃ | 5-OCH₂CH₃ |
| IA-971 | 2-C(CH₃)₃ | 5-CF₃ | IA-972 | 2-C(CH₃)₃ | 5-CH(CF₃)₂ |
| IA-973 | 2-C(CH₃)₃ | 5-OC(CH₃)₃ | IA-974 | 2-C(CH₃)₃ | 5-CF(CF₃)₂ |
| IA-975 | 2-C(CH₃)₃ | 5-OCF₃ | IA-976 | 2-C(CH₃)₃ | 5-OCHF₂ |
| IA-977 | 2-C(CH₃)₃ | 5-SCF₃ | IA-978 | 2-C(CH₃)₃ | 5-SO₂CF₃ |
| IA-979 | 2-C(CH₃)₃ | 5-CN | IA-980 | 2-C(CH₃)₃ | 5-SCH₃ |
| IA-981 | 2-C(CH₃)₃ | 5-SO₂CH₃ | IA-982 | 2-C(CH₃)₃ | 5-OCF₂CF₃ |
| IA-983 | 2-C(CH₃)₃ | 5- | IA-984 | 2-C(CH₃)₃ | 5- |
| | | OCH₂CHCH₂ | | | OCH₂CH₂OCH₃ |
| IA-985 | 2-C(CH₃)₃ | 5-OCH₂CF₃ | IA-986 | 2-C(CH₃)₃ | 5-OCH₂CCH |
| IA-987 | 2-C(CH₃)₃ | 5-OCH₂- | IA-988 | 2-C(CH₃)₃ | 5-C(OH)(CF₃)₂ |
| | | cyclo-C₃H₅ | | | |
| IA-989 | 2-OCH₂CH₃ | 5-F | IA-990 | 2-OCH₂CH₃ | 5-Cl |
| IA-991 | 2-OCH₂CH₃ | 5-Br | IA-992 | 2-OCH₂CH₃ | 5-CH₃ |
| IA-993 | 2-OCH₂CH₃ | 5-CH₂CH₃ | IA-994 | 2-OCH₂CH₃ | 5-C(CH₃)₃ |
| IA-995 | 2-OCH₂CH₃ | 5-OCH₃ | IA-996 | 2-OCH₂CH₃ | 5-OCH₂CH₃ |
| IA-997 | 2-OCH₂CH₃ | 5-CF₃ | IA-998 | 2-OCH₂CH₃ | 5-CH(CF₃)₂ |
| IA-999 | 2-OCH₂CH₃ | 5-OC(CH₃)₃ | IA-1000 | 2-OCH₂CH₃ | 5-CF(CF₃)₂ |
| IA-1001 | 2-OCH₂CH₃ | 5-OCF₃ | IA-1002 | 2-OCH₂CH₃ | 5-OCHF₂ |
| IA-1003 | 2-OCH₂CH₃ | 5-SCF₃ | IA-1004 | 2-OCH₂CH₃ | 5-SO₂CF₃ |
| IA-1005 | 2-OCH₂CH₃ | 5-CN | IA-1006 | 2-OCH₂CH₃ | 5-SCH₃ |
| IA-1007 | 2-OCH₂CH₃ | 5-SO₂CH₃ | IA-1008 | 2-OCH₂CH₃ | 5-OCF₂CF₃ |
| IA-1009 | 2-OCH₂CH₃ | 5- | IA-1010 | 2-OCH₂CH₃ | 5- |
| | | OCH₂CHCH₂ | | | OCH₂CH₂OCH₃ |
| IA-1011 | 2-OCH₂CH₃ | 5-OCH₂CF₃ | IA-1012 | 2-OCH₂CH₃ | 5-OCH₂CCH |
| IA-1013 | 2-OCH₂CH₃ | 5-OCH₂- | IA-1014 | 2-OCH₂CH₃ | 5-C(OH)(CF₃)₂ |
| | | cyclo-C₃H₅ | | | |
| IA-1015 | 2-CH(CF₃)₂ | 5-F | IA-1016 | 2-CH(CF₃)₂ | 5-Cl |
| IA-1017 | 2-CH(CF₃)₂ | 5-Br | IA-1018 | 2-CH(CF₃)₂ | 5-CH₃ |
| IA-1019 | 2-CH(CF₃)₂ | 5-CH₂CH₃ | IA-1020 | 2-CH(CF₃)₂ | 5-C(CH₃)₃ |
| IA-1021 | 2-CH(CF₃)₂ | 5-OCH₃ | IA-1022 | 2-CH(CF₃)₂ | 5-OCH₂CH₃ |
| IA-1023 | 2-CH(CF₃)₂ | 5-CF₃ | IA-1024 | 2-CH(CF₃)₂ | 5-CH(CF₃)₂ |
| IA-1025 | 2-CH(CF₃)₂ | 5-OC(CH₃)₃ | IA-1026 | 2-CH(CF₃)₂ | 5-CF(CF₃)₂ |
| IA-1027 | 2-CH(CF₃)₂ | 5-OCF₃ | IA-1028 | 2-CH(CF₃)₂ | 5-OCHF₂ |
| IA-1029 | 2-CH(CF₃)₂ | 5-SCF₃ | IA-1030 | 2-CH(CF₃)₂ | 5-SO₂CF₃ |
| IA-1031 | 2-CH(CF₃)₂ | 5-CN | IA-1032 | 2-CH(CF₃)₂ | 5-SCH₃ |
| IA-1033 | 2-CH(CF₃)₂ | 5-SO₂CH₃ | IA-1034 | 2-CH(CF₃)₂ | 5-OCF₂CF₃ |
| IA-1035 | 2-CH(CF₃)₂ | 5- | IA-1036 | 2-CH(CF₃)₂ | 5- |
| | | OCH₂CHCH₂ | | | OCH₂CH₂OCH₃ |
| IA-1037 | 2-CH(CF₃)₂ | 5-OCH₂CF₃ | IA-1038 | 2-CH(CF₃)₂ | 5-OCH₂CCH |
| IA-1039 | 2-CH(CF₃)₂ | 5-OCH₂- | IA-1040 | 2-CH(CF₃)₂ | 5-C(OH)(CF₃)₂ |
| | | cyclo-C₃H₅ | | | |
| IA-1041 | 2- | 5-F | IA-1042 | 2- | 5-Cl |
| | C(OH)(CF₃)₂ | | | C(OH)(CF₃)₂ | |
| IA-1043 | 2- | 5-Br | IA-1044 | 2- | 5-CH₃ |
| | C(OH)(CF₃)₂ | | | C(OH)(CF₃)₂ | |
| IA-1045 | 2- | 5-CH₂CH₃ | IA-1046 | 2- | 5-C(CH₃)₃ |
| | C(OH)(CF₃)₂ | | | C(OH)(CF₃)₂ | |
| IA-1047 | 2- | 5-OCH₃ | IA-1048 | 2- | 5-OCH₂CH₃ |
| | C(OH)(CF₃)₂ | | | C(OH)(CF₃)₂ | |
| IA-1049 | 2- | 5-CF₃ | IA-1050 | 2- | 5-CH(CF₃)₂ |
| | C(OH)(CF₃)₂ | | | C(OH)(CF₃)₂ | |
| IA-1051 | 2- | 5-OC(CH₃)₃ | IA-1052 | 2- | 5-CF(CF₃)₂ |
| | C(OH)(CF₃)₂ | | | C(OH)(CF₃)₂ | |
| IA-1053 | 2- | 5-OCF₃ | IA-1054 | 2- | 5-OCHF₂ |
| | C(OH)(CF₃)₂ | | | C(OH)(CF₃)₂ | |
| IA-1055 | 2- | 5-SCF₃ | IA-1056 | 2- | 5-SO₂CF₃ |
| | C(OH)(CF₃)₂ | | | C(OH)(CF₃)₂ | |
| IA-1057 | 2- | 5-CN | IA-1058 | 2- | 5-SCH₃ |
| | C(OH)(CF₃)₂ | | | C(OH)(CF₃)₂ | |
| IA-1059 | 2- | 5-SO₂CH₃ | IA-1060 | 2- | 5-OCF₂CF₃ |
| | C(OH)(CF₃)₂ | | | C(OH)(CF₃)₂ | |
| IA-1061 | 2- | 5- | IA-1062 | 2- | 5- |
| | C(OH)(CF₃)₂ | OCH₂CHCH₂ | | C(OH)(CF₃)₂ | OCH₂CH₂OCH₃ |
| IA-1063 | 2- | 5-OCH₂CF₃ | IA-1064 | 2- | 5-OCH₂CCH |
| | C(OH)(CF₃)₂ | | | C(OH)(CF₃)₂ | |
| IA-1065 | 2- | 5-OCH₂- | IA-1066 | 2- | 5-C(OH)(CF₃)₂ |
| | C(OH)(CF₃)₂ | cyclo-C₃H₅ | | C(OH)(CF₃)₂ | |
| IA-1067 | 2-CF(CF₃)₂ | 5-F | IA-1068 | 2-CF(CF₃)₂ | 5-Cl |
| IA-1069 | 2-CF(CF₃)₂ | 5-Br | IA-1070 | 2-CF(CF₃)₂ | 5-CH₃ |
| IA-1071 | 2-CF(CF₃)₂ | 5-CH₂CH₃ | IA-1072 | 2-CF(CF₃)₂ | 5-C(CH₃)₃ |
| IA-1073 | 2-CF(CF₃)₂ | 5-OCH₃ | IA-1074 | 2-CF(CF₃)₂ | 5-OCH₂CH₃ |
| IA-1075 | 2-CF(CF₃)₂ | 5-CF₃ | IA-1076 | 2-CF(CF₃)₂ | 5-CH(CF₃)₂ |
| IA-1077 | 2-CF(CF₃)₂ | 5-OC(CH₃)₃ | IA-1078 | 2-CF(CF₃)₂ | 5-CF(CF₃)₂ |
| IA-1079 | 2-CF(CF₃)₂ | 5-OCF₃ | IA-1080 | 2-CF(CF₃)₂ | 5-OCHF₂ |
| IA-1081 | 2-CF(CF₃)₂ | 5-SCF₃ | IA-1082 | 2-CF(CF₃)₂ | 5-SO₂CF₃ |
| IA-1083 | 2-CF(CF₃)₂ | 5-CN | IA-1084 | 2-CF(CF₃)₂ | 5-SCH₃ |
| IA-1085 | 2-CF(CF₃)₂ | 5-SO₂CH₃ | IA-1086 | 2-CF(CF₃)₂ | 5-OCF₂CF₃ |
| IA-1087 | 2-CF(CF₃)₂ | 5- | IA-1088 | 2-CF(CF₃)₂ | 5- |
| | | OCH₂CHCH₂ | | | OCH₂CH₂OCH₃ |
| IA-1089 | 2-CF(CF₃)₂ | 5-OCH₂CF₃ | IA-1090 | 2-CF(CF₃)₂ | 5-OCH₂CCH |
| IA-1091 | 2-CF(CF₃)₂ | 5-OCH₂- | IA-1092 | 2-CF(CF₃)₂ | 5-C(OH)(CF₃)₂ |
| | | cyclo-C₃H₅ | | | |
| IA-1093 | 2-SCF₃ | 5-F | IA-1094 | 2-SCF₃ | 5-Cl |
| IA-1095 | 2-SCF₃ | 5-Br | IA-1096 | 2-SCF₃ | 5-CH₃ |
| IA-1097 | 2-SCF₃ | 5-CH₂CH₃ | IA-1098 | 2-SCF₃ | 5-C(CH₃)₃ |
| IA-1099 | 2-SCF₃ | 5-OCH₃ | IA-1100 | 2-SCF₃ | 5-OCH₂CH₃ |
| IA-1101 | 2-SCF₃ | 5-CF₃ | IA-1102 | 2-SCF₃ | 5-CH(CF₃)₂ |
| IA-1103 | 2-SCF₃ | 5-OC(CH₃)₃ | IA-1104 | 2-SCF₃ | 5-CF(CF₃)₂ |
| IA-1105 | 2-SCF₃ | 5-OCF₃ | IA-1106 | 2-SCF₃ | 5-OCHF₂ |
| IA-1107 | 2-SCF₃ | 5-SCF₃ | IA-1108 | 2-SCF₃ | 5-SO₂CF₃ |
| IA-1109 | 2-SCF₃ | 5-CN | IA-1110 | 2-SCF₃ | 5-SCH₃ |
| IA-1111 | 2-SCF₃ | 5-SO₂CH₃ | IA-1112 | 2-SCF₃ | 5-OCF₂CF₃ |
| IA-1113 | 2-SCF₃ | 5- | IA-1114 | 2-SCF₃ | 5- |
| | | OCH₂CHCH₂ | | | OCH₂CH₂OCH₃ |
| IA-1115 | 2-SCF₃ | 5-OCH₂CF₃ | IA-1116 | 2-SCF₃ | 5-OCH₂CCH |
| IA-1117 | 2-SCF₃ | 5-OCH₂- | IA-1118 | 2-SCF₃ | 5-C(OH)(CF₃)₂ |
| | | cyclo-C₃H₅ | | | |
| IA-1119 | 2-SO₂CF₃ | 5-F | IA-1120 | 2-SO₂CF₃ | 5-Cl |
| IA-1121 | 2-SO₂CF₃ | 5-Br | IA-1122 | 2-SO₂CF₃ | 5-CH₃ |
| IA-1123 | 2-SO₂CF₃ | 5-CH₂CH₃ | IA-1124 | 2-SO₂CF₃ | 5-C(CH₃)₃ |
| IA-1125 | 2-SO₂CF₃ | 5-OCH₃ | IA-1126 | 2-SO₂CF₃ | 5-OCH₂CH₃ |
| IA-1127 | 2-SO₂CF₃ | 5-CF₃ | IA-1128 | 2-SO₂CF₃ | 5-CH(CF₃)₂ |
| IA-1129 | 2-SO₂CF₃ | 5-OC(CH₃)₃ | IA-1130 | 2-SO₂CF₃ | 5-CF(CF₃)₂ |
| IA-1131 | 2-SO₂CF₃ | 5-OCF₃ | IA-1132 | 2-SO₂CF₃ | 5-OCHF₂ |
| IA-1133 | 2-SO₂CF₃ | 5-SCF₃ | IA-1134 | 2-SO₂CF₃ | 5-SO₂CF₃ |
| IA-1135 | 2-SO₂CF₃ | 5-CN | IA-1136 | 2-SO₂CF₃ | 5-SCH₃ |
| IA-1137 | 2-SO₂CF₃ | 5-SO₂CH₃ | IA-1138 | 2-SO₂CF₃ | 5-OCF₂CF₃ |
| IA-1139 | 2-SO₂CF₃ | 5- | IA-1140 | 2-SO₂CF₃ | 5- |
| | | OCH₂CHCH₂ | | | OCH₂CH₂OCH₃ |
| IA-1141 | 2-SO₂CF₃ | 5-OCH₂CF₃ | IA-1142 | 2-SO₂CF₃ | 5-OCH₂CCH |
| IA-1143 | 2-SO₂CF₃ | 5-OCH₂- | IA-1144 | 2-SO₂CF₃ | 5-C(OH)(CF₃)₂ |
| | | cyclo-C₃H₅ | | | |
| IA-1145 | 2-SO₂CH₃ | 5-F | IA-1146 | 2-SO₂CH₃ | 5-Cl |
| IA-1147 | 2-SO₂CH₃ | 5-Br | IA-1148 | 2-SO₂CH₃ | 5-CH₃ |
| IA-1149 | 2-SO₂CH₃ | 5-CH₂CH₃ | IA-1150 | 2-SO₂CH₃ | 5-C(CH₃)₃ |
| IA-1151 | 2-SO₂CH₃ | 5-OCH₃ | IA-1152 | 2-SO₂CH₃ | 5-OCH₂CH₃ |
| IA-1153 | 2-SO₂CH₃ | 5-CF₃ | IA-1154 | 2-SO₂CH₃ | 5-CH(CF₃)₂ |
| IA-1155 | 2-SO₂CH₃ | 5-OC(CH₃)₃ | IA-1156 | 2-SO₂CH₃ | 5-CF(CF₃)₂ |
| IA-1157 | 2-SO₂CH₃ | 5-OCF₃ | IA-1158 | 2-SO₂CH₃ | 5-OCHF₂ |
| IA-1159 | 2-SO₂CH₃ | 5-SCF₃ | IA-1160 | 2-SO₂CH₃ | 5-SO₂CF₃ |
| IA-1161 | 2-SO₂CH₃ | 5-CN | IA-1162 | 2-SO₂CH₃ | 5-SCH₃ |
| IA-1163 | 2-SO₂CH₃ | 5-SO₂CH₃ | IA-1164 | 2-SO₂CH₃ | 5-OCF₂CF₃ |
| IA-1165 | 2-SO₂CH₃ | 5- | IA-1166 | 2-SO₂CH₃ | 5- |
| | | OCH₂CHCH₂ | | | OCH₂CH₂OCH₃ |
| IA-1167 | 2-SO₂CH₃ | 5-OCH₂CF₃ | IA-1168 | 2-SO₂CH₃ | 5-OCH₂CCH |
| IA-1169 | 2-SO₂CH₃ | 5-OCH₂- | IA-1170 | 2-SO₂CH₃ | 5-C(OH)(CF₃)₂ |
| | | cyclo-C₃H₅ | | | |
| IA-1171 | 2-CN | 5-F | IA-1172 | 2-CN | 5-Cl |
| IA-1173 | 2-CN | 5-Br | IA-1174 | 2-CN | 5-CH₃ |
| IA-1175 | 2-CN | 5-CH₂CH₃ | IA-1176 | 2-CN | 5-C(CH₃)₃ |
| IA-1177 | 2-CN | 5-OCH₃ | IA-1178 | 2-CN | 5-OCH₂CH₃ |
| IA-1179 | 2-CN | 5-CF₃ | IA-1180 | 2-CN | 5-CH(CF₃)₂ |
| IA-1181 | 2-CN | 5-OC(CH₃)₃ | IA-1182 | 2-CN | 5-CF(CF₃)₂ |
| IA-1183 | 2-CN | 5-OCF₃ | IA-1184 | 2-CN | 5-OCHF₂ |
| IA-1185 | 2-CN | 5-SCF₃ | IA-1186 | 2-CN | 5-SO₂CF₃ |
| IA-1187 | 2-CN | 5-CN | IA-1188 | 2-CN | 5-SCH₃ |
| IA-1189 | 2-CN | 5-SO₂CH₃ | IA-1190 | 2-CN | 5-OCF₂CF₃ |
| IA-1191 | 2-CN | 5- | IA-1192 | 2-CN | 5- |
| | | OCH₂CHCH₂ | | | OCH₂CH₂OCH₃ |
| IA-1193 | 2-CN | 5-OCH₂CF₃ | IA-1194 | 2-CN | 5-OCH₂CCH |
| IA-1195 | 2-CN | 5-OCH₂- | IA-1196 | 2-CN | 5-C(OH)(CF₃)₂ |
| | | cyclo-C₃H₅ | | | |
| IA-1197 | 2-OCF₂CF₃ | 5-F | IA-1198 | 2-OCF₂CF₃ | 5-Cl |
| IA-1199 | 2-OCF₂CF₃ | 5-Br | IA-1200 | 2-OCF₂CF₃ | 5-CH₃ |
| IA-1201 | 2-OCF₂CF₃ | 5-CH₂CH₃ | IA-1202 | 2-OCF₂CF₃ | 5-C(CH₃)₃ |
| IA-1203 | 2-OCF₂CF₃ | 5-OCH₃ | IA-1204 | 2-OCF₂CF₃ | 5-OCH₂CH₃ |
| IA-1205 | 2-OCF₂CF₃ | 5-CF₃ | IA-1206 | 2-OCF₂CF₃ | 5-CH(CF₃)₂ |
| IA-1207 | 2-OCF₂CF₃ | 5-OC(CH₃)₃ | IA-1208 | 2-OCF₂CF₃ | 5-CF(CF₃)₂ |
| IA-1209 | 2-OCF₂CF₃ | 5-OCF₃ | IA-1210 | 2-OCF₂CF₃ | 5-OCHF₂ |
| IA-1211 | 2-OCF₂CF₃ | 5-SCF₃ | IA-1212 | 2-OCF₂CF₃ | 5-SO₂CF₃ |
| IA-1213 | 2-OCF₂CF₃ | 5-CN | IA-1214 | 2-OCF₂CF₃ | 5-SCH₃ |
| IA-1215 | 2-OCF₂CF₃ | 5-SO₂CH₃ | IA-1216 | 2-OCF₂CF₃ | 5-OCF₂CF₃ |
| IA-1217 | 2-OCF₂CF₃ | 5- | IA-1218 | 2-OCF₂CF₃ | 5- |
| | | OCH₂CHCH₂ | | | OCH₂CH₂OCH₃ |
| IA-1219 | 2-OCF₂CF₃ | 5-OCH₂CF₃ | IA-1220 | 2-OCF₂CF₃ | 5-OCH₂CCH |
| IA-1221 | 2-OCF₂CF₃ | 5-OCH₂- | IA-1222 | 2-OCF₂CF₃ | 5-C(OH)(CF₃)₂ |
| | | cyclo-C₃H₅ | | | |
| IA-1223 | 2- | 5-F | IA-1224 | 2- | 5-Cl |
| | OCH₂CHCH₂ | | | OCH₂CHCH₂ | |
| IA-1225 | 2- | 5-Br | IA-1226 | 2- | 5-CH₃ |
| | OCH₂CHCH₂ | | | OCH₂CHCH₂ | |
| IA-1227 | 2- | 5-CH₂CH₃ | IA-1228 | 2- | 5-C(CH₃)₃ |
| | OCH₂CHCH₂ | | | OCH₂CHCH₂ | |
| IA-1229 | 2- | 5-OCH₃ | IA-1230 | 2- | 5-OCH₂CH₃ |
| | OCH₂CHCH₂ | | | OCH₂CHCH₂ | |
| IA-1231 | 2- | 5-CF₃ | IA-1232 | 2- | 5-CH(CF₃)₂ |
| | OCH₂CHCH₂ | | | OCH₂CHCH₂ | |
| IA-1233 | 2- | 5-OC(CH₃)₃ | IA-1234 | 2- | 5-CF(CF₃)₂ |
| | OCH₂CHCH₂ | | | OCH₂CHCH₂ | |
| IA-1235 | 2- | 5-OCF₃ | IA-1236 | 2- | 5-OCHF₂ |
| | OCH₂CHCH₂ | | | OCH₂CHCH₂ | |
| IA-1237 | 2- | 5-SCF₃ | IA-1238 | 2- | 5-SO₂CF₃ |
| | OCH₂CHCH₂ | | | OCH₂CHCH₂ | |
| IA-1239 | 2- | 5-CN | IA-1240 | 2- | 5-SCH₃ |
| | OCH₂CHCH₂ | | | OCH₂CHCH₂ | |
| IA-1241 | 2- | 5-SO₂CH₃ | IA-1242 | 2- | 5-OCF₂CF₃ |
| | OCH₂CHCH₂ | | | OCH₂CHCH₂ | |
| IA-1243 | 2- | 5- | IA-1244 | 2- | 5- |
| | OCH₂CHCH₂ | OCH₂CHCH₂ | | OCH₂CHCH₂ | OCH₂CH₂OCH₃ |
| IA-1245 | 2- | 5-OCH₂CF₃ | IA-1246 | 2- | 5-OCH₂CCH |
| | OCH₂CHCH₂ | | | OCH₂CHCH₂ | |
| IA-1247 | 2- | 5-OCH₂- | IA-1248 | 2- | 5-C(OH)(CF₃)₂ |
| | OCH₂CHCH₂ | cyclo-C₃H₅ | | OCH₂CHCH₂ | |
| IA-1249 | 2-OCH₂CCH | 5-F | IA-1250 | 2-OCH₂CCH | 5-Cl |
| IA-1251 | 2-OCH₂CCH | 5-Br | IA-1252 | 2-OCH₂CCH | 5-CH₃ |
| IA-1253 | 2-OCH₂CCH | 5-CH₂CH₃ | IA-1254 | 2-OCH₂CCH | 5-C(CH₃)₃ |
| IA-1255 | 2-OCH₂CCH | 5-OCH₃ | IA-1256 | 2-OCH₂CCH | 5-OCH₂CH₃ |
| IA-1257 | 2-OCH₂CCH | 5-CF₃ | IA-1258 | 2-OCH₂CCH | 5-CH(CF₃)₂ |
| IA-1259 | 2-OCH₂CCH | 5-OC(CH₃)₃ | IA-1260 | 2-OCH₂CCH | 5-CF(CF₃)₂ |
| IA-1261 | 2-OCH₂CCH | 5-OCF₃ | IA-1262 | 2-OCH₂CCH | 5-OCHF₂ |
| IA-1263 | 2-OCH₂CCH | 5-SCF₃ | IA-1264 | 2-OCH₂CCH | 5-SO₂CF₃ |
| IA-1265 | 2-OCH₂CCH | 5-CN | IA-1266 | 2-OCH₂CCH | 5-SCH₃ |
| IA-1267 | 2-OCH₂CCH | 5-SO₂CH₃ | IA-1268 | 2-OCH₂CCH | 5-OCF₂CF₃ |
| IA-1269 | 2-OCH₂CCH | 5- | IA-1270 | 2-OCH₂CCH | 5- |
| | | OCH₂CHCH₂ | | | OCH₂CH₂OCH₃ |
| IA-1271 | 2-OCH₂CCH | 5-OCH₂CF₃ | IA-1272 | 2-OCH₂CCH | 5-OCH₂CCH |
| IA-1273 | 2-OCH₂CCH | 5-OCH₂- | IA-1274 | 2-OCH₂CCH | 5-C(OH)(CF₃)₂ |
| | | cyclo-C₃H₅ | | | |
| IA-1275 | 2-OCH₂CF₃ | 5-F | IA-1276 | 2-OCH₂CF₃ | 5-Cl |
| IA-1277 | 2-OCH₂CF₃ | 5-Br | IA-1278 | 2-OCH₂CF₃ | 5-CH₃ |
| IA-1279 | 2-OCH₂CF₃ | 5-CH₂CH₃ | IA-1280 | 2-OCH₂CF₃ | 5-C(CH₃)₃ |
| IA-1281 | 2-OCH₂CF₃ | 5-OCH₃ | IA-1282 | 2-OCH₂CF₃ | 5-OCH₂CH₃ |
| IA-1283 | 2-OCH₂CF₃ | 5-CF₃ | IA-1284 | 2-OCH₂CF₃ | 5-CH(CF₃)₂ |
| IA-1285 | 2-OCH₂CF₃ | 5-OC(CH₃)₃ | IA-1286 | 2-OCH₂CF₃ | 5-CF(CF₃)₂ |
| IA-1287 | 2-OCH₂CF₃ | 5-OCF₃ | IA-1288 | 2-OCH₂CF₃ | 5-OCHF₂ |
| IA-1289 | 2-OCH₂CF₃ | 5-SCF₃ | IA-1290 | 2-OCH₂CF₃ | 5-SO₂CF₃ |
| IA-1291 | 2-OCH₂CF₃ | 5-CN | IA-1292 | 2-OCH₂CF₃ | 5-SCH₃ |
| IA-1293 | 2-OCH₂CF₃ | 5-SO₂CH₃ | IA-1294 | 2-OCH₂CF₃ | 5-OCF₂CF₃ |
| IA-1295 | 2-OCH₂CF₃ | 5- | IA-1296 | 2-OCH₂CF₃ | 5- |
| | | OCH₂CHCH₂ | | | OCH₂CH₂OCH₃ |
| IA-1297 | 2-OCH₂CF₃ | 5-OCH₂CF₃ | IA-1298 | 2-OCH₂CF₃ | 5-OCH₂CCH |
| IA-1299 | 2-OCH₂CF₃ | 5-OCH₂- | IA-1300 | 2-OCH₂CF₃ | 5-C(OH)(CF₃)₂ |
| | | cyclo-C₃H₅ | | | |
| IA-1301 | 2-OCH₂CH₂—OCH₃ | 5-F | IA-1302 | 2-OCH₂CH₂—OCH₃ | 5-Cl |
| IA-1303 | 2-OCH₂CH₂—OCH₃ | 5-Br | IA-1304 | 2-OCH₂CH₂—OCH₃ | 5-CH₃ |
| IA-1305 | 2-OCH₂CH₂—OCH₃ | 5-CH₂CH₃ | IA-1306 | 2-OCH₂CH₂—OCH₃ | 5-C(CH₃)₃ |
| IA-1307 | 2-OCH₂CH₂—OCH₃ | 5-OCH₃ | IA-1308 | 2-OCH₂CH₂—OCH₃ | 5-OCH₂CH₃ |
| IA-1309 | 2-OCH₂CH₂—OCH₃ | 5-CF₃ | IA-1310 | 2-OCH₂CH₂—OCH₃ | 5-CH(CF₃)₂ |
| IA-1311 | 2-OCH₂CH₂—OCH₃ | 5-OC(CH₃)₃ | IA-1312 | 2-OCH₂CH₂—OCH₃ | 5-CF(CF₃)₂ |
| IA-1313 | 2-OCH₂CH₂—OCH₃ | 5-OCF₃ | IA-1314 | 2-OCH₂CH₂—OCH₃ | 5-OCHF₂ |
| IA-1315 | 2-OCH₂CH₂—OCH₃ | 5-SCF₃ | IA-1316 | 2-OCH₂CH₂—OCH₃ | 5-SO₂CF₃ |
| IA-1317 | 2-OCH₂CH₂—OCH₃ | 5-CN | IA-1318 | 2-OCH₂CH₂—OCH₃ | 5-SCH₃ |
| IA-1319 | 2-OCH₂CH₂—OCH₃ | 5-SO₂CH₃ | IA-1320 | 2-OCH₂CH₂—OCH₃ | 5-OCF₂CF₃ |
| IA-1321 | 2-OCH₂CH₂—OCH₃ | 5- | IA-1322 | 2-OCH₂CH₂—OCH₃ | 5- |
| | | OCH₂CHCH₂ | | | OCH₂CH₂OCH₃ |
| IA-1323 | 2-OCH₂CH₂—OCH₃ | 5-OCH₂CF₃ | IA-1324 | 2-OCH₂CH₂—OCH₃ | 5-OCH₂CCH |
| IA-1325 | 2-OCH₂CH₂—OCH₃ | 5-OCH₂- | IA-1326 | 2-OCH₂CH₂—OCH₃ | 5-C(OH)(CF₃)₂ |
| | | cyclo-C₃H₅ | | | |
| IA-1327 | 2-OC(CH₃)₃ | 5-F | IA-1328 | 2-OC(CH₃)₃ | 5-Cl |
| IA-1329 | 2-OC(CH₃)₃ | 5-Br | IA-1330 | 2-OC(CH₃)₃ | 5-CH₃ |
| IA-1331 | 2-OC(CH₃)₃ | 5-CH₂CH₃ | IA-1332 | 2-OC(CH₃)₃ | 5-C(CH₃)₃ |
| IA-1333 | 2-OC(CH₃)₃ | 5-OCH₃ | IA-1334 | 2-OC(CH₃)₃ | 5-OCH₂CH₃ |
| IA-1335 | 2-OC(CH₃)₃ | 5-CF₃ | IA-1336 | 2-OC(CH₃)₃ | 5-CH(CF₃)₂ |
| IA-1337 | 2-OC(CH₃)₃ | 5-OC(CH₃)₃ | IA-1338 | 2-OC(CH₃)₃ | 5-CF(CF₃)₂ |
| IA-1339 | 2-OC(CH₃)₃ | 5-OCF₃ | IA-1340 | 2-OC(CH₃)₃ | 5-OCHF₂ |
| IA-1341 | 2-OC(CH₃)₃ | 5-SCF₃ | IA-1342 | 2-OC(CH₃)₃ | 5-SO₂CF₃ |
| IA-1343 | 2-OC(CH₃)₃ | 5-CN | IA-1344 | 2-OC(CH₃)₃ | 5-SCH₃ |
| IA-1345 | 2-OC(CH₃)₃ | 5-SO₂CH₃ | IA-1346 | 2-OC(CH₃)₃ | 5-OCF₂CF₃ |
| IA-1347 | 2-OC(CH₃)₃ | 5- | IA-1348 | 2-OC(CH₃)₃ | 5- |
| | | OCH₂CHCH₂ | | | OCH₂CH₂OCH₃ |
| IA-1349 | 2-OC(CH₃)₃ | 5-OCH₂CF₃ | IA-1350 | 2-OC(CH₃)₃ | 5-OCH₂CCH |
| IA-1351 | 2-OC(CH₃)₃ | 5-OCH₂- | IA-1352 | 2-OC(CH₃)₃ | 5-C(OH)(CF₃)₂ |
| | | cyclo-C₃H₅ | | | |
| IA-1353 | 2-OCH₂- | 5-F | IA-1354 | 2-OCH₂- | 5-Cl |
| | cyclo-C₃H₅ | | | cyclo-C₃H₅ | |
| IA-1355 | 2-OCH₂- | 5-Br | IA-1356 | 2-OCH₂- | 5-CH₃ |
| | cyclo-C₃H₅ | | | cyclo-C₃H₅ | |
| IA-1357 | 2-OCH₂- | 5-CH₂CH₃ | IA-1358 | 2-OCH₂- | 5-C(CH₃)₃ |
| | cyclo-C₃H₅ | | | cyclo-C₃H₅ | |
| IA-1359 | 2-OCH₂- | 5-OCH₃ | IA-1360 | 2-OCH₂- | 5-OCH₂CH₃ |
| | cyclo-C₃H₅ | | | cyclo-C₃H₅ | |
| IA-1361 | 2-OCH₂- | 5-CF₃ | IA-1362 | 2-OCH₂- | 5-CH(CF₃)₂ |
| | cyclo-C₃H₅ | | | cyclo-C₃H₅ | |
| IA-1363 | 2-OCH₂- | 5-OC(CH₃)₃ | IA-1364 | 2-OCH₂- | 5-CF(CF₃)₂ |
| | cyclo-C₃H₅ | | | cyclo-C₃H₅ | |
| IA-1365 | 2-OCH₂- | 5-OCF₃ | IA-1366 | 2-OCH₂- | 5-OCHF₂ |
| | cyclo-C₃H₅ | | | cyclo-C₃H₅ | |
| IA-1367 | 2-OCH₂- | 5-SCF₃ | IA-1368 | 2-OCH₂- | 5-SO₂CF₃ |
| | cyclo-C₃H₅ | | | cyclo-C₃H₅ | |
| IA-1369 | 2-OCH₂- | 5-CN | IA-1370 | 2-OCH₂- | 5-SCH₃ |
| | cyclo-C₃H₅ | | | cyclo-C₃H₅ | |
| IA-1371 | 2-OCH₂- | 5-SO₂CH₃ | IA-1372 | 2-OCH₂- | 5-OCF₂CF₃ |
| | cyclo-C₃H₅ | | | cyclo-C₃H₅ | |
| IA-1373 | 2-OCH₂- | 5- | IA-1374 | 2-OCH₂- | 5- |
| | cyolo-C₃H₅ | OCH₂CHCH₂ | | cyclo-C₃H₅ | OCH₂CH₂OCH₃ |
| IA-1375 | 2-OCH₂- | 5-OCH₂CF₃ | IA-1376 | 2-OCH₂- | 5-OCH₂CCH |
| | cyclo-C₃H₅ | | | cyclo-C₃H₅ | |
| IA-1377 | 2-OCH₂- | 5-OCH₂- | IA-1378 | 2-OCH₂- | 5-C(OH)(CF₃)₂ |
| | cyolo-C₃H₅ | cyclo-C₃H₅ | | cyclo-C₃H₅ | |
| IA-1379 | 2-F | 6-F | IA-1380 | 2-F | 6-Cl |
| IA-1381 | 2-F | 6-Br | IA-1382 | 2-F | 6-CH₃ |
| IA-1383 | 2-F | 6-OCH₃ | IA-1384 | 2-F | 6-CF₃ |
| IA-1385 | 2-F | 6-OCF₃ | IA-1386 | 2-F | 6-SCH₃ |
| IA-1387 | 2-F | 6-OCHF₂ | IA-1388 | 2-Cl | 6-F |
| IA-1389 | 2-Cl | 6-Cl | IA-1390 | 2-Cl | 6-Br |
| IA-1391 | 2-Cl | 6-CH₃ | IA-1392 | 2-Cl | 6-OCH₃ |
| IA-1393 | 2-Cl | 6-CF₃ | IA-1394 | 2-Cl | 6-OCF₃ |
| IA-1395 | 2-Cl | 6-SCH₃ | IA-1396 | 2-Cl | 6-OCHF₂ |
| IA-1397 | 2-Br | 6-F | IA-1398 | 2-Br | 6-Cl |
| IA-1399 | 2-Br | 6-Br | IA-1400 | 2-Br | 6-CH₃ |
| IA-1401 | 2-Br | 6-OCH₃ | IA-1402 | 2-Br | 6-CF₃ |
| IA-1403 | 2-Br | 6-OCF₃ | IA-1404 | 2-Br | 6-SCH₃ |
| IA-1405 | 2-Br | 6-OCHF₂ | IA-1406 | 2-CH₃ | 6-F |
| IA-1407 | 2-CH₃ | 6-Cl | IA-1408 | 2-CH₃ | 6-Br |
| IA-1409 | 2-CH₃ | 6-CH₃ | IA-1410 | 2-CH₃ | 6-OCH₃ |
| IA-1411 | 2-CH₃ | 6-CF₃ | IA-1412 | 2-CH₃ | 6-OCF₃ |
| IA-1413 | 2-CH₃ | 6-SCH₃ | IA-1414 | 2-CH₃ | 6-OCHF₂ |
| IA-1415 | 2-OCH₃ | 6-F | IA-1416 | 2-OCH₃ | 6-Cl |
| IA-1417 | 2-OCH₃ | 6-Br | IA-1418 | 2-OCH₃ | 6-CH₃ |
| IA-1419 | 2-OCH₃ | 6-OCH₃ | IA-1420 | 2-OCH₃ | 6-CF₃ |
| IA-1421 | 2-OCH₃ | 6-OCF₃ | IA-1422 | 2-OCH₃ | 6-SCH₃ |
| IA-1423 | 2-OCH₃ | 6-OCHF₂ | IA-1424 | 2-CF₃ | 6-F |
| IA-1425 | 2-CF₃ | 6-Cl | IA-1426 | 2-CF₃ | 6-Br |
| IA-1427 | 2-CF₃ | 6-CH₃ | IA-1428 | 2-CF₃ | 6-OCH₃ |
| IA-1429 | 2-CF₃ | 6-CF₃ | IA-1430 | 2-CF₃ | 6-OCF₃ |
| IA-1431 | 2-CF₃ | 6-SCH₃ | IA-1432 | 2-CF₃ | 6-OCHF₂ |
| IA-1433 | 2-SCH₃ | 6-F | IA-1434 | 2-SCH₃ | 6-Cl |
| IA-1435 | 2-SCH₃ | 6-Br | IA-1436 | 2-SCH₃ | 6-CH₃ |
| IA-1437 | 2-SCH₃ | 6-OCH₃ | IA-1438 | 2-SCH₃ | 6-CF₃ |
| IA-1439 | 2-SCH₃ | 6-OCF₃ | IA-1440 | 2-SCH₃ | 6-SCH₃ |
| IA-1441 | 2-SCH₃ | 6-OCHF₂ | IA-1442 | 2-OCF₃ | 6-F |
| IA-1443 | 2-OCF₃ | 6-Cl | IA-1444 | 2-OCF₃ | 6-Br |
| IA-1445 | 2-OCF₃ | 6-CH₃ | IA-1446 | 2-OCF₃ | 6-OCH₃ |
| IA-1447 | 2-OCF₃ | 6-CF₃ | IA-1448 | 2-OCF₃ | 6-OCF₃ |
| IA-1449 | 2-OCF₃ | 6-SCH₃ | IA-1450 | 2-OCF₃ | 6-OCHF₂ |
| IA-1451 | 2-OCHF₂ | 6-F | IA-1452 | 2-OCHF₂ | 6-Cl |
| IA-1453 | 2-OCHF₂ | 6-Br | IA-1454 | 2-OCHF₂ | 6-CH₃ |
| IA-1455 | 2-OCHF₂ | 6-OCH₃ | IA-1456 | 2-OCHF₂ | 6-CF₃ |
| IA-1457 | 2-OCHF₂ | 6-OCF₃ | IA-1458 | 2-OCHF₂ | 6-SCH₃ |
| IA-1459 | 2-OCHF₂ | 6-OCHF₂ | IA-1460 | 2-Cl | 4- |
| | | | | | C(CH₃)═NOCH₃ |

In the intermediates of formula II of the present invention, the variables have the same preferred meanings as described above for compounds of formula I.

The compounds of the formula I are especially suitable for efficiently combating the following pests:

insects from the order of the lepidopterans (Lepidoptera), for example Agrotis ypsilon, Agrotis segetum, Alabama argillacea, Anilcarsia gemmatalls, Argyresthia conjugella, Autographa gamma, Bupalus piniarius, Cacoecia murinana, Capua reticulana, Chematobia brumata, Choristoneura fumiferana, Choristoneura occidentalis, Cirphis unipuncta, Cydia pomonella, Dendrolimus pini, Diaphania nitidalls, Diafraea grandiosella, Earias insulana, Elasmopalpus lignosellus, Eupoecilla ambiguella, Evetria bouliana, Feltia subterranea, Galleria mellonella, Grapholitha funebrana, Grapholitha molesta, Hellothis armigera, Hellothis virescens, Hellothis zea, Hellula undalis, Hibernia defollaria, Hyphantria cunea, Hyponomeuta malinellus, Kelferia lycopersicella, Lambdina fiscellaria, Laphygma exigua, Leucoptera coffeella, Leucoptera scilella, Lithocolletis blancardella, Lobesia botrana, Loxostege sticticalls, Lymantria dispar, Lymantria monacha, Lyonetia clerkella, Malacosoma neustria, Mamestra brassicae, Orgyia pseudotsugata, Ostrinia nubilalis, Panolis flammea, Pectilnophora gossypiella, Peridroma saucia, Phalera bucephala, Phthommaea operculella, Phyllocnistis citrella, Pieris brassicae, Plathypena scabra, Plutella xylostella, Pseudoplusia includens, Rhyacionia frusfrana, Scrobipalpula absoluta, Siftotroga cerealella, Sparganothis pilleriana, Spodoptera frugiperda, Spodoptera liltoralls, Spodoptera litura, Thaumatopoea pityocampa, Tortrix viridana, Trichoplusia ni and Zeiraphera canadensis,
beetles (Coleoptera), for example Agrilus sinuatus, Agriotes lineatus, Agriotes obscurus, Amphimallus solstitialis, Anisandrus dispar, Anthonomus grandis, Anthonomus pomorum, Aphthona euphoridae, Athous haemorrhoidalis, Atomaria linearis, Blastophagus piniperda, Blltophaga undata, Bruchus rufimanus, Bruchus pisorum, Bruchus lentils, Byctiscus betulae, Cassida nebulosa, Cerotoma trifurcata, Cetonia aurata, Ceuthorrhynchus assimilis, Ceuthorrhynchus napi, Chaetocnema tibialis, Conoderus vespertinus, Crioceris asparagi, Ctenicera ssp., Diabrotica longicornis, Diabrotica semipunctata, Diabrotica 12-punctata Diabrotica speciosa, Diabrotica virgifera, Epilachna varivestis, Epitrix hirtipennis, Eutinobothrus brasiliensis, Hylobius abietis, Hypera brunneipennis, Hypera postica, Ips typographus, Lema bilineata, Lema melanopus, Leptinotarsa decemlineata, Limonius californicus, Lissorhoptrus oryzophilus, Melanotus communis, Meligethes aeneus, Melolontha hippocastani, Melolontha melolontha, Oulema oryzae, Ortiorrhynchus sulcatus, Otiorrhynchus ovatus, Phaedon cochleariae, Phyllobius pyri, Phyllotreta chrysocephala, Phyllophaga sp., Phyllopertha horticola, Phyllotreta nemorum, Phyllotreta striolata, Popillia japonica, Sitona lineatus and Sitophilus granaria,
flies, mosquitoes (Diptera), e.g. Aedes aegypti, Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrimaculatus, Calliphora vicina, Ceratitis capitata, Chrysomya bezziana, Chrysomya hominivorax, Chrysomya macellaria, Chrysops discalis, Chrysops silacea, Chrysops atlanticus, Cochliomyla hominivorax, Contarnia sorghicola Cordylobia anthropophaga, Culicoides furens, Culex pipiens, Culex nigripalpus, Culex quinquefasciatus, Culex tarsalls, Culiseta inornata, Culiseta melanura, Dacus cucurbitae, Dacus oleae, Dasineura brassicae, Delia antique, Della coarctata, Della platura, Della radicum, Dermatobia hominis, Fannia canicularis, Geomyza Tripunctata, Gaster ophilus intestinalis, Glossina morsitans, Glossina palpalis, Glossina fuscipes, Glossina tachinoides, Haematobia irritans, Haplodiplosis equestris, Hippelates spp., Hylemyia platura, Hypoderma lineata, Leptoconops torrens, Liriomyza sativae, Liriomyza trifolii, Lucilla caprina, Lucilia cuprina, Lucilia sericata, Lycoria pectoralls, Mansonia titillanus, Mayetiola destructor, Musca domestica, Muscina stabulans, Oestrus ovis, Opomyza florum, Oscinella frit, Pegomya hysocyami, Phorbia antiqua, Phorbia brassicae, Phorbia coarctata, Phlebotomus argentipes, Psorophora columbiae, Psila rosae, Psorophora discolor, Prosimullum mixtum, Rhagoletis cerasi, Rhagoletis pomonella, Sarcophaga haemorrhoidalls, Sarcophaga sp., Simullum vlttatum, Stomoxys calcitrans, Tabanus bovinus, Tabanus atratus, Tabanus lineola, and Tabanus similis, Tipula oleracea, and Tipula paludosa
thrips (Thysanoptera), e.g. Dichromothrips corbetti, Dichromothrips ssp, Frankliniella fusca, Frankliniella occidentalls, Frankliniella tritici, Scirothrips citri; Thrips oryzae, Thrips palmi and Thrips tabaci,
termites (Isoptera), e.g. Calotermes flavicollis, Leucotermes flavipes, Heterotermes aureus, Reticulilermes flavipes, Reticuiftermes virginicus, Reticultermes lucifugus, Termes natalensis, and Coptotermes formosanus,
cockroaches (Blattaria-Blattodea), e.g. Blaftella germanica, Blattella asahinae, Periplaneta americana, Periplaneta japonica, Periplaneta brunnea, Periplaneta fuligginosa, Periplaneta australasiae, and Blafta orientalis,
true bugs (Hemiptera), e.g. Acrosternum hilare, Blissus leucopterus, Cyrtopeltis notatus, Dysdercus cingulatus, Dysdercus intermedius, Eurygaster integriceps, Euschistus impictivenfris, Leptoglossus phyllopus, Lygus lineolaris, Lygus pratensis, Nezara viridula, Piesma quadrata, Solubea insularis, Thyanta perdilor, Acyrthosiphon onobrychis, Adelges laricis, Aphidula nasturtii, Aphis fabae, Aphis forbesi, Aphis pomi, Aphis gossypii, Aphis grossulariae, Aphis schneideri, Aphis spiraecola, Aphis sambuci, Acyrthosiphon pisum, Aulacorthum solani, Bemisia argentifolii, Brachycaudus cardui, Brachycaudus hellchrysi, Brachycaudus persicae, Brachycaudus prunicola, Brevicoryne brassicae, Capilophorus horni, Cerosipha gossypi, Chaetosiphon fragaefolli, Cryptomyzus ribis, Dreyfusia nordmannianae, Dreyfusia piceae, Dysaphis radicola, Dysaulacorthum pseudosolani, Dysaphis plantaginea, Dysaphis pyri, Empoasca fabae, Hyalopterus pruni, Hyperomyzus lactucae, Macrosiphum avenae, Macrosiphum euphorbiae, Macrosiphon rosae, Megoura viciae, Melanaphis pyrarius, Metopolophium dihodum, Myzus persicae, Myzus ascalonicus, Myzus cerasi, Myzus varians, Nasonovia ribis-nigri, Nilaparvata lugens, Pemphigus bursarius, Perkinsiella saccharicida, Phorodon humuli, Psylla mall, Psylla piri, Rhopalomyzus ascalonicus, Rhopalosiphum maidis, Rhopalosiphum padi Rhopalosiphum insertum, Sappaphis mala, Sappaphis mali, Schizaphis graminum, Schizoneura lanuginosa, Sitobion avenae, Trialeurodes vaporariorum, Toxoptera aurantiiand, Viteus vitifolii, Cimex lectularlus, Cimex hemipterus, Reduvius senilis, Triatoma spp., and Arilus critatus.
ants, bees, wasps, sawflies (Hymenoptera), e.g. Athalia rosae, Atta cephalotes, Afta capiguara, Afta cephalotes, Afta laevigata, Afta robusta, Afta sexdens, Atta texana, Crematogaster spp., Hoplocampa minuta, Hoplocampa testudinea, Monomorium pharaonis, Solenopsis geminate, Solenopsis invicta, Solenopsis richter, Solenopsis xyloni, Pogonomyrmex barbatus, Pogonomyrmex califormicus, Pheidole megacephala, Dasymutilla occidentalis, Bombus spp. Vespula squamosa, Paravespula vulgaris, Paravespula pennsylvanica, Paravespula germanica, Dollchovespula maculata, Vespa crabro, Polistes rubiginosa, Camponotus floridanus, and Linepithema humile,
crickets, grasshoppers, locusts (Orthoptera), e.g. Acheta domestica, Gryllotalpa gryllotalpa, Locusta migratoria, Melanoplus bivittatus, Melanoplus femurrubrum, Melanoplus mexicanus, Melanoplus sanguinipes, Melanoplus spretus, Nomadacris septemfasciata, Schistocerca americana, Schistocerca gregaria, Dociostaurus maroccanus, Tachycines asynamorus, Oedaleus senegalensis, Zonozerus variegatus, Hieroglyphus daganensis, Kraussaria angulifera, Calliptamus italilcus, Chortoicetes terminifera, and Locustana pardalina,
Arachnoidea, such as arachnids (Acarina), e.g. of the families Argasidae, Ixodidae and Sarcoptidae, such as Amblyomma americanum, Amblyomma variegatum, Ambryomma maculatum, Argas persicus, Boophilus annulatus, Boophilus decoloratus, Boophilus nmicroplus, Dermacentor silvarum, Dermacentor andersoni, Dermacentor variabills, Hyalomma truncatum, Ixodes ricinus, Ixodes rubicundus, Ixodes scapularis, Ixodes holocyclus, Ixodes pacifcus, Ornithodorus moubata, Ornithodorus hermsi, Ornithodorus turicata, Ornithonyssus bacoti, Otobius megnini, Dermanyssus gallinae, Psoroptes ovis, Rhipicephalus sanguineus, Rhipicephalus appendiculatus, Rhipicephalus evertsi, Sarcoptes scabiei; and Eriophyidae spp. such as Aculus schlechtendai, Phyllocoptrata oleivora and Eriophyes sheldoni, Tarsonemidae spp. such as Phytonemus pallidus and Polyphagotarsonemus latus, Tenuipalpidae spp. such as Brevipalpus phoenicis; Tetranychidae spp. such as Tetranychus cinnabarinus, Tetranychus kanzawai, Tetranychus pacificus, Tetranychus telarius and Tetranychus urticae, Panonychus ulmi, Panonychus citri, and Oligonychus pratensis; Araneida, e.g. Lafrodectus mactans, and Loxosceles reclusa,
fleas (Siphonaptera), e.g. Ctenocephalides felis, Ctenocephalides canis, Xenopsylla cheopis, Pulex irritans, Tunga penefrans, and Nosopsyllus fasciatus,
silverfish, firebrat (Thysanura), e.g. Lepisma saccharina and Thermobia domestica,
centipedes (Chilopoda), e.g. Scutigera coleopfrata,
millipedes (Diplopoda), e.g. Narceus spp.,
earwigs (Dermaptera), e.g. forficula auricularia,
lice (Phthiraptera), e.g. Pediculus humanus capitis, Pediculus humanus corporis, Pthirus pubis, Haematopinus eurysternus, Haematopinus suis, Linognathus vituli, Bovicola bovis, Menopon gallinae, Menacanthus stramineus and Solenopotes capillatus,
nematodes, especially plant parasitic nematodes such as root knot nematodes, Meloidogyne hapla, Meloidogyne incognita, Meloidogyne javanica, and other Meloidogyne species; cyst-forming nematodes, Globodera rostochiensis and other Globodera species; Heterodera avenae, Heterodera glycines, Heterodera schachtii, Heterodera trifolii, and other Heterodera species; Seed gall nematodes, Anguina species; Stem and foliar nematodes, Aphelenchoides species; Sting nematodes, Belonolaimus longicaudatus and other Belonolaimus species; Pine nematodes, Bursaphelenchus xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Lesion nematodes, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species; Dagger nematodes, Xiphinema species; and other plant parasitic nematode species.

The formulations are prepared in a known manner (see e.g. for review U.S. Pat. No. 3,060,084, EP-A 707 445 (for liquid concentrates), Browning, “Agglomeration”, Chemical Engineering, Dec. 4, 1967, 147-48, Perry's Chemical Engineer's Handbook, 4th Ed., McGraw-Hill, New York, 1963, pages 8-57 and et seq. WO 91/13546, U.S. Pat. No. 4,172,714, U.S. Pat. No. 4,144,050, U.S. Pat. No. 3,920,442, U.S. Pat. No. 5,180,587, U.S. Pat. No. 5,232,701, U.S. Pat. No. 5,208,030, GB 2,095,558, U.S. Pat. No. 3,299,566, Klingman, Weed Control as a Science, John Wiley and Sons, Inc., New York, 1961, Hance et al., Weed Control Handbook, 8th Ed., Blackwell Scientific Publications, Oxford, 1989 and Mollet, H., Grubemann, A., Formulation technology, Wiley VCH Verlag GmbH, Weinheim (Germany), 2001, 2. D. A. Knowles, Chemistry and Technology of Agrochemical Formulations, Kluwer Academic Publishers, Dordrecht, 1998 (ISBN 0-7515-0443-8), for example by extending the active compound with auxiliaries suitable for the formulation of agrochemicals, such as solvents and/or carriers, if desired emulsifiers, surfactants and dispersants, preservatives, anti-foaming agents, anti-freezing agents, for seed treatment formulation also optionally colorants and binders.

Examples of suitable solvents are water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used.

Examples of suitable carriers are ground natural minerals (for example kaolins, clays, talc, chalk) and ground synthetic minerals (for example highly disperse silica, silicates).

Suitable emulsifiers are nonionic and anionic emulsifiers (for example polyoxyethylene fatty alcohol ethers, alkylsulfonates and arylsulfonates).

Examples of dispersants are lignin-sulfite waste liquors and methylcellulose.

Suitable surfactants used are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenol ether, ethoxylated isooctylphenol, octylphenol, nonylphenol, alkylphenol polyglycol ethers, tributylphenyl polyglycol ether, tristearylphenyl polyglycol ether, alkylaryl polyether alcohols, alcohol and fatty alcohol ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, highly polar solvents, for example dimethyl sulfoxide, N-methylpyrrolidone or water.

Also anti-freezing agents such as glycerin, ethylene glycol, propylene glycol and bactericides such as can be added to the formulation.

Suitable antifoaming agents are for example antifoaming agents based on silicon or magnesium stearate.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nut-shell meal, cellulose powders and other solid carriers.

In general, the formulations comprise from 0.01 to 95% by weight, preferably from 0.1 to 90% by weight, of the active compound(s). In this case, the active compound(s) are employed in a purity of from 90% to 100% by weight, preferably 95% to 100% by weight (according to NMR spectrum).

The compounds of formula I can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; they are intended to ensure in each case the finest possible distribution of the active compound(s) according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. However, it is also possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1% per weight.

The active compound(s) may also be used successfully in the ultra-low-volume process (ULV), it being possible to apply formulations comprising over 95% by weight of active compound, or even to apply the active compound without additives.

The following are examples of formulations: 1. Products for dilution with water for foliar applications. For seed treatment purposes, such products may be applied to the seed diluted or undiluted.

### A) Water-Soluble Concentrates (SL, LS)

10 parts by weight of the active compound(s) are dissolved in 90 parts by weight of water or a water-soluble solvent. As an alternative, wetters or other auxiliaries are added. The active compound(s) dissolves upon dilution with water, whereby a formulation with 10% (w/w) of active compound(s) is obtained.

### B) Dispersible Concentrates (DC)

20 parts by weight of the active compound(s) are dissolved in 75 parts by weight of cyclohexanone with addition of 10 parts by weight of a dispersant, for example polyvinylpyrrolidone. Dilution with water gives a dispersion, whereby a formulation with 20% (w/w) of active compound(s) is obtained.

### C) Emulsifiable Concentrates (EC)

15 parts by weight of the active compound(s) are dissolved in 75 parts by weight of xylene with addition of calcium dodecylbenzenesulfonate and castor oil ethoxylate (in each case 5 parts by weight). Dilution with water gives an emulsion, whereby a formulation with 15% (w/w) of active compound(s) is obtained.

### D) Emulsions (EW, EO, ES)

40 parts by weight of the active compound(s) are dissolved in 35 parts by weight of xylene with addition of calcium dodecylbenzenesulfonate and castor oil ethoxylate (in each case 5 parts by weight). This mixture is introduced into 30 parts by weight of water by means of an emulsifier machine (e.g. Ultraturrax) and made into a homogeneous emulsion. Dilution with water gives an emulsion, whereby a formulation with 25% (w/w) of active compound(s) is obtained.

### E) Suspensions (SC, OD, FS)

In an agitated ball mill, 20 parts by weight of the active compound(s) are comminuted with addition of 10 parts by weight of dispersants, wetters and 70 parts by weight of water or of an organic solvent to give a fine active compound(s) suspension. Dilution with water gives a stable suspension of the active compound(s), whereby a formulation with 20% (w/w) of active compound(s) is obtained.

### F) Water-Dispersible Granules and Water-Soluble Granules (WG, SG)

50 parts by weight of the active compound(s) are ground finely with addition of 50 parts by weight of dispersants and wetters and made as water-dispersible or water-soluble granules by means of technical appliances (for example extrusion, spray tower, fluidized bed). Dilution with water gives a stable dispersion or solution of the active compound(s), whereby a formulation with 50% (w/w) of active compound(s) is obtained.

### G) Water-Dispersible Powders and Water-Soluble Powders (WP, SP, SS, WS)

75 parts by weight of the active compound(s) are ground in a rotor-stator mill with addition of 25 parts by weight of dispersants, wetters and silica gel. Dilution with water gives a stable dispersion or solution of the active compound(s), whereby a formulation with 75% (w/w) of active compound(s) is obtained.

2. Products to be applied undiluted for foliar applications. For seed treatment purposes, such products may be applied to the seed diluted or undiluted.

### H) Dustable Powders (DP, DS)

5 parts by weight of the active compound(s) are ground finely and mixed intimately with 95 parts by weight of finely divided kaolin. This gives a dustable product having 5% (w/w) of active compound(s)

I) Granules (GR, FG, GG, MG)

0.5 part by weight of the active compound(s) is ground finely and associated with 95.5 parts by weightof carriers, whereby a formulation with 0.5% (w/w) of active compound(s) is obtained. Current methods are extrusion, spray-drying or the fluidized bed. This gives granules to be applied undiluted for foliar use.

J) ULV Solutions (UL, LS)

10 parts by weight of the active compound(s) are dissolved in 90 parts by weight of an organic solvent, for example xylene. This gives a product having 10% (w/w) of active compound(s), which is applied undiluted for foliar use.

Various types of oils, wetters, adjuvants, herbicides, fungicides, other pesticides, or bactericides may be added to the active ingredients, if appropriate just immediately prior to use (tank mix). These agents usually are admixed with the agents according to the invention in a weight ratio of 1:10 to 10:1.

The compounds of formula I are effective through both contact and ingestion.

The compounds of formula I are also suitable for the protection of the seed, plant propagules and the seedlings' roots and shoots, preferably the seeds, against soil pests and also for the treatment plant seeds which tolerate the action of herbicides or fungicides or insecticides owing to breeding, including genetic engineering methods.

Conventional seed treatment formulations include for example flowable concentrates FS, solutions LS, powders for dry treatment DS, water dispersible powders WS or granules for slurry treatment, water soluble powders SS and emulsion ES. Application to the seeds is carried out before sowing, either directly on the seeds.

The seed treatment application of the compounds of formula I or formulations containing them is carried out by spraying or dusting the seeds before sowing of the plants and before emergence of the plants.

The invention also relates to the propagation product of plants, and especially the treated seed comprising, that is, coated with and/or containing, a compound of formula I or a composition comprising it. The term “coated with and/or containing” generally signifies that the active ingredient is for the most part on the surface of the propagation product at the time of application, although a greater or lesser part of the ingredient may penetrate into the propagation product, depending on the method of application. When the said propagation product is (re)planted, it may absorb the active ingredient.

The seed comprises the inventive compounds or compositions comprising them in an amount of from 0.1 g to 10 kg per 100 kg of seed.

Compositions of this invention may also contain other active ingredients, for example other pesticides, insecticides, herbicides, fertilizers such as ammonium nitrate, urea, potash, and superphosphate, phytotoxicants and plant growth regulators, safeners and nematicides. These additional ingredients may be used sequentially or in combination with the above-described compositions, if appropriate also added only immediately prior to use (tank mix). For example, the plant(s) may be sprayed with a composition of this invention either before or after being treated with other active ingredients.

The following list of pesticides together with which the compounds according to the invention can be used, is intended to illustrate the possible combinations, but not to impose any limitation:

A.1. Organo(thio)phosphates: acephate, azamethiphos, azinphos-ethyl, azinphosmethyl, chlorethoxyfos, chlorfenvinphos, chlormephos, chlorpyrifos, chlorpyrifosmethyl, coumaphos, cyanophos, demeton-5-methyl, diazinon, dichlorvos/DDVP, dicrotophos, dimethoate, dimethylvinphos, disulfoton, EPN, ethion, ethoprophos, famphur, fenamiphos, fenitrothion, fenthion, flupyrazophos, fosthiazate, heptenophos, isoxathion, malathion, mecarbam, methamidophos, methidathion, mevinphos, monocrotophos, naled, omethoate, oxydemeton-methyl, parathion, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, trichlorfon, vamidothion;

A.2. Carbamates: aldicarb, alanycarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, trimethacarb, XMC, xylylcarb, triazamate; A.3. Pyrethroids: acrinathrin, allethrin, d-cis-trans allethrin, d-trans allethrin, bifenthrin, bioallethrin, bioallethrin S-cylclopentenyl, bioresmethrin, cycloprothrin, cyfluthrin, beta-, yfluthrin, cyhalothrin, lambda-cyhalothrin, gamma-cyhalothrin, cypermethrin, alphacypermethrin, beta-cypermethrin, theta-cypermethrin, zeta-cypermethrin, cyphenothrin, deltamethrin, empenthrin, esfenvalerate, etofenprox, fenpropathrin, fenvalerate, flucythrinate, flumethrin, tau-fluvalinate, halfenprox, imiprothrin, permethrin, phenothrin, prallethrin, resmethrin, RU 15525, silafluofen, tefluthrin, tetramethrin, tralomethrin, transfluthrin, ZXI 8901;

A.4. Juvenile hormone mimics: hydroprene, kinoprene, methoprene, fenoxycarb, pyriproxyfen;

A.5. Nicotinic receptor agonists/antagonists compounds: acetamiprid, bensultap, cartap hydrochloride, clothianidin, dinotefuran, imidacloprid, thiamethoxam, nitenpyram, nicotine, spinosad (allosteric agonist), thiacloprid, thiocyclam, thiosultap-sodium, and AKD1022.

A.6. GABA gated chloride channel antagonist compounds: chlordane, endosulfan, gamma-HCH (lindane); acetoprole, ethiprole, fipronil, pyrafluprole, pyriprole, vaniliprole, the phenylpyrazole compound of formula □¹

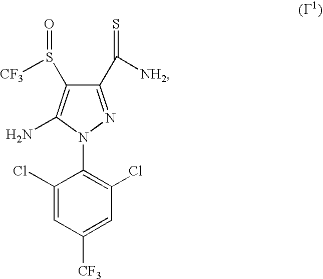

A.7. Chloride channel activators: abamectin, emamectin benzoate, milbemectin, lepimectin;

A.8. METI I compounds: fenazaquin, fenpyroximate, pyrimidifen, pyridaben, tebufenpyrad, tolfenpyrad, flufenerim, rotenone;

A.9. METI II and III compounds: acequinocyl, fluacyprim, hydramethylnon;

A.10. Uncouplers of oxidative phosphorylation: chlorfenapyr, DNOC;

A.11. Inhibitors of oxidative phosphorylation: azocyclotin, cyhexatin, diafenthiuron, fenbutatin oxide, propargite, tetradifon;

A.12. Moulting disruptors: cyromazine, chromafenozide, halofenozide, methoxyfenozide, tebufenozide;

A. 13. Synergists: piperonyl butoxide, tribufos;

A.14. Sodium channel blocker compounds: indoxacarb, metaflumizone;

A.15. Fumigants: methyl bromide, chloropicrin sulfuryl fluoride;

A.16. Selective feeding blockers: crylotie, pymetrozine, flonicamid;

A.17. Mite growth inhibitors: clofentezine, hexythiazox, etoxazole;

A.18. Chitin synthesis inhibitors: buprofezin, bistrifluoron, chlorfluazuron, diflubenzuron, flucycloxuron, flufenoxuron, hexaflumuron, lufenuron, novaluron, noviflumuron, teflubenzuron, triflumuron;

A.19. Lipid biosynthesis inhibitors: spirodiclofen, spiromesifen, spirotetramat;

A.20. octapaminergic agonsits: amitraz;

A.21. ryanodine receptor modulators: flubendiamide;

A.22. Various: aluminium phosphide, amidoflumet, benclothiaz, benzoximate, bifenazate, borax, bromopropylate, cyanide, cyenopyrafen, cyflumetofen, chinomethionate, dicofol, fluoroacetate, phosphine, pyridalyl, pyrifluquinazon, sulfur, tartar emetic;

A.23. N—R′-2,2-dihalo-1-R″cyclo-propanecarboxamide-2-(2,6-dichloro-α,α,α-tri-fluoro-p-tolyl)hydrazone or N—R′-2,2-di(R′″)propionamide-2-(2,6-dichloro-α,α,α-trifluoro-p-tolyl)-hydrazone, wherein R′ is methyl or ethyl, halo is chloro or bromo, R″ is hydrogen or methyl and R′″ is methyl or ethyl;

A.24. Anthranilamides: chloranthraniliprole, the compound of formula Γ²

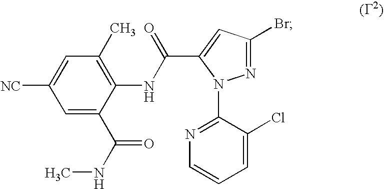

A.25. Malononitrile compounds: CF₃(CH₂)₂C(CN)₂CH₂(CF₂)₃CF₂H, CF₃(CH₂)₂C(CN)₂CH₂(CF₂)₅CF₂H, CF₃(CH₂)₂C(CN)₂(CH₂)₂C(CF₃)₂F, CF₃(CH₂)₂C(CN)₂(CH₂)₂(CF₂)₃CF₃, CF₂H(CF₂)₃CH₂C(CN)₂CH₂(CF₂)₃CF₂H, CF₃(CH₂)₂C(CN)₂CH₂(CF₂)₃CF₃, CF₃(CF₂)₂CH₂C(CN)₂CH₂(CF₂)₃CF₂H, CF₃CF₂CH₂C(CN)₂CH₂(CF₂)₃CF₂H, 2-(2,2,3,3,4,4,5,5-octafluoropentyl)-2-(3,3,4,4,4-pentafluorobutyl)-malonodinitrile, and CF₂HCF₂CF₂CF₂CH₂C(CN)₂CH₂CH₂CF₂CF₃;

A.26. Microbial disruptors: Bacillus thuringiensis subsp. Israelensi, Bacillus sphaericus, Bacillus thuringiensis subsp. Aizawai, Bacillus thuringiensis subsp. Kurstaki, Bacillus thuringiensis subsp. Tenebrionis;

The commercially available compounds of the group A may be found in The Pesticide Manual, 13^{th} Edition, British Crop Protection Council (2003) among other publications.

Thioamides of formula Γ¹ and their preparation have been described in WO 98/28279. Lepimectin is known from Agro Project, PJB Publications Ltd, November 2004. Benclothiaz and its preparation have been described in EP-A1 454621. Methidathion and Paraoxon and their preparation have been described in Farm Chemicals Handbook, Volume 88, Meister Publishing Company, 2001. Acetoprole and its preparation have been described in WO 98/28277. Metaflumizone and its preparation have been described in EP-A1462 456. Flupyrazofos has been described in Pesticide Science 54, 1988, p. 237-243 and in U.S. Pat. No. 4,822,779. Pyrafluprole and its preparation have been described in JP 2002193709 and in WO 01/00614. Pyriprole and its preparation have been described in WO 98/45274 and in U.S. Pat. No. 6,335,357. Amidoflumet and its preparation have been described in U.S. Pat. No. 6,221,890 and in JP 21010907. Flufenerim and its preparation have been described in WO 03/007717 and in WO 03/007718. AKD 1022 and its preparation have been described in U.S. Pat. No. 6,300,348. Chloranthraniliprole has been described in WO 01/70671, WO 03/015519 and WO 05/118552. Anthranilamide derivatives of formula Γ² have been described in WO 01/70671, WO 04/067528 and WO 05/118552. Cyflumetofen and its preparation have been described in WO 04/080180. The aminoquinazolinone compound pyrifluquinazon has been described in EP A 109 7932. The malononitrile compounds CF₃(CH₂)₂C(CN)₂CH₂(CF₂)₃CF₂H, CF₃(CH₂)₂C(CN)₂CH₂(CF₂)₅CF₂H, CF₃(CH₂)₂C(CN)₂(CH₂)₂C(CF₃)₂F, CF₃(CH₂)₂C(CN)₂(CH₂)₂(CF₂)₃CF₃, CF₂H(CF₂)₃CH₂C(CN)₂CH₂(CF₂)₃CF₂H, CF₃(CH₂)₂C(CN)₂CH₂(CF₂)₃CF₃, CF₃(CF₂)₂CH₂C(CN)₂CH₂(CF₂)₃CF₂H, CF₃CF₂CH₂C(CN)₂CH₂(CF₂)₃CF₂H, 2-(2,2,3,3,4,4,5,5-octafluoropentyl)-2-(3,3,4,4,4-pentafluorobutyl)-malonodinitrile, and CF₂HCF₂CF₂CF₂CH₂C(CN)₂CH₂CH₂CF₂CF₃ have been described in WO 05/63694.

The insects, acarids or nematodes may be controlled by contacting the target parasite/pest, its food supply, habitat, breeding ground or its locus with a pesticidally effective amount of compounds of or compositions of formula I.

“Locus” means a habitat, breeding ground, plant, seed, soil, area, material or environment in which a pest or parasite is growing or may grow.

In general, “pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions such as desired pesticidal effect and duration, weather, target species, locus, mode of application, and the like.

The compounds or compositions of the invention can also be applied preventively to places at which occurrence of the pests is expected.

The compounds of formula I may also be used to protect growing plants from attack or infestation by pests by contacting the plant with a pesticidally effective amount of compounds of formula I. As such, “contacting” includes both direct contact (applying the compounds/compositions directly on the pest and/or plant—typically to the foliage, stem or roots of the plant) and indirect contact (applying the compounds/compositions to the locus of the pest and/or plant).

In the case of soil treatment or of application to the pests dwelling place or nest, the quantity of active ingredient ranges from 0.0001 to 500 g per 100 m², preferably from 0.001 to 20 g per 100 m².

For use in treating crop plants, the rate of application of the active ingredients of this invention may be in the range of 0.1 g to 4000 g per hectare, desirably from 25 g to 600 g per hectare, more desirably from 50 g to 500 g per hectare.

Compounds of formula I and compositions comprising them can also be used for controlling and preventing infestations and infections in animals including warm-blooded animals (including humans) and fish. They are for example suitable for controlling and preventing infestations and infections in mammals such as cattle, sheep, swine, camels, deer, horses, pigs, poultry, rabbits, goats, dogs and cats, water buffalo, donkeys, fallow deer and reindeer, and also in fur-bearing animals such as mink, chinchilla and raccoon, birds such as hens, geese, turkeys and ducks and fish such as fresh- and salt-water fish such as trout, carp and eels.

Infestations in warm-blooded animals and fish include, but are not limited to, lice, biting lice, ticks, nasal bots, keds, biting flies, muscoid flies, flies, myiasitic fly larvae, chiggers, gnats, mosquitoes and fleas.

The compounds of formula I and compositions comprising them are suitable for systemic and/or non-systemic control of ecto- and/or endoparasites. They are active against all or some stages of development.

Administration can be carried out both prophylactically and therapeutically. Administration of the active compounds is carried out directly or in the form of suitable preparations, orally, topically/dermally or parenterally.

For oral administration to warm-blooded animals, the formula I compounds may be formulated as animal feeds, animal feed premixes, animal feed concentrates, pills, solutions, pastes, suspensions, drenches, gels, tablets, boluses and capsules. In addition, the formula I compounds may be administered to the animals in their drinking water.

For oral administration, the dosage form chosen should provide the animal with 0.01 mg/kg to 100 mg/kg of animal body weight per day of the formula I compound, preferably with 0.5 mg/kg to 100 mg/kg of animal body weight per day. Alternatively, the formula I compounds may be administered to animals parenterally, for example, by intraruminal, intramuscular, intravenous or subcutaneous injection. The formula I compounds may be dispersed or dissolved in a physiologically acceptable carrier for subcutaneous injection. Alternatively, the formula I compounds may be formulated into an implant for subcutaneous administration. In addition the formula I compound may be transdermally administered to animals. For parenteral administration, the dosage form chosen should provide the animal with 0.01 mg/kg to 100 mg/kg of animal body weight per day of the formula I compound.

The formula I compounds may also be applied topically to the animals in the form of dips, dusts, powders, collars, medallions, sprays, shampoos, spot-on and pour-on formulations and in ointments or oil-in-water or water-in-oil emulsions. For topical application, dips and sprays usually contain 0.5 ppm to 5,000 ppm and preferably 1 ppm to 3,000 ppm of the formula I compound. In addition, the formula I compounds may be formulated as ear tags for animals, particularly quadrupeds such as cattle and sheep.

Suitable preparations are:
Solutions such as oral solutions, concentrates for oral administration after dilution, solutions for use on the skin or in body cavities, pouring-on formulations, gels;
Emulsions and suspensions for oral or dermal administration; semi-solid preparations;
Formulations in which the active compound is processed in an ointment base or in an oil-in-water or water-in-oil emulsion base;
Solid preparations such as powders, premixes or concentrates, granules, pellets, tablets, boluses, capsules; aerosols and inhalants, and active compound-containing shaped articles.

Generally it is favorable to apply solid formulations which release compounds of formula I in total amounts of 10 mg/kg to 300 mg/kg, preferably 20 mg/kg to 200 mg/kg. The active compounds can also be used as a mixture with synergists or with other active compounds which act against pathogenic endo- and ectoparasites.

In general, the compounds of formula I are applied in parasiticidally effective amount-meaning the amount of active ingredient needed to achieve an observable effect on growth, including the effects of necrosis, death, retardation, prevention, and removal, destruction, or otherwise diminishing the occurrence and activity of the target organism. The parasiticidally effective amount can vary for the various compounds/compositions used in the invention. A parasiticidally effective amount of the compositions will also vary according to the prevailing conditions such as desired parasiticidal effect and duration, target species, mode of application, and the like.

### SYNTHESIS EXAMPLES

With due modification of the starting compounds, the protocols shown in the synthesis example below were used or obtaining further compounds I. The resulting compounds, together with physical data, are listed in the table 1 which follows.

The products were characterized by coupled High Performance Liquid Chromatography/mass spectrometry (HPLC/MS), by ¹H-NMR (400 MHz) in CDCl₃ or d⁶-DMSO or by their melting points. HPLC column: RP-18 column (Chromolith Speed ROD from Merck KgaA, Germany). Elution: acetonitrile+0.1% trifluoroacetic acid (TFA)/water in a ratio of from 5:95 to 95:5 in 5 minutes at 40° C. MS: Quadrupol electrospray ionisation, 80 V (positiv modus).

### Example 1

### 2′,5′-dichloro-biphenyl-4-sulfonicacid (quinoline-4-ylmethyl)-amide (1-8)

### Step 1: 4-bromo-N-quinoline-4-yl-methyl-benzenesulfonamide

To 9.29 g (58.7 mmol) of quinoline-4-yl-methylamine and 8.91 g (88.05 mmol) triethylamine in 100 ml methylenechloride, 17.99 g (70 mmol) of 4-bromophenylsulfonylchloride were added and the reaction mixture was stirred at 20-25° C. for 16 hours. Water was added, the precipitate collected and washed three times with water, which gave 15.9 g of the title compound. ¹H-NMR (400 MHz (d⁶-DMSO): δ[ppm]=8.8 (m, 1H); 8.5 (t, 1H); 8.2-8.0 (m, 2H); 7.8-7.7 (m, 4H); 7.6 (m, 1H); 4.6 (d, 2H).

### Step 2: 2′,5′-dichloro-biphenyl-4-sulfonicacid (quinoline-4-yl-methyl)-amide (I-8)

302 mg (0.8 mmol) of 4-bromo-N-quinoline-4-yl-methyl-benzenesulfonamide (step 1), 305 mg (1.6 mmol) of 2,5-dichlorophenylboronic acid and 0.8 g (mmol) of polymer bound tetrakis-triphenylphosphin-palladium (0) in 10 ml of tetrahydrofuran were heated to reflux for 2 days, 300 mg of N,N-diethylaminomethyl polystyrene (Nova Biochem, 1.8 nmol/g) were added, and the mixture was agitated for additional 4 hours. Filtration, washing with tetrahydrofuran and column chromatography (with cyclohexane/ethyllacetate 7:3 eluent) yields the final product 1-8 228 mg as a solid, mp. 124° C.

### Example 2

### Preparation of 2′,4′-bis-trifluoromethyl-biphenyl-4-sulfonic acid (8-chloroquinoline-4-yl-methyl)-amide (I-22)

### Step 1: Preparation of 8-chloro-quinoline-4-carbonitrile

Pd(OAc)₂ (0.28 g, 1.26 mmol), Na₂CO₃ (2.94 g, 27.77 mmol), K₄[Fe(CN)₆]*3H₂O (4.69 g, 11.11 mmol) and 4,8-dichloro-quinoline (5.00 g, 25.25 mmol) were suspended in N,N-dimethylacetamide (60 ml) and heated to 120° C. for 3 days. Pd(OAc)₂ (0.28 g, 1.26 mmol) and K₄[Fe(CN)₆]*3H₂O (2.35 g, 5.56 mmol) were added and the reaction mixture was stirred for further 8 hours. After cooling to 20° C., the mixture was diluted with ethyl acetate, filtered and the solvents were evaporated. Column chromatographic purification (SiO₂, petrolether/ethylacetate 95:5→90:10→80:20) yielded 8-chloroquinoline-4-carbonitrile (3.50 g), [M+H]⁺: 169,00; retention time: 2.098 min.

### Step 2: preparation of 4-bromo-N-(8-chloro-quinoline-4-ylmethyl)benzene-sulfonamide

8-Chloro-quinoline-4-carbonitrile (1.50 g, 7.95 mmol) was dissolved in tetrahydrofuran (10 ml) and heated to reflux. BH_{3*}dimethylsulfid-complex (0.88 ml, 8.75 mmol) was added dropwise and the reaction mixture was stirred for 1.5 hours. After cooling to about 20° C., the solvent was evaporated and the residue taken up in tetrahydrofuran (10 ml). Aq. HCl (20%, 5.22 g, 28.63 mmol) was added, and the mixture was heated to reflux for 1.5 hours. The solvent was removed again and the residue taken up in Et₂O (10 ml). NEt₃ (3.9 ml, 27.83 mmol) was added and the mixture was stirred for 10 min. Then a solution of 4-bromobenzene sulfonyl chloride (2.85 g, 11.13 mmol) in Et₂O (10 ml) was added dropwise at 0° C. and the reaction mixture was stirred for 12 hours. The mixture was decanted and the residue taken up in ethyl acetate and water. The organic phase was separated and dried over Na₂SO₄. Column chromatographic purification (SiO₂, cyclohexane/ethylacetate 100:0→0:100) yielded 4-bromo-N-(8-chloroquinolin-4-ylmethyl)benzenesulfonamide, mp: 185° C.

### Step 3: preparation of 2′,4′-bis-trifluoromethyl-biphenyl-4-sulfonic acid (8-chloroquinoline-4-yl-methyl)-amide (I-22)

Analog to the preparation procedure described above for step 2 of example 1, the reaction of 0.25 g 4-bromo-N-(8-chloro-quinoline-4-ylmethyl)benzene-sulfonamide and 0.175 g bistrifluoromethylboronic acid yielded 0.15 g of the title compound (I-22), mp. 184-185° C.

### Example 3

### Preparation of Intermediates of the Formulae VIa and Va

Step 1: 5.00 g of 4,6,8-trimethylquinoline (VIe-1) (X═CH₃, R⁵, R⁷═CH₃, R⁴, R⁵═H) were added to a solution of 6.55 g (0.06 mol) of potassium-tert.-butylat in 100 ml of tetrahydrofuran at 0° C. and stirred at this temperature for 1.5 hours. Then 9.00 g (0.08 mol) tert.-butylnitrit were added drop wise and the mixture was stirred for 16 hours. Water and methyl-tertbutylether were added, the organic layer separated and the aqueous layer extracted again with Methyltertbutylether.

The combined organic extracts were washed with brine, dried over Na₂SO₄ and the solvent was evaporated, giving 4.4 g crude product VIa-1. ¹H-NMR (d⁶-DMSO): δ[ppm]=11.8 (s, 1H), 8.8 (m, 1H), 8.2 (s, 1H), 7.8 (m, 1H), 7.7 (m, 1H), 7.4 (m, 1H).

Step 2: 1.0 g (0.005 mol) crude product VIa-1 from the step above was hydrogenated at 1.1 bar in the presence of 4 g Raney Nickel in 200 ml methanol to yield after filtration and evaporation 900 mg of the title compound V-1. HPLC-MS: MS m/e [M+H⁺]=187.1, retention time: 1.16 min.

### Example 4

### Preparation of 8-fluoro-4-methylenaminoquinoline

### Step 1: 4-cyano-8-fluoro-quinoline (VIb-2)

1.0 g (0.0055 mol) 4-chloro-8-fluoroquinoline, 0.68 g Na₂CO₃, 0.58 g K₄[Fe(CN)₆], 0.14 g Pd-dibenzylidenacetone complex, and 0.183 g bisdiphenylphospino-ferrocen were heated to 140° C. in 10 ml N-methylpyrrolidon for 24 hours, cooled and diluted with 20 ml methylenehloride. Filtration and chromatography with ethylacetate/cyclohexane=7/3 yielded 0.998 g of 4-cyano-8-fluoro-quinoline VIb-2. HPLC-MS: m/e [M+H⁺]=173.0.

### Step 2: 8-fluoro 4-methylenaminoquinoline (V-2)

0.5 g (0.0029 mol) of compound VIb-2 from the step above in 100 ml methanol was hydrogenated in the presence of 0.06 g 10% Pd on charcoal and 1.5 ml of saturated aqueous ammonia for 20 hours, yielding 85 mg of the amine V-2.

### Example 5

### Preparation of 4-bromo-N-(6,8-dimethyl-quinoline-4-ylmethyl)-benzenesulfonamide (II-2)

From 0.9 g (0.005 mol) dimethyl-quinolineamine V-1, 1.23 (0.005 mol) 4-bromo-sulphonylchloride and 0.583 g (0.0058 mol) NEt₃, 0.8 g of the title compound was obtained as a colorless precipitate. ¹H-NMR: δ[ppm]=9.6 (d, 1H), 8.5 (m, 1H), 7.8-7.6 (m, 5H), 7.4-7.3 (m, 2H), 4.5 (d, 2H) 2.7 (s, 3H), 2.4 (s, 3H).

### Example 6

### Preparation of 4-bromo-N-(7-fluoro-quinoline-4-ylmethyl)-benzenesulfonamide (II-3)

Starting from 1.23 g (0.007 mol) 7-fluoro-quinolineamine, 1.78 g 4.bromo-sulphonylchloride and 0.85 g (0.008 mol) NEt₃ the title compound was obtained. ¹H-NMR: δ[ppm]=8.8 (d, 1H), 8.6 (m, 1H), 8.2 (m, 1H), 7.7-7.6 (m, 5H), 7.6 (m, 1H), 7.4 (m, 1H), 4.5 (d, 2H).

Following analogue routes, 4-bromo-N-(7-chloro-quinoline-4-ylmethyl)-benzenesulfonamide (II-4), HPLC-MS 412.9 2.65 min and 4-bromo-N-(8-fluoro-quinolin-4-ylmethyl)-benzenesulfonamide (II-5), HPLC-MS: MS m/e [M+H⁺]=396.9, retention time=2.67 min, were obtained.

### Example 7

### Preparation of 2′,4′-Bis-trifluoromethyl-biphenyl-4-sulfonic acid (1-oxy-quinoline-4-ylmethyl)-amide (N-oxide of I-10 (R¹, R²=2,4-(CF₃)₂, R⁴ to R⁷ are H)

0.50 g (0.001 mol) of compound I-10 in 50 ml acetonitrile were treated with 0.276 g (0.0012 mol) meta-chloroperbenzoic acid and stirred overnight at 20-25° C. The precipitate was filtered off, washed with methyl-tert-butylether and the volatiles removed in vacuo. The crude product was dissolved in 50 ml of methylenehloride and washed twice with saturated NaHCO₃ solution, brine and dried over MgSO₄ to yield 157 mg of the title compound. HPLC-MS: MS mie [M+H⁺]=527.1, retention time=3.39 min.

### II. Assessment of the Activity Against Animal Pest

1. Activity Against Boll Weevil (Anthonomus grandis)

The active compounds were formulated in 1:3 DMSO:water. 10 to 15 eggs were placed into microtiterplates filled with 2% agar-agar in water and 300 ppm formaline. The eggs were sprayed with 20 μl of the test solution, the plates were sealed with pierced foils and kept at 24-26° C. and 75-85% humidity with a day/night cycle for 3 to 5 days. Mortality was assessed on the basis of the remaining unhatched eggs or larvae on the agar surface and/or quantity and depth of the digging channels caused by the hatched larvae. Tests were replicated 2 times.

In this test, compounds I-1, I-2, I-3, I-4, I-5, I-6, I-7, I-8, I-9, I-10, I-11, I-12, I-13, I-14, I-15, I-16, I-17, I-19, I-20, I-27, I-28, I-29, I-30, I-33, I-37, I-38, I-39, I-40, I-41, I-42, I-43, I-46, and II-5 at 2500 ppm showed at least 75% mortality.

### 2. Activity Against Mediterranean Fruitfly (Ceratitis Capifata)

The active compounds were formulated in 1:3 DMSO:water. 50 to 80 eggs were placed into microtiterplates filled with 0.5% agar-agar and 14% diet in water. The eggs were sprayed with 5 μl of the test solution, the plates were sealed with pierced foils and kept at 27-29° C. and 75-85% humidity under fluorescent light for 6 days. Mortality was assessed on the basis of the agility of the hatched larvae. Tests were replicated 2 times.

In this test, compounds I-1, I-8, I-10, I-13, I.39, and I-42 at 2500 ppm showed at least 75% mortality.

### 3. Activity Against Tobacco Budworm (Heliothis Virescens)

The active compounds were formulated in 1:3 DMSO:water. 15 to 25 eggs were placed into microtiterplates filled with diet. The eggs were sprayed with 10 μl of the test solution, the plates were sealed with pierced foils and kept at 27-29° C. and 75-85% humidity under fluorescent light for 6 days. Mortality was assessed on the basis of the agility and of comparative feeding of the hatched larvae. Tests were replicated 2 times.

In this test, compounds I-1, I-4, I-6, I-8, I-10, I-13, I-14, I-15, I-17, I-19, I-20, I-35, I-36, and I-50 at 2500 ppm showed at least 75% mortality.

### 4. Activity Against Vetch Aphid (Megoura Viciae)

The active compounds were formulated in 1:3 DMSO:water. Bean leaf disks were placed into microtiterplates filled with 0.8% agar-agar and 2.5 ppm OPUS™. The leaf disks were sprayed with 2.5 μl of the test solution and 5 to 8 adult aphids were placed into the microtiterplates which were then closed and kept at 22-24° C. and 35-45% under fluorescent light for 6 days. Mortality was assessed on the basis of vital, reproduced aphids. Tests were replicated 2 times.

In this test, compound I-1, I-8, I-10, I-14, I-15, I-32, I-46, and I-50 at 2500 ppm showed at least 75% mortality compared to 0% mortality of untreated controls.

### 5. Cotton Aphid (Aphis Gossypil), Mixed Life Stages

The active compounds were formulated in 50:50 acetone:water and 100 ppm Kinetic® surfactant.

Cotton plants at the cotyledon stage were infested prior to treatment by placing a heavily infested leaf from the main aphid colony on top of each cotyledon. The aphids were allowed to transfer overnight and the host leaf was removed. The infested cotyledons were then dipped and agitated in the test solution for 3 seconds and allowed to dry in a fume hood. Test plants were maintained under fluorescent lighting in a 24-hr photoperiod at 25° C. and 20-40% relative humidity. Aphid mortality on the treated plants, relative to mortality on untreated check plants, was determined after 5 days.

In this test, the compounds I-1, I-3, I-4, I-5, I-6, I-7, I-8, I-9, I-10, I-11, I-12, I-13, I-14, I-15, I-16, I-19, I-20, I-21, I-25, I-27, I-28, I-30, I-31, I-32, I-34, I-35, I-36, I-38, I-39, I-45, I-49, and I-50 at 300 ppm showed a mortality of at least 80% in comparison with untreated controls.

6. Southern Armyworm (Spodoptera Eridanid), 2^{nd}-3^{rd} Instar Larvae

The active compounds were formulated as a 10.000 ppm solution in a mixture of 35% acetone and water, which was diluted with water, if needed.

Sieva lima bean foliage, expanded to the first true leaves, were dipped and agitated in the test solution for 3 seconds and then allowed to dry in a fume hood. The treated plant was then placed in 25-cm plastic perforated zip enclosure bags, ten 2^{nd}-instar larvae were added, and the bags sealed. After 4 days, observations were made of mortality, plant feeding, and of any interference with larval growth. In this test, the compounds I-1, I-2, I-4, I-6, I-8, I-9, I-10, I-11, I-12, I-13, I-14, I-15, I-16, I-17, I-18, I-20, I-21, I-22, I-25, I-28, I-31, I-35, I-36, I-38, I-39, I-40, I-42, and I-52 at 300 ppm showed a mortality of at least 80% in comparison with untreated controls.

### 7. Tobacco Budworm (Heliothis Virescens)

Two-leaf cotton plants were utilized for bioassays. Excised plant leaves were dipped into 1:1 acetone/water dilutions of the active compounds. After the leaves had dried, they were individually placed onto water-moistened filter paper on the bottoms of Petri dishes. Each dish was infested with 5-7 larvae and covered with a lid. Each treatment dilution was replicated 4 times. Test dishes were held at approximately 27° C. and 60% humidity. Numbers of live and morbid larvae were assessed in each dish at 5 days after treatment application, and percent mortality was calculated.

In this test, the compounds I-35, I-36 and I-50 at 300 ppm showed a mortality of at least 80% in comparison with untreated controls.

### 8. Colorado Potato Beetle (Leptinotarsa Decemlineata)

Potato plants were utilized for bioassays. Excised plant leaves were dipped into 1:1 acetone/water dilutions of the active compounds. After the leaves had dried, they were individually placed onto water-moistened filter paper on the bottoms of Petri dishes. Each dish was infested with 5-7 larvae and covered with a lid. Each treatment dilution was replicated 4 times. Test dishes were held at approximately 27° C. and 60% humidity. Numbers of live and morbid larvae were assessed in each dish at 5 days after treatment application, and percent mortality was calculated.

In this test, the compounds I-14, I-17, I-18, I-19, I-20, I-21, I-22, I-24, I-25, I-34, I-41, I-42, I-44, I-45, and I-52 at 300 ppm showed a mortality of at least 75% in comparison with untreated controls.

### 9. Green Peach Aphid (Myzus Persicae)

The active compounds were formulated in 50:50 acetone:water and 100 ppm Kinetic™ surfactant.

Pepper plants in the 2^{nd} leaf-pair stage (variety California Wonder) were infested with approximately 40 laboratory-reared aphids by placing infested leaf sections on top of the test plants. The leaf sections were removed after 24 hr. The leaves of the intact plants were dipped into gradient solutions of the test compound and allowed to dry. Test plants were maintained under fluorescent light (24 hour photoperiod) at about 25° C. and 20-40% relative humidity. Aphid mortality on the treated plants, relative to mortality on check plants, was determined after 5 days.

In this test, the compounds I-1, I-4, I-6, I-8, I-9, I-10, I-11, I-12, I-14, I-15, I-19, I-20, I-21, I-26 and I-31 at 300 ppm showed a mortality of at least 75% in comparison with untreated controls.

10. Silverleaf whitefly (Bemisia Argentifolii)

The active compounds were formulated in 50:50 acetone:water and 100 ppm Kinetic™ surfactant.

Selected cotton plants were grown to the cotyledon state (one plant per pot). The cotyledons were dipped into the test solution to provide complete coverage of the foliage and placed in a well-vented area to dry. Each pot with treated seedling was placed in a plastic cup and 10 to 12 whitefly adults (approximately 3-5 day old) were introduced. The insects were colleted using an aspirator and an 0.6 cm, non-toxic Tygon tubing connected to a barrier pipette tip. The tip, containing the collected insects, was then gently inserted into the soil containing the treated plant, allowing insects to crawl out of the tip to reach the foliage for feeding. The cups were covered with a re-usable screened lid (150 micron mesh polyester screen PeCap from Tetko Inc). Test plants were maintained in the holding room at about 25° C. and 20-40% relative humidity for 3 days avoiding direct exposure to the fluorescent light (24 hour photoperiod) to prevent trapping of heat inside the cup. Mortality was assessed 3 days after treatment of the plants.

In this test, the compounds I-1, I-5, I-7, I-8, I-10, I-11, I-12, I-19, I-20, I-1, I-10, and I-45 at 300 ppm showed a mortality of at least 70% in comparison with untreated controls.

### 11. 2-Spotted Spider Mite (Tetranychus Urticae, OP-Resistant Strain)

The active compounds were formulated in 50:50 acetone:water and 100 ppm Kinetic™ surfactant.

Sieva lima bean plants with primary leaves expanded to 7-12 cm were infested by placing on each a small piece from an infested leaf (with about 100 mites) taken from the main colony. This was done at about 2 hours before treatment to allow the mites to move over to the test plant to lay eggs. The piece of leaf used to transfer the mites was removed. The newly-infested plants were dipped in the test solution and allowed to dry. The test plants were kept under fluorescent light (24 hour photoperiod) at about 25° C. and 20-40% relative humidity. After 5 days, one leaf was removed and mortality counts were made.

In this test, the compound I-46 at 300 ppm showed a mortality of at least 70% in comparison with untreated controls.

### 12. Activity Against Cowpea Aphid (Aphis Craccivora)

The active compounds were formulated in 50:50 acetone:water. Potted cowpea plants colonized with 100-150 aphids of various stages were sprayed after the pest population has been recorded. Population reduction was recorded after 24, 72, and 120 hours.

In this test, the compounds I-1, I-4, I-5, I-6, I-8, I-9, I-10, I-11, I-12, I-13, I-14, I-15, I-18, I-19, I-20, I-21, I-24, I-24, I-37, I-39, I-42 and I-43 at 300 ppm showed a mortality of at least 80% in comparison with untreated controls.

13. Activity Against Diamond Back Moth (plutella Xylostella)
The active compounds were formulated in 50:50 acetone:water and 0.1% (vol/vol) Alkamuls EL 620 surfactant. A 6 cm leaf disk of cabbage leaves was dipped in the test solution for 3 seconds and allowed to air dry in a Petri plate lined with moist filter paper. The leaf disk was inoculated with 10 third instar larvae and kept at 25-27° C. and 50-60% humidity for 3 days. Mortality was assessed after 72 h of treatment.

In this test, the compounds I-1, I-2, I-4, I-6, I-8, I-10, I-11, I-15 I-19, I-26, I-35, I-36 and I-50 at 300 ppm showed a mortality of at least 75% in comparison with untreated controls.

### 14. Activity Against Yellowfever Mosquitos (Aedes Aegypti) Via Water Treatment

The test compound (1 Vol % in acetone) was applied to 9 ml distilled water In polystyrene plastic 6-well plates. Treatment rates were applied 1 and 10 ppm. Ten, 4^{th} instar yellowfever mosquito larvae, Aedes aegypti, were added to each well in 1 ml of distilled water. The test dishes were maintained at 22° C. and observed daily for mortality, up to 5 days after treatment. Each treatment was replicated in 6 wells.

In this assay, compound I-10 provided 52% mortality at 10 ppm after 5 days. Compound I-1 provided 100% mortality at 1 ppm after 3 days.

15. Activity Against Eastern Subterranean Termites (Reticulitérmes Flávipes) Via Termite Filter Paper Bait.

Toxicant treatments (0.1 and 0.3% test compound w/w) were applied to 4.25 cm (diam.) filter papers in acetone solution. Treatment levels (% test compound) were calculated on basis of a mean weight per filter paper of 106.5 mg. Treatment solutions were adjusted to provide the quantity of toxicant (mg) required per paper in 213 ml of acetone. Acetone only was applied for untreated controls. Treated papers were vented to evaporate the acetone, moistened with 0.25 ml water, and placed in corresponding test arenas.

Termite bait bioassays were conducted in 100×15 mm Petri dishes with 1 teaspoon fine sand spread in a thin layer over the bottom of each dish. An additional 0.25 teaspoon sand was piled against the side of each dish. The sand was moistened with 2.7 ml water applied to the piled sand. Water was added to dishes as needed over the course of the bioassays to maintain high moisture content. Bioassays had one treated filter (on the sand) and 30 termite workers per test dish. Each treatment level was replicated in 3 test dishes. Test dishes were maintained at approximately 30° C. and 85% humidity for 14 days and observed daily for mortality.

In this assay, compound I-10 provided 86% mortality at 0.3% w/w after 14 days. Compound I-1 provided 99% mortality at 0.1% w/w after 7 days.

16. Activity Against Acrobat Ant (Crematogaster sp.), House Fly (Musca domestica), Yellowfever Mosquito (Aedes aegypti) , Via Glass Contact.

Glass vials (20 ml scintillation vials) were treated with 0.44 ml of a solution of active ingredient in acetone. Each vial was rolled uncapped for ca. 10 minutes to allow the active ingredient to completely coat the vial and to allow for full drying of the acetone. Six insects or ticks were placed into each vial, with 5 vials per treatment rate. The vials are kept at 22° C. and are observed for treatment effects at various time intervals.

In the yellowfever mosquito glass vial assay, compound I-10 showed 100% mortality at 10 ppm after 24 hours, and compound I-1 provided 37% mortality at 10 ppm after 48 hours.

In the housefly mosquito glass vial assay, compound I-10 showed 80% mortality at 10 ppm after 24 hours, and compound I-1 provided 30% mortality at 10 ppm after 48 hours.

In the acrobat ant glass vial assay, compound I-10 showed 86% mortality at 100 ppm after 2 days and compound I-1 provided 7% mortality at 100 ppm after 4 days.

17. Activity Against Acrobat Ant (Crematogaster sp.) and Subterranean Termites (Reticulitermes flavipes) Via Soil Incorporation.

Soil was prepared by adding stock solutions (active dissolved in acetone) to 100 g sandy loam soil to make 1, 10, and 100 ppm batches. Jars of soils were rolled on a jar roller to mix thoroughly and then allowed to vent over night with the acetone evaporating off.

Ant bioassays were conducted in 100×20 mm Petri dishes and were prepared with a layer of 1% agar (7 ml) and then 12.75 g of dry treated soil. A cotton pellet (#2) soaked with a 10% sugar solution was added to each dish in a micro-weigh boat as a source of moisture and food. Each dish was then infested with 15 acrobat ant workers, Crematogaster sp. Three replicates were setup per treatment rate. Mortality was observed daily up to 7 days with moribund and dead ants removed at each reading.

Subterranean termite bioassays were conducted in 50×15 mm Petri dishes and were prepared with a layer of 1% agar (2 ml) and then 2 g of dry treated soil. A 1 cm² piece of filter paper, moistened with 1 drop of distilled water was added to each dish as a source of cellulose food. Each dish was then infested with 15 eastern subterranean termite workers, Reticulitermes flavipes. Five replicates were setup per treatment rate. Mortality was observed daily up to 7 days with moribund and dead termites removed at each reading.

In the acrobat ant soil incorporation assay, compound I-10 showed 100% mortality at 100 ppm after 3 days and compound I-1 provided 36% mortality at 100 ppm after 7 days. In the termite soil incorporation assay, compound I-10 showed 84% mortality at 100 ppm after 9 days and compound I-1 provided 95% mortality at 100 ppm after 9 days.

18. Orchid Thrips (dichromothrips corbetti)

Dichromothrips corbefti adults used for bioassay are obtained from a colony maintained continuously under laboratory conditions. For testing purposes, the test compound is diluted to a concentration of 500 ppm (wt compound: vol diluent) in a 1:1 mixture of acetone:water, plus 0.01% Kinetic surfactant.

Thrips potency of each compound is evaluated by using a floral-immersion technique. Plastic petri dishes are used as test arenas. All petals of individual, intact orchid flowers are dipped into treatment solution for approximately 3 seconds and allowed to dry for 2 hours. Treated flowers are placed into individual petri dishes along with 10-15 adult thrips. The petri dishes are then covered with lids. All test arenas are held under continuous light and a temperature of about 28° C. for duration of the assay. After 4 days, the numbers of live thrips are counted on each flower, and along inner walls of each petri dish. The level of thrips mortality is extrapolated from pre-treatment thrips numbers.

### 19. Activity Against Flea Beetle (Phylotrefta Striolata)

Flea bettle (Phylotrefta striolata) adults used for bioassay were obtained from a colony maintained continuously under laboratory conditions. For testing purposes, the test compound was diluted to a concentration of 300 ppm (wt compound: vol diluent) in a 1:1 mixture of acetone:water, plus 0.1% EL 620 surfactant.

Activity of each compound was evaluated using a lip-dip technique. Glass petri dishes (60×15 mm) lined with moist filter paper serves as test arenas. All leaf discs were dipped into treatment solution for approximately 3 seconds and allowed to dry for 2 hours. Each treated leaf disc was placed into individual petri dishes and inoculated with 10 adult beetles. Petri dishes were then covered with lids. All test arenas were held under continuous light and a temperature of about 28° C. for duration of the assay. After 3 days, percent mortality is observed.

At 300 ppm the following compounds showed a mortality of 75% or higher: I-32, I-34, I-37, I-42 and I-50.

20. Activity Against Rice Green Leaf Hoppers (Nephoteftix Virescens) and Brown Plant Hoppers (Nilaparvata lugens).

Leaf hopper and plant hoppers adults used for bioassay were obtained from a colony maintained continuously under laboratory conditions. For testing purposes, the test compound was diluted in 300 ppm (wt compound:vol diluent) in a 1:1 mixture of acetone:water, plus 0.1% EL 620 surfactant.

Hopper activity was evaluated using the foliar spray technique. Pofted rice plants (2-3 week old, Variety TN-1) were cleaned and dried prior to application. All plants were treated inside the fume hood using DeVilbiss atomizer at 25 psi with a spray volume of 5 ml/plant. To ensure uniform spray distribution, plants were placed in a rotating flat form inside the fume hood. Treated plants were then placed inside the holding room and allowed to dry for 2 hours. Each plant was caged using Mylar cages (4 inches diameter×19 inches in height) and inoculated with 10 adults hoppers. All test plants were held under continuous light and a temperature of about 28° C. for duration of the assay. Percent mortality was observed after 72 hours.

At 300 ppm the following compounds showed 75% mortality or higher against the green leaf hopper: I-32.

TABLE I

| | |
|---|---|
| | (I) |
| 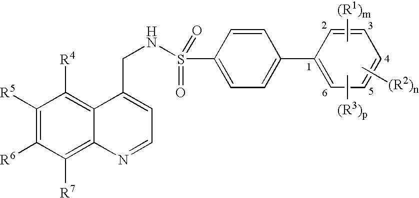 | |

| | | | | | | | | phys. data (mp. [° C.]; ¹H-NMR δ[ppm]; |
|---|---|---|---|---|---|---|---|---|
| No. | (R¹)ₘ | (R²)ₙ | (R³)ₚ | R⁴ | R⁵ | R⁶ | R⁷ | MS m/e [M + H⁺], retention time [min]) |
| I-1 | 2-Cl | 4-Cl | — | H | H | H | H | 181-182; δ(CDCl₃): 8.8 (m); 8.1 (m); 7.8 (m); 7.7 (m); |
| | | | | | | | | 7.7-7.5 (m); 7.4-7.2 (m); 5.3 (t); 4.7(d). [M + H⁺]: 443.4; rt = |
| | | | | | | | | 3.07 min |
| I-2 | 3-CH₃ | 5-CH₃ | — | H | H | H | H | 193-194; δ(CDCl₃): 8.8 (m); 8.2 (m); 7.8 (m); 7.7-7.6 (m); |
| | | | | | | | | 7.6 (m); 7.3 (m), 7.2 (m); 7.1 (ps); 5.7 (t); 4.6 (d); 2.4 (s). |
| I-3 | 3-OCH₃ | 4-OCH3 | — | H | H | H | H | 190-192; δ(d⁶DMSO): 8.8 (m); 8.4 (s); 8.2 (psd); 8.0 |
| | | | | | | | | (psd); 7.9-7.8 (m); 7.8 (m); 7.7 (m); 7.5 (m); 7.3 (m); 7.1 |
| | | | | | | | | (m); 4.6 (s); 3.8 (s); 3.7 (s) |
| I-4 | 3-F | 4-OCF₃ | — | H | H | H | H | 131-132; δ(d⁶DMSO): 8.8 (s); 8.5 (s); 8.2 (m); 8.0-7.8 |
| | | | | | | | | (m); 7.8-7.7 (m); 7.5 (m); 7.4 (m); 4.6 (s); |
| I-5 | 2-CH₃ | 3-CH₃ | — | H | H | H | H | 149; δ(d⁶DMSO): 8.8 (m); 8.5 (s); 8.2 (m); 8.0 (m); 7.8 |
| | | | | | | | | (m); 7.7 (m); 7.6 (m); 7.5 (m); 7.3-7.2 (m), 7.1 (m); 4.6 |
| | | | | | | | | (s); 2.3 (s); 2.1 (s). |
| I-6 | 2-Cl | 4-CF₃ | — | H | H | H | H | 160; δ(d⁶DMSO): 8.8 (m); 8.1-8.0 (m); 7.9 (m); 7.7-7.4 |
| | | | | | | | | (m), 7.4-7.3 (m); 4.6 (d). |
| I-7 | 2-Cl | 3-Cl | — | H | H | H | H | 185; δ(d⁸THF): 8.8 (m); 8.1-8.0 (m); 7.9 (m); 7.7 (m); 7.6- |
| | | | | | | | | 7.4 (m); 4.6 (d). |
| I-8 | 2-Cl | 5-Cl | — | H | H | H | H | 124; δ(d⁶DMSO): 8.8 (m); 8.6 (s); 8.3 (m); 8.1-7.6 (m); |
| | | | | | | | | 7.4 (m); 4.6 (s). |
| I-9 | 2-Cl | 4-OCH₃ | — | H | H | H | H | 190; δ(d⁶DMSO): 8.8 (m), 8.5 (s); 8.2 (m); 8.0 (m); 7.9 |
| | | | | | | | | (m); 7.7 (m); 7.6-7.5 (m); 7.4 (m), 7.3 (m), 7.2 (m), 7.0 |
| | | | | | | | | (m); 4.6 (s); 3.8 (s). |
| I-10 | 2-CF₃ | 4-CF₃ | — | H | H | H | H | 179; δ(d⁶DMSO): 8.8 (m); 8.6 (br); 8.3-8.1 (m), 8.0 (m); |
| | | | | | | | | 7.9 (m); 7.8-7.6 (m); 7.6 (m); 7.4 (m); 4.6 (s); [M + H⁺]: |
| | | | | | | | | 511,5; rt = 3.21 min |
| I-11 | 2-CF₃ | 4-Cl | — | H | H | H | H | 192; δ(d⁸THF): 8.7 (m); 8.1-8.0 (m); 7.9 (m); 7.8 (m), 7.7 |
| | | | | | | | | (m); 7.5-7.3 (m); 4.6 (m). |
| I-12 | 2-CH₃ | 4-OCH₃ | — | H | H | H | H | 190; δ(d⁸THF): 8.8 (m); 8.1-8.0 (m); 7.9 (m); 7.7 (m); 7.6- |
| | | | | | | | | 7.4 (m); 4.6 (d). |
| I-13 | 2-CF₃ | 5-F | — | H | H | H | H | δ(d⁶DMSO): 8.8 (m); 8.6 (br); 8.1 (m); 8.1-7.7 (m); 7.7 |
| | | | | | | | | (m); 7.6 (m); 7.6-7.3 (m); 4.6 (d). |
| I-14 | 3-CH₃ | 4-Cl | — | H | H | H | H | 162-163; δ(d⁸THF): 8.8 (m); 8.1-8.0 (m); 7.9 (m); 7.8 (m); |
| | | | | | | | | 7.7 (m); 7.5-7.4 (m); 7.4 (m); 7.2 (m); 4.6 (d). |
| I-15 | 2-CH₃ | 4-Cl | — | H | H | H | H | 134-136; δ(d⁸THF): 8.8 (m); 8.1-8.0 (m); 7.9 (m); 7.7 (m); |
| | | | | | | | | 7.5 (m); 7.4 (m); 7.4-7.3 (m); 7.3 (m); 7.2 (m). 4.6 (d), 2.2 |
| | | | | | | | | (s). |
| I-16 | 2-OCH₃ | 4-Cl | | H | H | H | H | 196-197; δ(d⁸THF): 8.7 (m); 8.2-8.0 (m); 7.8 (m); 7.7-7.6 |
| | | | | | | | | (m); 7.5 (m); 7.4-7.3 (m); 7.3 (t); 7.2 (s); 7.1 (m); 4.6 (d); |
| | | | | | | | | 3.8 (s). |
| I-17 | 3-CF₃ | 5-CF₃ | — | H | H | H | H | 183; δ(d⁶DMSO): 8.8 (m); 8.5 (br); 8.4 (pss); 8.2 (s); 8.1 |
| | | | | | | | | (m); 8.1-7.9 (m); 7.7 (m); 7.6 (m); 7.4 (m, 1H), 4.6 (d) |
| I-18 | 2-Cl | 5-CF₃ | — | H | H | H | H | δ(d⁸THF): 8.8 (m); 8.1-8.0 (m); 7.9 (m); 7.8-7.7 (m); 7.7 |
| | | | | | | | | (m); 7.6 (m); 7.5 (m); 7.4 (m); 4.6 (s). |
| I-19 | 3-F | 4-Cl | — | H | H | H | H | 158-159; δ(d⁸THF): 8.7 (d); 8.1-8.0 (m); 7.9 (m); 7.8 (m); |
| | | | | | | | | 7.7-7.5 (m); 7.3 (m); 4.6 (s). |
| I-20 | 3-CF₃ | 4-Cl | — | H | H | H | H | 194-195; δ(d⁸THF): 8.8 (m); 8.2 (brs); 8.1-7.5 (m); 7.4 |
| | | | | | | | | (m); 4.6 (s). |
| I-21 | 3-Cl | 4-Cl | — | H | H | H | H | 93-98; δ(d⁸THF): 8.8 (m); 8.1-8.0 (m); 7.9 (m); 7.8 (m); |
| | | | | | | | | 7.7-7.6 (m); 7.5 (m); 7.4 (m). 4.6 (d). |
| I-22 | 2-CF₃ | 4-CF₃ | — | H | H | H | Cl | 184-185; δ(d⁸THF): 8.8 (m), 8.2 (br), 8.1 (m), 7.9 (m); 7.8 |
| | | | | | | | | (m); 7.6 (m); 7.6-7.4 (m); 4.6 (d). |
| I-23 | 5-NO₂ | 4-Cl | | H | H | H | H | 453.9; 2.88 min |
| I-24 | 2-Cl | 4-Cl | — | H | H | H | Cl | 478.0 |
| I-25 | 2-F | 4-Br | — | H | H | H | H | 473.2; 3.00 min |
| I-26 | 2-F | 4-Cl | — | H | H | H | H | 427.3; 2.94 min |
| I-27 | 3-F | 4-OCH₂CH₃ | — | H | H | H | H | 437.1; 2.78 min |
| I-28 | 3-Cl | 4-OCH₃ | — | H | H | H | H | 439.0 ; 2.74 min |
| I-29 | 3-Cl | 4- | — | H | H | H | H | 467.1; 3.18 min |
| | | O(CH₂)₂CH₃ | | | | | | |
| I-30 | 2-Cl | 4-OCH₂CH₃ | — | H | H | H | H | 453.1; 2.98 min |
| I-31 | 2-F | 4-Cl | 5- | H | H | H | H | 457.0; 2.90 min |
| | | | OCH₃ | | | | | |
| I-32 | 2-Cl | 4-CH₃ | — | H | H | H | H | 423.1; 2.96 min |
| I-33 | 3-F | 4-OCH₃ | — | H | H | H | H | 423.1; 2.65 min |
| I-34 | 2-CH₃ | 4-CH₃ | — | H | H | H | H | 403.1; 2.94 min |
| I-35 | 2-CH₃ | 4-CH₃ | 5- | H | H | H | H | 417.2; 3.07 min |
| | | | CH₃ | | | | | |
| I-36 | 2-F | 5-CH₃ | — | H | H | H | H | 407.1; 2.81 min |
| I-37 | 3-F | 4-CH₃ | — | H | H | H | H | 407.1; 2.84 min |
| I-38 | 3-Cl | 4-CH₃ | — | H | H | H | H | 423.1; 3.02 min |
| I-39 | 2-F | 5-Cl | — | H | H | H | H | 427.0; 2.81 min |
| I-40 | 3-CH₃ | 4-OCH₃ | — | H | H | H | H | 429.5; 2.82 min |
| I-41 | 2-OCH₃ | 3-OCH₃ | — | H | H | H | H | 435.1; 2.52 min |
| I-42 | 2-F | 5-Br | — | H | H | H | H | 471.0; 2.87 min |
| I-43 | 2-OCH₃ | 4-OCH₃ | — | H | H | H | H | 435; 2.72 min |
| I-44 | 2-Cl | 4-Cl | — | H | H | Cl | H | 479.0; 3.56 min |
| I-45 | 2-Cl | 4-Cl | — | H | H | H | F | 461.0; 3.55 min |
| I-46 | 2-CH₃ | 4-CF(CF₃)₂ | — | H | H | H | H | 168-169; 556.9; 3.55 min |
| I-47 | 2-CF₃ | 4-CF₃ | — | H | CH₃ | H | CH₃ | 538.9/3.41 |
| I-48 | 2-Cl | 4-Cl | — | H | CH₃ | H | CH₃ | 470.1/3.31 |
| I-49 | 2-Cl | 4-Cl | — | H | H | F | H | 461.4/3.23 |
| I-50 | 2-CF₃ | 4-CF₃ | — | H | H | F | H | 528.9/3.38 |
| I-51 | 2-F | 4-CHO | — | H | H | H | H | 420.9/2.42 |
| I-52 | 2-F | 4-CN | — | H | H | H | H | 418.3/2.50 |
| I-53 | 2-F | 5-CN | — | H | H | H | H | 418.3/2.47 |

We claim:

1-16. (canceled)

17. A compound of formula I 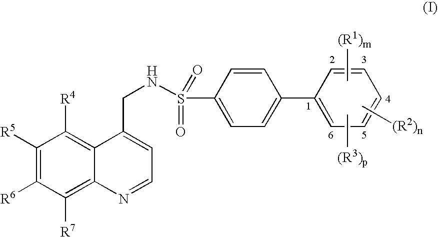
wherein
R¹, R² and R³ are each independently selected from the group consisting of halogen, hydroxy, cyano, amino, nitro, C₁-C₆-alkyl, C₂-C₆-alkenyl, C₂-C₆-alkynyl, C₃-C₇-cycloalkyl, C₃-C₇-cycloalkyl-C₁-C₄-alkyl, C₁-C₆-alkoxy, C₂-C₆-alkenyloxy, C₂-C₆-alkynyloxy, C₁-C₄-alkoxy-C₁-C₄-alkoxy, C₃-C₇-cycloalkyl-C₁-C₄-alkoxy, C(OH)(CF₃)₂, C₁-C₆-haloalkyl, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C(R^{a})═O, C(R^{a})═NOR^{b}, C(═O)OR^{x}, and C(═O)NR^{x}R^{y};
R^{a} is hydrogen or C₁-C₄-alkyl;
R^{b} is hydrogen, C₁-C₄-alkyl, C₂-C₄-alkenyl, C₂-C₄-alkynyl, C₁-C₄-haloalkyl, or
C₂-C₄-haloalkenyl;
R^{x} and R^{y} are each independently selected from the group consisting of hydrogen, C₁-C₄-alkyl, C₁-C₄-haloalkyl, C₁-C₄-alkoxy-C₁-C₄-alkyl, C₁-C₄-thioalkyl-C₁-C₄-alkyl, C₁-C₄-alkyl-S(═O)C₁-C₄-alkyl, C₁-C₄-alkyl-S(═O)₂C₁-C₄-alkyl, C₃-C₆-cyloalkyl, C₁-C₄-alkyl-C₃-C₆-cycloalkyl, C₃-C₆-alkenyl, and C₃-C₆-alkinyl;
R⁴, R⁵, R⁶ and R⁷ are each independently selected from the group consisting of hydrogen, halogen, cyano, amino, nitro, hydroxy, C₁-C₆-alkyl, C₁-C₆-alkoxy, C₁-C₆-haloalkyl, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, and C(═O)OR^{c};
R^{c} is hydrogen, C₁-C₆-alkyl, C₂-C₆-alkenyl, or C₂-C₆-alkinyl; provided that if R⁴ and R⁷ are hydrogen and R⁵ and R⁶ are selected from the group consisting of hydrogen, methyl, fluorine, chlorine, methoxy and trifluoromethoxy then R⁵ and R⁶ are different from each other;
m and n are each independently 1, 2, 3, 4, or 5;
p is 0, 1, 2, 3, 4, or 5;
and the N-oxides, enantiomers, diastereomers and salts thereof.

18. The compound of claim 17, wherein,
R¹, R² and R³ are each independently selected from the group consisting of halogen, cyano, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-haloalkyl, C₁-C₆-haloalkoxy, C₁-C₆-alkylthio, C₁-C₆-haloalkylthio, C₁-C₆-alkylsulfonyl, C₁-C₆-haloalkylsulfonyl, and C(R^{a})═NOR^{b};
m and n are each independently 1, 2, 3, 4, or 5; and
p is 0, 1, 2, 3, 4, or 5.

19. The compound of claim 17, wherein R⁴, R⁵, R⁶ and R⁷
are each independently selected from the group consisting of hydrogen, halogen, C₁-C₆-alkyl, C₁-C₆-alkoxy, C₁-C₆-haloalkyl, C₁-C₆-haloalkoxy, C₁-C₆-alkylthio, and C₁-C₆-haloalkylthio.

20. A process for the preparation of a compound of claim 17, comprising reacting sulfonylchlorides (IV) with quinolines (V) in the presence of a base to give intermediates (II), 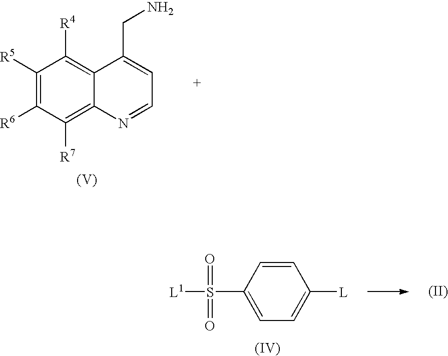
which intermediates (II) are subsequently reacted with boronic acids (III) by a Suzuki coupling in the presence of a base and a catalyst to give quinoline derivatives of formula I, 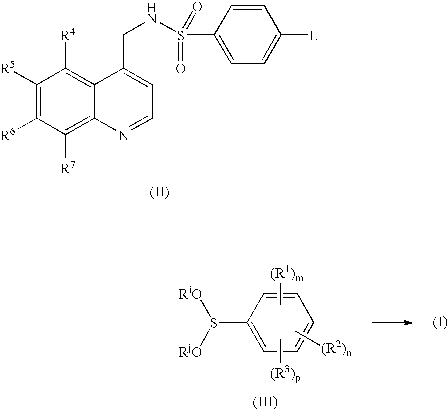
wherein, the variables in the above compounds have the meaning as defined above for quinoline derivatives of formula (I), Rⁱ and R^{j} are each independently hydrogen or C₁-C₄-alkyl, or Rⁱ and R^{j} together form an ethylene or propylene bridge the carbon atoms of which may all or in part be substituted by methyl groups, and L and L¹ are suitable leaving groups.

21. A compound of formula II.1 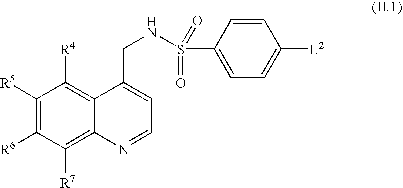
wherein, L² is chlorine, bromine or iodine, and
R⁴, R⁵, R⁶ and R⁷ are each independently selected from the group consisting of hydrogen, halogen, cyano, amino, nitro, hydroxy, C₁-C₆-alkyl, C₁-C₆-alkoxy, C₁-C₆-haloalkyl, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, and C(═O)OR^{c};
R^{c} is hydrogen, C₁-C₆-alkyl, C₂-C₆-alkenyl, or C₂-C₆-alkinyl;
provided that if R⁴ and R⁷ are hydrogen and R⁵ and R⁶ are selected from the group consisting of hydrogen, methyl, fluorine, chlorine, methoxy and trifluoromethoxy then R⁵ and R⁶ are different from each other.

22. A method for the control of insects, acarids or nematodes by contacting the insect, acarid or nematode or their food supply, habitat, breeding ground or their locus with a pesticidally effective amount of a compound(s) or a composition comprising said compound(s) of claim 17.

23. A method of protecting growing plants from attack or infestation by insects, acarids or nematodes by applying to the plants, or to the soil or water in which they are growing, a pesticidally effective amount of a compound(s) or composition comprising said compound(s) of claim 17.

24. The method of claim 22, wherein said compound(s) or said composition are applied in an amount of from 5 g/ha to 2000 g/ha.

25. The method of claim 23, wherein said compound(s) or said composition are applied in an amount of from 5 g/ha to 2000 g/ha.

26. A method of protection of seed comprising contacting the seeds with a compound(s) or a composition comprising compound(s) of claim 17 in pesticidally effective amounts.

27. The method of claim 26 wherein said compound(s) or said composition is applied in an amount of from 0.1 g to 10 kg per 100 kg of seeds.

28. A seed, comprising a compound(s) of claim 17 in an amount of from 0.1 g to 10 kg per 100 kg of seeds.

29. A method for treating, controlling, preventing or protecting animals against infestation or infection by parasites which comprises orally, topically or parenterally administering or applying to the animals a parasiticidally effective amount of a compound(s) of claim 17 or a composition comprising said compound(s) or their veterinarily acceptable salts.

30. Compositions comprising a pesticidally or parasiticidally active amount of a compound(s) of claim 17 and an agronomically or veterinarily acceptable carrier.

31. Synergistic pesticidal mixtures, comprising a compound of claim 17 and a pesticide selected from the organo(thio)phosphates, carbamates, pyrethroids, juvenile hormone mimics, nicotinic receptor agonists/antagonists compounds, GABA gated chloride channel antagonist compounds, chloride channel activators, METI I, II, and III compounds, uncouplers of oxidative phosphorylation, inhibitors of oxidative phosphorylation, moulting disruptors, synergists, sodium channel blocker compounds, fumigants, selective feeding blockers, mite growth inhibitors, chitin synthesis inhibitors, lipid biosynthesis inhibitors, octapaminergic agonists, ryanodine receptor modulators, aluminium phosphide, amidoflumet, benclothiaz, benzoximate, bifenazate, borax, bromopropylate, cyanide, cyenopyrafen, cyflumetofen, chinomethionate, dicofol, fluoroacetate, phosphine, pyridalyl, pyrifluquinazon, sulfur, tartar emetic; N—R′-2,2-dihalo-1-R″cyclo-propanecarboxamide-2-(2,6-dichloro-α,α,α,-tri-fluoro-p-tolyl)hydrazone or N—R′-2,2-di(R′″)propionamide-2-(2,6-dichloro-α,α,α,-trifluoro-p-tolyl)-hydrazone, wherein R′ is methyl or ethyl, halo is chloro or bromo, R″ is hydrogen or methyl and R′″ is methyl or ethyl, chloranthraniliprole, the compound of formula Γ² 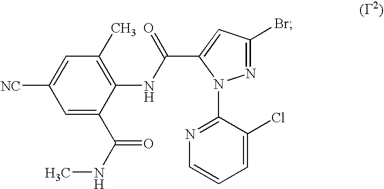
CF₃(CH₂)₂C(CN)₂CH₂(CF₂)₃CF₂H, CF₃(CH₂)₂C(CN)₂CH₂(CF₂)₅CF₂H, CF₃(CH₂)₂C(CN)₂(CH₂)₂C(CF₃)₂F, CF₃(CH₂)₂C(CN)₂(CH₂)₂(CF₂)₃CF₃, CF₃(CF₂)₂CH₂C(CN)₂CH₂(CF₂)₃CF₂H, CF₃CF₂CH₂C(CN)₂CH₂(CF₂)₃CF₂H, 2-(2,2,3,3,4,4,5,5-octafluoropentyl)-2-(3,3,4,4,4-pentafluorobutyl)-malonodinitrile, and CF₂HCF₂CF₂CF₂CH₂C(CN)₂CH₂CH₂CF₂CF₃
wherein said compound of claim 17 and said pesticide are present in a synergistic amount.
